SEQUENCE LISTING (13 sequences)

SEQ ID NO: 1 - DNA, 19 bp, rat
cgagcaggtgtatcaggaa

SEQ ID NO: 2 - DNA, 19 bp, rat
caaaggcattgagtactta

SEQ ID NO: 3 - DNA, 17 bp, rat
ccggatgttggacaaga

SEQ ID NO: 4 - protein, 23 aa, rat
RKPVDEYKDCHLAQVPSHTVVAR

SEQ ID NO: 5 - protein, 30 aa, rat
YLGYEYVTAIRNLREGTCPEAPTDECKPVK

SEQ ID NO: 6 - protein, 30 aa, rat
ETTEDCIAKIMNGEADAMSLDGGFVYIAGK

SEQ ID NO: 7 - protein, 19 aa, rat
INHCRFDEFFSEGCAPGSK

SEQ ID NO: 8 - protein, 22 aa, rat
KDSSLCKLCMGSGLNLCEPNNK

SEQ ID NO: 9 - protein, 18 aa, rat
NLNEKDYELLCLDGTRKP

SEQ ID NO: 10 - protein, 30 aa, rat; identical to SEQ ID NO: 6

SEQ ID NO: 11 - protein, 40 aa, rat
SVGKIECVSAETTEDCIAKIMNGEADAMSLDGGFVYIAGK

SEQ ID NO: 12 - DNA, 34 bp, artificial
  PCR primer
atgcccgcggatgaggctcaccgtgggtgccctg

SEQ ID NO: 13 - DNA, 45 bp, artificial
  PCR primer
gcatgcggccgcaattaatgtttgtggaaagtgcaggcttccagg

(19) United States
(12) Patent Application Publication
(10) Pub. No.: US 2022/0120764 A1
(43) Pub. Date: Apr. 21, 2022

(54) NOVEL BIOMARKER FOR ALZHEIMER'S DISEASE IN HUMAN

(71) Applicant: University of Manitoba, Winnipeg (CA)

(72) Inventor: Mohammad Golam Sabbir, Winnipeg (CA)

(21) Appl. No.: 17/282,651

(22) PCT Filed: Oct. 3, 2019
(86) PCT No.: PCT/CA2019/051417

§ 371 Date: Apr. 2, 2021

Related U.S. Application Data
(60) Provisional application No. 62/741,148, filed on Oct. 4, 2018.

(51) Int. Cl. G01N 33/68

(52) U.S. Cl. CPC G01N 33/6896; G01N 2440/14; G01N 2800/2821; G01N 2800/2814; G01N 2800/50

(57) ABSTRACT

The present invention relates to an in vitro method for determining the risk of developing Alzheimer's disease or a cognitive disorder similar to said disease, an in vitro method for designing a personalized therapy in a subject suffering from mild cognitive impairment and an in vitro method for selecting a patient susceptible to be treated with a therapy for the prevention and/or treatment of Alzheimer's or a cognitive disorder similar to said disease based on determining, in a sample from the subject, the level of phosphorylation in serine, tyrosine and/or threonine residues of interest in transferrin protein or in a functionally equivalent variant. The invention also relates to the use of transferrin protein or a functionally equivalent variant thereof, wherein the transferrin protein or variant is phosphorylated as a marker of the risk of developing Alzheimer's disease or a cognitive disorder similar to Alzheimer's disease. Finally, the invention relates to a kit comprising a reagent capable of determining the level of phosphorylation in residues of interest of transferrin protein and the use of said kit.

### FIELD OF THE INVENTION

The present invention relates to methods for determining the risk of developing Alzheimer, and to methods, and uses and kits thereof to detect phosphorylated transferrin protein in a subject at risk of developing Alzheimer or cognitive disorders related thereto.

### BACKGROUND OF THE INVENTION

Calcium ions (Ca²⁺) are one of the most ubiquitous second messengers and play crucial roles in many signaling pathways, especially in the neuronal tissues (Kawamoto et al., 2012). Calmodulin (calcium-modulated protein: CaM) is an intracellular Ca²⁺ receptor, able to bind four Ca²⁺ ions (Swulius and Waxham, 2008). The Calcium/Calmodulin-Dependent Protein Kinase Kinase 2 (CaMKK2) is a serine/threonine (Ser/Thr) kinase that is activated by Ca²⁺ and CaM binding (Racioppi and Means, 2012). Active CaMKK2 subsequently phosphorylate and activate three major downstream kinases, CaMKI, CaMKIV and AMPK respectively (Marcelo et al., 2016), which leads to regulation of cell growth as observed in neurite elongation and branching (Wayman et al., 2004), cell cycle control (Kahl and Means, 2004), energy balance (Anderson et al., 2008; Lin et al., 2011; Anderson et al., 2012), and gene expression and protein synthesis (Oury et al., 2010; Lin et al., 2015). CaMKK2 is expressed ubiquitously and has its strongest expression in the human brain (Uhlen et al., 2015). Dysregulation of CaMKK2 is strongly associated with a number of human diseases including neurodegeneration and cancer (Uhlen et al., 2017). In order to understand the role of CaMKK2 in neurons, we knocked down CaMKK2 in cultured primary adult rat dorsal root ganglion (DRG) neurons and profiled total cellular proteins based on net the electrical charge (isoelectric point: pI) and molecular weight. Protein profiling followed by a mass spectrometric study of differentially charged proteins identified reduced phosphorylation of transferrin (P-TF) at multiple amino acid residues in the CaMKK2 knockdown DRG neurons.

Transferrin (TF) is an iron transporter glycoprotein. Iron is an integral part of the haem and iron-sulfur (Fe—S) cluster and acts as a co-factor for numerous key enzymes involved in metabolic reactions (Rouault, 2013). Free iron can promote free radical formation resulting in oxidative damage (Gomme et al., 2005). Therefore, iron is transported safely in a redox-inactive state by TF. Circulating TF captures iron released into the plasma mainly from intestinal enterocytes or reticuloendothelial macrophages (Abbaspour et al., 2014) which then binds to the cell-surface TF receptor (TFR) and is internalized (Gomme et al., 2005). The internalized iron may be donated to cytosolic target proteins through chaperons (Philpott, 2012), or trafficked to mitochondria for the synthesis of haem or Fe—S clusters (Barupala et al., 2016), or stored in cytosolic ferritin (Arosio et al., 2009). Dysregulation of iron metabolism contributes to various human pathologies, including iron overload diseases (Fleming and Ponka, 2012), neurodegenerative brain diseases (Rouault, 2013), atherosclerosis (Sullivan, 1981) and cancer (Bogdan et al., 2016).

Dysfunction of CaMKK2 and upstream kinases, cyclin-dependent kinase 5 (CDKS) and glycogen synthase kinase 3 (GSK3) (Green et al., 2011), have been implicated in diverse human diseases, including Alzheimer's disease (AD), Parkinson's disease (PD), type 2 diabetes, bipolar disease and cancer (Frame and Cohen, 2001; Kockeritz et al., 2006). CDKS is a neuron-specific kinase that has been linked to an array of neurodegenerative disorders including AD, PD and Huntington's disease (Cheung and Ip, 2012; Kawauchi, 2014). In addition, decline in cAMP-dependent protein kinase (PKA) signaling, another CaMKK2 upstream kinase (Wayman et al., 1997; Cao et al., 2011), contributes to the etiology of several neurodegenerative diseases, including AD and PD (Dagda and Das Banerjee, 2015). The iron levels generally increase in an aging brain (Bartzokis et al., 1997) but in AD and PD, brain iron content shows a dramatic increase (Altamura and Muckenthaler, 2009). In AD, iron accumulates in the same brain regions that are characterized by the hallmark amyloid-β peptide (Aβ) deposition, such as the hippocampus, parietal cortex and motor cortex (Dedman et al., 1992; Good et al., 1992). In PD, one of the pathological hallmarks is neurodegeneration with brain iron accumulation and diffuse Lewy body formation (Altamura and Muckenthaler, 2009). The Lewy bodies are mainly composed of α-synuclein protein aggregates (Goedert, 2001) and multiple studies have now shown that iron promotes the aggregation of α-synuclein (Hashimoto et al., 1999; Golts et al., 2002). While the reasons for brain iron accumulation in these disorders are unknown, it correlates with the production of ROS and oxidative damage that hallmark these neurodegenerative disorders (Altamura and Muckenthaler, 2009). Interestingly, a genome-wide analysis of human kinases involved in endocytosis revealed that silencing of CaMKK2 isoform-1 in HeLa cells leads to decreased accumulation of fluorescent-TF in enlarged cytoplasmic structures which indicates defective TF trafficking (Pelkmans et al., 2005).

### SUMMARY OF THE INVENTION

According to a first aspect of the invention, there is provided a method for screening an individual who is at risk of dementia for dementia diagnosis comprising:
providing a sample from the individual; measuring a level of at least one isoelectric point fraction of transferrin in the sample; and comparing the sample level of the at least one isoelectric point fraction to a control level of the at least one isoelectric point fraction from a healthy individual, wherein for a positive result, the sample level and the control level are different.

According to another aspect of the invention, there is provided a method for screening an individual who is at risk of dementia for dementia diagnosis comprising:
Providing a sample from the individual; determining a transferrin profile of the sample; and comparing the transferrin profile of the sample to a reference value from a healthy individual, wherein for a positive result, the transferrin profile of the sample and the reference value are different.

In a first aspect, the invention relates to an in vitro method for determining the risk of developing Alzheimer's disease or a cognitive disorder similar to the said disease in a subject, which method comprises
a) Determining in a sample from the subject the level of phosphorylation in residues of interest in transferrin protein or in a functionally equivalent variant and
b) Comparing the level of phosphorylation obtained in a) to a reference value,

wherein an increase in the level of phosphorylation in residues of interest in transferrin protein or in a functionally equivalent variant compared to a reference value is indicative that said subject has a high risk of developing Alzheimer's or a cognitive disorder similar to the said disease.

In a second aspect, the invention relates to an in vitro method for designing a personalized therapy in a subject suffering from mild cognitive impairment, which method comprises
a) determining in a sample from the subject the level of phosphorylation in residues of interest in transferrin protein or in a functionally equivalent variant and
b) comparing the level of phosphorylation obtained in a) to a reference value,
   wherein an increase in the level of phosphorylation in residues of interest in transferrin protein or in a functionally equivalent variant compared to the reference value is indicative that said subject is susceptible to receive a therapy for the prevention and/or treatment of Alzheimer's disease or a cognitive disorder similar to the said disease.

In a third aspect, the invention relates to an in vitro method for selecting a patient susceptible to be treated with a therapy for the prevention and/or treatment of Alzheimer's or a cognitive disorder similar to the said disease, which method comprises
a) determining in a sample from the subject the level of phosphorylation in residues of interest in transferrin protein or in a functionally equivalent variant and
b) comparing the level of phosphorylation obtained in a) to a reference value,
   wherein an increase in the level of phosphorylation in residues of interest in transferrin protein or in a functionally equivalent variant compared to the reference value is indicative that said subject is a candidate for receiving a therapy for the prevention and/or treatment of Alzheimer's disease or a cognitive disorder similar to the said disease.

In a fourth aspect, the invention relates to the use of transferrin or a functionally equivalent variant thereof, wherein the transferrin or variant is phosphorylated in a residue of interest as a marker of the risk of developing Alzheimer's disease or a cognitive disorder similar to Alzheimer's disease.

In a fifth aspect, the invention relates to a kit comprising a reagent capable of determining the level of phosphorylation in residues of interest of transferrin protein for determining the risk of a subject developing Alzheimer's or a cognitive disorder similar to Alzheimer's disease, for designing a personalized therapy in a subject or for selecting a patient susceptible to be treated with a therapy for the prevention and/or treatment of Alzheimer's disease or a cognitive disorder similar to Alzheimer's disease.

In a sixth aspect, the invention relates to the use of a kit according to the invention for determining the risk of a subject developing Alzheimer's disease or a cognitive disorder similar to said disease in a subject, for designing a personalized therapy in a subject suffering from mild cognitive impairment or for selecting a patient susceptible to be treated with a therapy for the prevention and/or treatment of Alzheimer's or a cognitive disorder similar to the said disease.

### BRIEF DESCRIPTION OF THE DRAWINGS

FIG. 1: Protein profiling in CaMKK2 knockdown cultured adult primary rat DRG neurons. A: Diagrammatic representation of the CaMKK2 gene structure showing location and sequence of 3 siRNAs used to knockdown CaMKK2. Exons are demarcated by vertical lines. B: Immunoblots showing expression of CaMKK2, TF and GAPDH. CTRL: scrambled control, KD: knockdown. The LNP based delivery of siRNAs knocked down 98% of CaMKK2 in DRG neurons. C: Oriole stained IEF/SDS-PAGE gel showing focused proteins. Detailed methodology is described in text. Green and blue rectangle marked area showing marked difference in the charged protein fractions. The gel images were false colored and overlaid in D to highlight the differences. The roman numerals indicate gel spots that were used for in-gel trypsin digestion and mass spectrometric identification of the proteins. F: Table summarizes mass spectrometry findings. log(e): the base-IO log of the expectation that any particular protein assignment was made at random (E-value).

FIG. 2: Reduced P-TF (pH-3-4 fraction) in CaMKK2 knockdown DRG neurons. A: Immunoblots showing relative expression of CaMKK2, GAPDH and TF in CaMKK2 knockdown DRG neurons. CTRL: scrambled control, KD: knockdown. B: Immunoblots showing charged fractions of TF in DRG neurons. Red rectangles indicate pH/pI˜3, ˜5-6 and ˜9-10 fractions of native TF. Blue rectangles indicate higher molecular weight form of TF which may be due to post translational modifications that impart mass. C: Scatter plot showing relative percentage of P-TF in CaMKK2 knockdown cells. The percentage was calculated relative to the pH-9-10 fraction. N=6 from 3 independent experiments. P value by t-test (unpaired).

FIG. 3: Reduced abundance and altered phosphorylation of TF in CaMKK2 knockout mouse DRG tissues. A: TF promoter-trapped GFP reporter expression in adult, postnatal (P7) and embryonic 15.5 stage spinal cord and DRGs (founder line: IF181). Top panel: GFP-immunostained paraffin embedded sections. Bottom panel: GFP epifluorescence in cryomicrotome sections. The images were obtained from GENSAT project, Rockefeller University, New York, USA. B: Immunoblots showing expression of CaMKK2, TF, and ERK1/2 in adult mouse DRG tissues respectively. C: Scatter plot showing relative amount of TF (normalized to ERK1/2) in DRG tissues. N=8 replicates from 4 mice in each category, p value by t-test (unpaired). D: Immunoblots showing charged fractions of TF in DRG tissues. Rectangle area represents altered charge of TF at different pHs. Red and black arrows indicate difference in TF charged fractions. The bottom 2 panels represent immunoblots from 2 CaMKK2 KO mice. E: Superimposed line graph showing relative intensity vs pixel distance obtained from the rectangle area of the 3 immunoblots presented in D. The 3 spots are numbered in Arabic numerals. Black arrow indicates significant change. F: Scatter plot showing the relative intensity of peak “2” in E. Peak 2 is completely absent in the CaMKK2 KO mice. Red arrows indicate TF pH˜3 fractions shifted to less acidic pH.

FIG. 4: Increased abundance and reduced phosphorylation of TF in CaMKK2 KO cerebellum and olfactory bulb tissues. A: TF promoter-trapped GFP reporter expression in adult brain tissues (founder line: IF18 1). Top panel: GFP immunostained paraffin embedded sections. Bottom panel: GFP epifluorescence in cryomicrotome sections. The images were obtained from GENSAT project, Rockefeller University, New York, USA. B &F: Immunoblots showing expression of CaMKK2, GAPDH, TF, nucleolin (B23) and VDACI in adult mouse olfactory bulb and cerebellum tissues. C&G: Scatter plot showing relative amount of TF normalized to GAPDH/B23. N=10/5 replicates from 3 KO and wild type mice respectively, p value by t-test (unpaired). D&H: Immunoblots showing charged fractions (PTMs) of TF. Rectangle area represents altered charge of TF at different pHs. Blue rectangle indicates PTMs that imparted additional mass. E&I: Scatter plot showing relative amount of TF pH-3 fractions. N=2 replicates from 3 KO and wild type mice respectively, p value by t-test (unpaired). The intensities in the pH˜3 fractions were normalized as percentage of the pH-10 fractions (red dotted rectangle) and plotted in E.

FIG. 5: Abundance and phosphorylation of TF in CaMKK2 KO cerebral cortex and liver tissues. A & E: Immunoblots showing expression of CaMKK2, TF, VDAC1, and Histone-1(H1). B&F: Scatter plot showing relative amount of TF (normalized to VDAC1/H1 respectively). N=8/6 replicates from 3 CaMKK2 KO and wild type mice respectively. C&G: Immunoblots showing charged fractions of TF. Rectangle area represents altered charge of TF at different pHs. Each blot represents individual mouse. D&H: Scatter plot showing relative amount of TF pH-3-4 fractions. N=8/9 replicates from 3 KO and wild type mice respectively. The intensities in the TF pH-3-4 fractions (red rectangle) were normalized as percentage of the pH-5-6 fractions (black/green rectangle) and plotted. P values by t-test (unpaired).

FIG. 6: Abnormal intracellular vesicular trafficking of TF in CaMKK2 knockdown DRG neurons: A-B: Live confocal images showing intracellular localization of cell permeant TMR-ligand labelled Halo-TF in cultured DRG neurons. Halo-TF was expressed in scrambled control and CaMKK2 knock down cultured adult rat primary DRG neurons for 48 hours and labelled with cell permeant Halo-TMR reagent and live imaged. The knockdown efficiency was checked by immunoblotting in a parallel experiment. Halo-TMR covalently attached to the Halo-tag through a chloroalkane (reactive linker) group to the Phe272 residue in Halo-Tag. White arrows indicate neurites and blue rectangles indicate perikaryon. The perikaryon were imaged at higher magnification and shown in the right panel. Z: optical slice in Z-dimension. C: Threshold adjusted and despeckled images from marked areas in B. The TMR-Halo-TF positive vesicular particles (blue arrow) were quantified using ImageJ automated particle counting plugin (Sbalzarini and Koumoutsakos, 2005). The parameters were as follows: circularity-0-1 (0=infinitely elongated polygon to 1=perfect circle), particle size 50-50,000 pixels. Scale bar-504. D: Whisker plot showing number of particles counted in the perikaryon/neuron. N=50 replicates from 2 independent studies, p value by t-test (unpaired). E: Binning of the data presented in D. F: Immunofluorescence images showing colocalization of TRM-Halo-TF and Rab5/Rab11 in cultured DRG neurons. The small GTPase-Rab5 is an early endosomal vesicle specific marker and Rab11 is mainly associated with recycling endosomes (Mills et al., 2010). Scale bar-504. White arrow indicates vesicular structure.

FIG. 7: Altered P-CaMKK2 and P-TF in 3xTg-AD mice. A: Immunoblot showing charged fractions of CaMKK2 isoform 1 and 2 in 6 months' female wild type and 3xTg-AD mice. Colored arrows indicate different charged fractions. Linear pH 4-7 IPG strips were used to resolve closely spaced CaMKK2 charged fractions. B: Plot profile showing relative intensity of the focused CaMKK2 isoform-1 spots. C: Scatter plot showing relative percentage of the comparatively more negative charged fraction (red arrow) of CaMKK2 isoform-1. The spots marked with black arrows were used for normalization. D-E: Immunoblots showing charged fractions of TF. Colored rectangle indicates different charged fractions. F-G: Scatter plot showing relative intensities of the pH-3 fractions of TF. The intensities of pH-5-6 fractions were used for normalization. N=6 replicates from 3 mice in each categories. P values by t-test (unpaired).

FIG. 8: Altered TF and CaMKK2 associated protein complexes in 3xTg-AD. A-B: Immunoblots showing TF and CaMKK2 associated protein complexes in DRG, cerebral cortex and DRG tissues respectively. Dotted vertical lines indicate vertical alignment of co-migrated protein complexes. Colored circles indicated different protein complex, ns: non-specific. The coomassie stained gel strip on the top panel showing native page molecular weight ladder. C: Scatter plot showing relative intensities of ˜1000 kDa TF associated protein complex. The ˜720 kda complex was used for normalization. D: Immunoblot showing TF level in serum. Bottom panels: SOS-PAGE gel stained with Oriole to show total proteins loading. E: Immunoblots showing charged fractions of TF in serum. Red dotted rectangle indicates negative charged fractions of TF. Blue dotted rectangles indicate high molecular weight TF PTMs. F: Scatter plot showing relative abundance of P-TF in serum. The pH-3 fractions were normalized on the basis of pH-6 fractions intensities (blue rectangle). N=6 replicates from 3 mice in all experiments. P values by t-test (unpaired).

FIG. 9: Relative abundance and phosphorylation of TF in the serum samples obtained from early and late 3xTg-AD and age matched control mice. A & D: Top Panels: Immunoblots showing TF level in serum. Bottom panels: SOS-PAGE gel stained with Oriole to show total protein loading. Black arrows indicate the band used for normalization of TF amount. B: Scatter plot showing relative abundance of TF in serum. N=8 (2 replicates from 4 mice in each category). P values by t-test (unpaired). C & F: Immunoblots showing charged fractions of TF. Red dotted rectangle in C indicates negative charged fractions of TF. Colored dotted rectangles in F indicate different charged fractions of TF.

FIG. 10: Relative abundance and phosphorylation of TF in the CSFs and matched serums obtained from postmortem human EOAD. A&B: Top Panels: Immunoblots showing expression of TF in CSF and matched serum. Bottom panels: SDS-PAGE gel stained with Oriole to show total protein loading. CSFs (15 μl) and serums (3 μl) were loaded in each lane respectively. Black arrow indicates the band used for normalization of TF expression. C: Scatter plot showing relative abundance of TF N=4 (2 replicates for 2 samples). P values by t-test (unpaired). D &E: Immunoblots showing charged fractions of TF. Red dotted rectangle indicating negative charged fractions of Tf. F: Plot profiles showing the relative intensities of focused spots in the immunoblots in E. G: Table showing detailed patient information.

FIG. 11: Relative abundance and phosphorylation of TF in the CSFs obtained from postmortem human EOAD. A: Top Panel: Immunoblots showing expression of TF in the CSFs. Bottom panels: SDS-PAGE gel stained with Oriole to show total protein loading. CSFs (15 μI) and serums (3 μl) were loaded in each lane respectively. Black arrow indicates the band used for normalization of TF expression in CSFs and serums. B: Scatter plot showing relative abundance of TF in EOAD CSFs and matched serum samples. N=2/3 (2 replicates in each category). P values by t-test (unpaired). C: Immunoblots showing charged fractions of TF. Red dotted rectangle indicates negative charged fractions of TF. D: Plot profiles showing the relative intensities of focused spots in the immunoblots in C and FIG. 10D. Grey rectangle is showing loss of P-TF in AD (except 2 samples). E: Table showing detailed patient information.

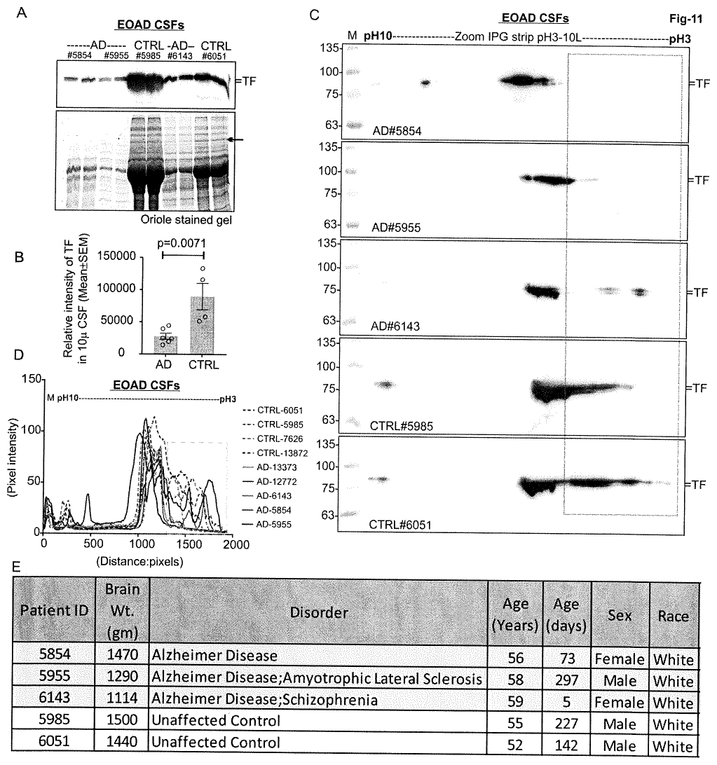

FIG. 12: Relative abundance and phosphorylation of TF in the CSFs obtained from postmortem human LOAD. A&B: Top Panels: Immunoblots showing expression of TF in CSF and matched serum. Bottom panels: SDS-PAGE gel stained with Oriole to show total protein loading. CSFs (15 μI) and serums (3 μl) were loaded in each lane respectively. C &D: immunoblots showing charged fractions of TF. Red dotted rectangle indicates negative charged fractions of TF. Green and blue rectangles show high molecular weight fractions of TF. E: Table showing detailed patient information.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

We hypothesized that CaMKK2 controls TF phosphorylation and intracellular trafficking and that aberrant CaMKK2 may imbalance TF abundance and phosphorylation during development and progression of AD. Therefore, we studied relative expression, charged fractions (by isoelectric focusing: IEF), and intracellular trafficking of TF in vivo and in vitro using CaMKK2 knockout (KO) mice, CRISPR/Cas9 based CaMKK2 knockout human cell lines (HEK293 and HepG2) and a siRNA based knockdown approach. In addition, we analyzed CaMKK2 and TF charged fractions and TF associated protein complexes in the 3xTg-AD mice hippocampus and cortical tissues during an early and late stage of AD development. The 3xTg mouse-based study indicated that aberrant CaMKK2 during AD development/progression resulted in a significantly reduced amount of P-TF (which corresponds to an acidic fraction of transferrin, in some instances, corresponding to a charged fraction of pH 3-4, as discussed herein) in the brain. The implication of this finding is immense because it indicates that secreted P-TF from the diseased organ into the serum and/or cerebrospinal fluid (CSF) can be exploited as an invasive (CSF) or minimal-invasive (serum) biomarker for diagnosis and prognosis of AD in the human.

We analyzed the abundance and charged status of TF in the CSF and serum samples obtained from postmortem human early and late-onset AD (EOAD and LOAD respectively) patients as well as 3xTg-AD mouse (serum only). Our findings indicate that aberrant CaMKK2 phosphorylation leads to a significant decrease in P-TF possibly due to impaired vesicular trafficking. The hallmark phosphorylated TF fractions in the serum and the CSF were successfully used to identify EOAD and LOAD in human postmortem patients. Our study indicates a novel CaMKK2 mediated TF-signaling pathway. We provide evidence that phosphorylated TF (pH-3-4 fraction) can be used as a novel diagnostic and prognostic biomarker for AD in human.

Specifically, in this study, we report that loss of CaMKK2 significantly reduced phosphorylation of TF at multiple Ser/Tyr/Thr residues in neuronal cells. The P-TF residues were identified in pH-3-4 fraction by mass spectrometry. In the subsequent studies, we described a pH-3-4 fraction of TF as a measure for the P-TF level. Our study indicates that CaMKK2 is directly or indirectly responsible for TF phosphorylation and turnover in a tissue-specific manner in vivo. In addition, we have shown that knockdown of CaMKK2 leads to aberrant vesicular trafficking of TF which may account for its reduced turnover. Further, we have shown a possible association of CaMKK2 and TF in multiple protein complexes and their dynamics in vivo. In addition, we provide a novel mechanism linking dysregulated CaMKK2 and aberrant transferrin phosphorylation and turnover in 3xTg-AD mouse-based study which may provide a novel insight into the mechanism for neurodegeneration. We also evaluated PTF (pH˜3-4 fraction) as a potential CSF and serum-based prognostic and diagnostic biomarker for AD in humans. This has immense implications in early diagnosis, treatment and patient care in AD.

The function of potential novel P-TF residues are not known but bioinformatics analysis indicated some important features. The majority of the P-TF residues have surface accessibility which indicates functional relevance. The Tyr257/338, Ser381/389/409/511, and Thr586 residues are conserved between different mammalian species. The Ser381/389 and Thr392/393 residues are positioned within the conserved TFR binding site identified by multiple techniques including cryo-electron microscopy (residues 349-372) (Cheng et al., 2004), radiation footprinting (residues 381-401) (Liu et al., 2003; Xu et al., 2005) and epitope mapping based studies (residues 365-401) (Teh et al., 2005). Loss of phosphorylation at S-381/389 and 1392/393 may affect the interaction of TF with TRF (Wally et al., 2006). PTF(T392/393) residues have been detected in the mass spectrometric analysis of cytoskeleton-associated proteome in HeLa cells (GPM ID: GPM70110006894) (Ozlu et al., 2010). The Ser409 residue is conserved in different mammalian species and located in close proximity to one of the iron binding residue (Asp411) in the C-lobe of TF (Yang et al., 2012). A high stringency protein motifs search by the ScanSite “MotifScan” module function (Obenauer et al., 2003) revealed a plurality of short motifs putatively concerned with protein interactions and signal transduction molecules, including two SH2 target sequences, a tyrosine kinase, three acidophilic kinase, a Ser/Thr kinase, and a proline-dependent Ser/Thr kinase binding sites, as well as consensus binding sites for 3-Phosphoinositide-dependent Protein Kinase 1 (PDPK1), overlapped the P-TF residues. For example, Tyr333/336/338 residues overlapped with a potential PDPK1 binding motif. The P-TF (Tyr333) residue in PDPK1 binding motif (³³²MYₚLGYEYVTAIR³⁴³, GPM ID: GPM323 1 0000046) has been previously detected in the proteomic analysis of human CSF (Chiasserini et al., 2014). This motif is highly conserved among different vertebrate species. PDPK1 is a master serine/threonine kinase that phosphorylates AGC family of protein kinases (cAMP-dependent, cGMP-dependent and protein kinase C) (Alessi et al., 1 997; Chou et al., 1998; Manning and Cantley, 2007). Pelkmans et al. used high throughput RNA interference combined with infectious virus (vesicular stomatitis virus, VSV) entry and florescent-TF uptake assay to study the human kinases associated with clathrin- and caveolae/raft-mediated endocytosis (Pelkmans et al., 2005). Interestingly, they found that silencing CaMKK2 isoform-1 in HeLa cells led to the decreased accumulation of fluorescent-TF in enlarged cytoplasmic structures (Pelkmans et al., 2005). In the same study, silencing PDPK1 resulted in comparatively lower VSV infection and exhibited a toxic effect in response to TF uptake (Pelkmans et al., 2005). This indicates a possible link between PDPK1 and CaMKK2 in regulating TF uptake or trafficking.

CaMKK2 is widely expressed in the neurons of rodent CNS and PNS (Ohmstede et al., 1989; Anderson et al., 1998; Sakagami et al., 1998; Vinet et al., 2003). A TF promoter-trapped GFP epifluorescence study showed expression of TF in the neurons of mouse CNS and PNS. The overlapping expression pattern of CaMKK2 and TF is also conserved in human nervous system and liver tissues which indicates that dysfunctional CaMKK2 may directly impact TF abundance and phosphorylation which in turn may alter brain iron levels or TF mediated signaling. Cells in the nervous system do not have direct access to nutrients, including iron (Rouault, 2013). TF secreted by the liver in the serum function to deliver iron to different organs including brain (Sawaya and Schaeffer, 1995). TF and TFR mediate uptake of iron across the blood-brain-barrier (Brightman et al., 1970; Ballabh et al., 2004). The generally agreed mechanism is that the brain capillary endothelial cells absorb iron-bound TF (holo-TF) from the blood via TFR mediated endocytosis (Bradbury, 1997). After endocytosis, the acidic environment of the early endosomes triggers the release of Fe³⁺ from the TF-TFR complex, which is recycled to the plasma membrane via recycling endosomes (Mills et al., 2010). How iron subsequently exits the brain capillary endothelial cells and reaches the brain extracellular fluid is less well understood (Altamura and Muckenthaler, 2009). Within the brain interstitium, iron is transported bound to low molecular weight constituents like ATP and citrate (Bradbury, 1997) as well as large molecules like TF and lactoferrin (Fillebeen et al., 1999). TF and non-TF bound iron are taken up by neurons, astrocytes and oligodendrocytes (Ke and Qian, 2007). Our findings that knockdown of CaMKK2 led to loss of TF enriched endosomal vesicles indicate that intracellular TF-trafficking may be perturbed due to CaMKK2 dysfunction. This in turn may lead to abnormal iron deposition and subsequent neurodegeneration.

High iron concentration in the brain and mutations in the genes associated with iron metabolism were found in neurodegenerative disorders like AD, Parkinson's and Huntington's disease which suggest that iron misregulation in the brain plays a part in neuronal death (Ke and Ming Qian, 2003). Histological analysis revealed that aberrant homogenous and extracellular TF is distributed around the senile plaques in human AD brain tissues which also stained positive for iron accumulation (Connor et al., 1992). A splicing defect in the TF gene resulted in <1% of the normal plasma levels of TF in hypotransferrinemic (HP) mice (Takeda et al., 2002). Brain iron concentration in the HP mouse was found to be significantly high in the cerebellar cortex (3×), cerebellum (Takeda et al., 2001), bone marrow (Takeda et al., 2002) and liver (100×) (Trenor et al., 2000). Histological analysis of HP mutant mouse revealed a decreased amount of white matter and altered neuronal morphology throughout the brain (specifically hippocampus and cerebellum) and the spinal cord (Dickinson and Connor, 1994). In Parkinson's disease (PD), one of the pathological hallmarks is neurodegeneration with brain iron accumulation and diffuse Lewy body formation (Altamura and Muckenthaler, 2009). The Lewy bodies are mainly composed of α-synuclein protein aggregates (Goedert, 2001) and multiple studies have now shown that iron promotes the aggregation of α-synuclein (Hashimoto et al., 1999; Golts et al., 2002). In PD, the olfactory bulb is typically the first region in the body to accumulate α-synuclein aggregates which is linked to decreased olfactory ability. A recent study using human subjects showed significantly increased iron in the olfactory bulb which correlated to olfactory dysfunction (Gardner et al., 2017). Friedreich's ataxia is an autosomal recessive neurodegenerative disease resulting from mutations in the mitochondrial protein frataxin (Campuzano et al., 1996) or a defective phosphatidylinositol-4-phosphate 5-kinase, known as STMT (Carvajal et al., 1996). The disease is characterized by degeneration of large sensory neurons, brain atrophy and iron accumulation (Gordon, 2000). Frataxin acts as an iron storage protein in the mitochondria (Puccio et al., 2001; Richardson et al., 2010) and defects in STMT affect vesicular trafficking (Carvajal et al., 1996), both linked to dysregulation of intracellular iron metabolism. Neurodegeneration with brain iron accumulation (NBIA) is a group of neurodegenerative diseases characterized by most severe iron accumulation in the cerebellum and cerebellar atrophy (reduction in Purkinje cells) in some subtypes (Gregory and Hayflick, 1993; Levi and Finazzi, 2014; Arber et al., 2016). Purkinje cell death is most pronounced in the learner mutant mouse which carries an autosomal recessive mutation in the gene coding for the α1A pore forming subunit of the CaV2.1P/Q-type voltage-gated calcium channel (Herrup and Wilczynski, 1982). CaMKK2 knockout mice exhibited morphological and physiological deficits in Purkinje cells (Ribar et al., 2000; Kokubo et al., 2009). Dysregulation of the intracellular Ca²⁺ homeostasis was suggested to underlie the development of Alzheimer's disease (AD) (Hermes et al., 2010; Berridge, 2011). Hyperphosphorylation of tau and Aβ plaques are pathological hallmarks of AD (Ballatore et al., 2007; Serranno-Pozo et al., 2011). It has been shown that Aβ peptides (Aβ1-42) alters intracellular Ca²⁺ through NMDA receptors in mouse cortical neurons which in turn lead to phosphorylation of tau by AMPK through CaMKK2-AMPK signaling pathway (Thornton et al., 2011; Mairet-Coello et al., 2013). In our study, the amount of TF significantly increased in the olfactory bulb and cerebellum of CaMKK2 KO mice which may explain why olfaction (Esiri and Wilcock, 1984; Kovacs et al., 2001; Zou et al., 2016), voluntary motor activity and motor learning (Guo et al., 2016; Jacobs et al., 2018) are pathologically affected in early stages of AD. A significant decrease of TF in the CaMKK2 KO liver also indicates possible link with the recent findings that liver may play role in the development of AD. Presenilin 2 (Psen2) is a component of the γ-secretase activity responsible for generating Aβ by proteolysis. It has been shown that Psen2 mRNA accumulation is heritable in the liver but not in the brain, suggesting liver as the origin of brain Aβ (Sutcliffe et al., 2011). Chronic liver disease, like non-alcoholic fatty liver disease (NAFLD), induced by consumption of high-lipid diets and characterized by liver inflammation has been linked to increased risk of AD pathologies (Kim et al., 2016). In addition, shortage of the lipid docosahexaenoic acid (DHA) in AD brains, in the cortex and cerebellum, has been linked to defects in liver enzyme D-bifunctional protein (DBP), which catalyzes the final step of DHA biosynthesis from tetracosahexaenoic acid (Astarita et al., 2010; Astarita and Piomelli, 2011). Accordingly, we suggest that dysregulation of CaMKK2 in brain and liver may lead to aberrant TF signaling which may imbalance iron homeostasis in the brain and cause disease.

Our study raises an important question. Does CaMKK2 directly phosphorylate TF? Mass spectrometric analysis of interacting proteins associated with CaMKK2 & TF protein complexes are important to identify the specific calmodulin component of such kinase activity. We have attempted to answer this question in an indirect way by performing BN-PAGE analysis. The co-migration of CaMKK2 isoform-2 and TF associated protein complexes indicates possible direct protein-protein interaction. In addition, the disappearance of ˜242 and 100 kDa TF complexes in CaMKK2 KO DRG tissues, which are not vertically aligned with CaMKK2 protein complexes in BN-P AGE, indicates that other CaMKK2 dependent interacting proteins (kinases) may be involved. The identification of potential P-TF (Tyr257/338) supports that conclusion. Further, the relative difference in the molecular weight of TF associated protein complexes in different tissues indicates different interacting proteins and different TF signaling pathways operate in different tissues. Alternative splicing of exons 14 and/or 16 and usage of differential polyadenylation sites generates several transcriptional isoforms of CaMKK2 that are expressed in a tissue specific manner (Hsu et al., 2001). The CaMKK2 (+16) transcript is highly enriched in cerebellum and hippocampus (Ohmstede et al., 1989). Over expression of (+16)-transcript leads to neurite branching whereas (Δ16)-transcript promoted neurite elongation (Cao et al., 2011). Emerging evidence suggest that altered neurogenesis in the adult hippocampus represents an early event in the course of AD (Mu and Gage, 2011). The Δ14/16 CaMKK2 isoforms lead to loss of detectable kinase activity towards CaMKI and CaMKIV, whereas full length CaMKK2 isoforms exhibited kinase activity to both effectors as well as autophosphorylation activity (Hsu et al., 2001). CaMKK2 has been implicated in cAMP-responsive element binding protein (CREB) activation and memory consolidation process in the hippocampus (Soderling, 1999; Corcoran and Means, 2001; Soderling and Stull, 2001; Wayman et al., 2008). Loss of CaMKK2 affected formation of hippocampus dependent long-term memory (Peters et al., 2003). Therefore, reduced P-CaMKK2 isoform-1 in 3xTg-AD mouse cortex may indicate dysfunction of the protein which may affect neurite branching and memory consolidation process. Based on this study, it may be concluded that altered physiological state in AD brain led to reduced P-CaMKK2 which is responsible for reduced P-TF that alters iron homeostasis and eventually led to neurodegeneration.

AD is one of the most prevalent dementia worldwide (Sharma and Singh, 2016). The current diagnostic criteria for AD has included CSF biomarkers (Aβ peptides, Tau and P-Tau) which are obtained by invasive lumbar punctures that cause nausea, severe backache and weakness in elderly people (Lehmann and Teunissen, 2016). There is an urgent need for a minimally invasive serum-based diagnostic or prognostic biomarker that has significant advantages in time-efficiency and cost-efficiency as well as patient acceptance (Sharma and Singh, 2016; O'Bryant et al., 2017). Plasma proteins of amyloid pathology, circulatory miRNAs, cytokines, kinases, axonal proteins, lipids, and fragments of already known AD markers are currently investigated for their potential as blood-based AD biomarkers, see review by (Lista et al., 2013; Huynh and Mohan, 2017). In addition, serum TF level (Squitti et al., 2010), desaturation level of serum TF-iron (Hare et al., 2015), glycosylated-TF in CSF (Guevara et al., 1998; van Rensburg et al., 2000; Taniguchi et al., 2008; Shirotani et al., 2011) and serum (Yu et al., 2003) have been proposed as potential AD biomarkers. Our observation that TF levels in CSF of EOAD and LOAD patients significantly decreased validate previous findings. The serum TF level remained unaltered in CaMKK2 KO mice, 3xTg-AD mice and human patients which makes it unsuitable as a diagnostic biomarker. In addition, our findings that loss of CaMKK2 altered TF high molecular weight fractions which is an indication that glycosylated-TF (high molecular weight fraction) may reflect the diseased state of the brain in AD. However, use of glycosylated-TF as a diagnostic biomarker is a daunting analytical challenge due to inherent complexity and variability in glycans which is also highlighted in our study (Zhang et al., 2016). The consistent findings that loss of P-TF (pH˜3-4 fraction) in the serum of CaMKK2 KO mice, 3xTg-AD mice, and in the CSF and serum from EOAD and LOAD postmortem human patients indicate that TF phosphorylation is a promising novel AD prognostic and diagnostic marker.

Accordingly, in a first aspect, the invention relates to an in vitro method for determining the risk of developing dementia similar to said disease in a subject (first method of the invention), which method comprises
a) determining in a sample from the subject the level of phosphorylation in transferrin protein or in a functionally equivalent variant and
b) comparing the level of phosphorylation obtained in a) to a reference value, wherein an decrease in the level of phosphorylation of transferrin protein or m a functionally equivalent variant compared to a reference value is indicative that said subject has a high risk of developing dementia.

In a particular embodiment, the subject suffers from Alzheimer's or a cognitive disorder similar to said disease. In an alternate embodiment, the subject suffers from mild cognitive impairment.

The individual who is at risk of dementia may be an individual who displays one or more or more, sometimes two or more symptoms associated with dementia or has one or more risk factors associated with dementia. These symptoms and risk factors are well known in the art and can easily be determined by consulting a variety of sources.

For example, symptoms of dementia include but are by no means limited to: memory loss that disrupts daily life; challenges in planning or solving problems; difficult completing familiar tasks at home, at work or at leisure; confusion with time or place; trouble understanding visual images and spatial relationships; new problems with words in speaking or writing; misplacing things and losing the ability to retrace steps; decreased or poor judgement; withdrawal from work or social activities; and changes in mood and personality.

Risk factors of dementia include but are by no means limited to age, for example, if the individual is 65 or older; familial history, for example, if the individual has a close relative who had dementia; serious head injury, especially repeated trauma or if the trauma involves loss of consciousness; heart disease; diabetes; stroke; high blood pressure; and high cholesterol.

As it is used herein, “mild cognitive impairment”, also known as incipient dementia or isolated cognitive impairment refers to a nosologic entity that seeks to describe the symptomatology before the onset of dementia. Affected individuals suffer from impairments that are more advanced than expected for their age and level of education, but these impairments do not significantly interfere with their daily activities. It is considered as the limit between normal aging and dementia. The person skilled in the art is capable of identifying if a subject has a mild cognitive impairment based, for example, on the diagnostic criteria set forth in the Diagnostic and Statistical Manual of Mental Disorders (DSM) and in the International Classification of Diseases, which allow physicians to make their diagnoses.

In a particular embodiment of the invention, the subject is a human and the neurodegenerative disease is Alzheimer's disease.

As it is used herein, the expression “risk of developing” dementia, or Alzheimer's disease or a cognitive disorder similar to said disease refers to the predisposition, susceptibility, propensity or likelihood of a subject developing dementia, or Alzheimer's disease or a cognitive disorder similar to said disease. The risk of developing a neurodegenerative disease, dementia, Alzheimer's disease or a cognitive disorder similar to said disease generally means that there is a high or low risk or a higher or lower risk. Therefore, a subject with a high risk of developing dementia, or Alzheimer's disease or a cognitive disorder similar to said disease has a likelihood of developing said disease of at least 50, or at least 60%, or at least 70%, or at least 80%, or at least 90%, or at least 95%, or at least 97%, or at least 98%, or at least 99%, or at least 100%. Similarly, a subject with a low risk of developing dementia, Alzheimer's disease or a cognitive disorder similar to said disease is a subject having at least a likelihood of developing said disease of at least 0%, or at least 1%, or at least 2%, or at least 3%, or at least 5%, or at least 10%, or at least 20%, or at least 30%, or at least 40%, or at least 49%.

In general, the expression “predicting the risk”, “prediction of the risk” or the like, refers to the risk of a patient developing dementia, or Alzheimer's disease or a cognitive disorder similar to said disease, whether it is high or low. As will be understood by those skilled in the art, although the prediction (or risk) is preferable, it does not have to be correct for all the subjects to be evaluated, although it is preferable for it to be so. The term, however, requires a statistically significant part of the subjects being identified as exhibiting a higher likelihood of having a specific result. The person skilled in the art can determine without much difficulty if a part is statistically significant using different, well-known statistical evaluation tools, for example, the determination of confidence intervals, determination of p-value, cross-validation with classification indices, etc. Preferred confidence intervals are at least 50%, at least 60%, at least 70%, at least 80%, at least 90% or at least 95. p-values are preferably 0.1, 0.05, 0.02, 0.01 or less.

The term “Alzheimer's disease” or “AD” or “Alzheimer's” refers to a mental impairment associated with a specific degenerative brain disease which is characterized by the appearance of senile plaques, neurofibrillary tangles and progressive neuronal loss clinically manifested as progressive memory deficiencies, confusion, behavioral problems, inability to take care of oneself, gradual physical deterioration, and ultimately death. Alzheimer's disease can also be defined as a disease in any of the stages according to the Braak scale:
Stages I-II: the brain area affected by the presence of neurofibrillary tangles corresponds to the transentorhinal region of the brain
Stages the affected brain area also extends to areas of the limbic region, such as the hippocampus
Stages V-VI: the affected brain area also involves the neocortical region.

This classification by neuropathological stages is correlated with the clinical progression of the disease, there being parallelism between memory decline with neurofibrillary changes and the formation of neuritic plaques in the entorhinal cortex and hippocampus (stages I to IV). Likewise, the isocortical presence of these changes (stages V and VI) is correlated with clinically severe alterations. The transentorhinal stage (I-II) corresponds to clinically silent periods of the disease. The limbic stage (III-IV) corresponds to a clinically incipient AD. The neocortical stage corresponds to a fully developed AD. In addition, Alzheimer's can be defined as early onset Alzheimer's disease (EOAD) and late onset Alzheimer's disease (LOAD). EOAD occurs in about 5% of Alzheimer's patients who develop symptoms before age 65. Most of these patients have the sporadic form of the disease, but 10-15% have a genetic form that is generally inherited as an autosomal dominant fashion. LOAD is the most common form of the disease, which happens to people age 65 and older.

The present inventions may be applied to subjects who have not yet been diagnosed as having the respective diseases and conditions (for example, preventative screening), or who have been diagnosed as having such, or who are suspected of having such (for example, display one or more characteristic signs and/or symptoms), or who are at risk of developing such (for example, genetic predisposition; presence of one or more developmental, environmental or behavioral risk factors). The kits, methods and uses may also be used to detect various stages of progression or severity of the diseases and conditions. The kits, methods and uses may also be used to detect response of the diseases and conditions to prophylactic or therapeutic treatments or other interventions. The kits, methods and uses can furthermore be used to help the medical practitioner in deciding upon worsening, status-quo, partial recovery, or complete recovery of the subject from the diseases and conditions, resulting in either further treatment or observation or in discharge of the patient from a medical care center. Also, the test panels, methods and uses as taught herein may be employed for population screening, such as, e.g., screening in a general population or in a population stratified based on one or more criteria, e.g., age, ancestry, occupation, presence or absence of risk factors of the respective diseases and conditions, etc.

The respective quantities, measurements or scores for the biomarker(s) (e.g., phosphorylation of transferrin protein or in a functionally equivalent variant, antibodies and anti-antigens thereto, etc.) and parameter(s) (e.g., levels of biomarkers, age, extent of disease, reference value, etc.) in the present inventions may be evaluated separately and individually, i.e., each compared with its corresponding reference value. More advantageously, the quantities, measurements or scores for the biomarker(s) and parameter(s) may be used to establish a biomarker-and-parameter profile, which can be suitably compared with a corresponding multi-parameter reference value. In yet another alternative, the quantities, measurements or scores for the biomarker(s) and parameter(s) may each be modulated by an appropriate weighing factor and added up to yield a single value, which can then be suitably compared with a corresponding reference value obtained accordingly. One shall appreciate that such weighing factors may depend on the methodology used to quantify biomarkers and measure or score parameters, and for each particular experimental setting may be determined and comprised in a model suitable for diagnosis, prediction and/or prognosis of the diseases and conditions as taught herein. Various methods can be used for the purpose of establishing such models, e.g., support vector machine, Bayes classifiers, logistic regression, etc. (Cruz et al. Applications of Machine Learning in Cancer Prediction and Prognosis. Cancer Informatics 2007; 2; 59-77).

In a particular embodiment of the methods of the invention, the first step comprises determining the phosphorylation of transferrin, more particularly the phosphorylation of the peptides listed in Table 1. In an alternate embodiment, the first step comprises determining the phosphorylation of one or more sequences listed in SEQ ID Nos: 1-18, more particularly the phosphorylation of one or more serine, tyrosine or threonine of the peptides listed in Table 1. In yet another embodiment, the first step comprises determining the post-transitional modification (preferably phosphorylation) of one or more amino acid residues of transferrin selected from the group consisting of: K359; K37; K508; K546; S136; S144; S298; S305; S306; S389; S468; S520; S63; S688; T139; T340; T349; T355; T36; T476; T537; T654; T686; T694; Y155; Y207; Y257; Y533; Y534; Y536; Y593; Y64; Y666; Y669; and Y674 of transferrin protein, particularly human transferrin protein, or in a phosphorylatable, positionally equivalent amino acid residue of another transferrin protein as defined by multiple amino acid sequence alignment or in a functionally equivalent variant.

As it is used herein, the term “positionally equivalent” refers to the position of an amino acid of an transferrin protein which, by means of multiple amino acid sequence alignment of transferrin protein, corresponds to K359; K37; K508; K546; S 136; S 144; S298; S305; S306; S389; S468; S520; S63; S688; T139; T340; T349; T355; T36; T476; T537; T654; T686; T694; Y155; Y207; Y257; Y533; Y534; Y536; Y593; Y64; Y666; Y669; and Y674 of human transferrin protein.

Multiple sequence alignment can be carried out by means of the algorithm implemented in the CLUSTALW2 program (using standard parameters (alignment type: slow; matrix: Gonnet; gap open: 10; gap extension: 0.1; KTUP: 1; Window length: 5; Score type: percent; Top Diags: 5 and Pair Gap: 3). In another embodiment, multiple sequence alignment can be carried out by means of the algorithm implemented in the CL UST AL OMEGA program using standard parameters (HHalign algorithm with default parameters and the default transition matrix is Gonnet, with a 6-bit gap opening penalty and a 1-bit gap extension).

A second step of the methods of the invention comprises comparing the level of phosphorylation obtained in the first step of the methods (described further herein) with a reference value.

As it is used herein, the term “reference value” refers to pre determined criteria used as a reference for evaluating the values or data obtained from the samples collected from a subject. The reference value or reference level can be an absolute value, a relative value, a value having an upper or lower limit, a range of values, an average value, a median value, a mean value, or a value compared to a particular control or baseline value. In some embodiments, the reference value is not necessarily determined every time. A reference value can be based on a value of an individual sample such as, for example, a value obtained from a sample from the subject being analyzed, but at an earlier point in time. This earlier time may be prior to the individual being diagnosed with dementia or may be prior to a therapeutic intervention, for example but by no means limited to prescription of treatment, for example, therapeutic drugs and/or lifestyle changes or the like as discussed herein. In this manner, progression of the dementia and/or effectiveness of the therapeutic intervention can be monitored. The reference value can be based on a large number of samples, such as a population of subjects of the matching chronological age group, or based on a pool of samples including or excluding the sample being analyzed. In a particular embodiment, the reference value for a phosphorylated amino acid residue in transferrin protein is the level of phosphorylation of said residue of the protein in a sample from a subject or population of healthy or control subjects, i.e., those that do not exhibit any neurodegenerative disorder, specifically those that do not exhibit Alzheimer's disease or a cognitive disorder similar to said disease. Typical reference samples will generally be obtained from subjects who are clinically well documented.

Reference values as employed herein may be established according to known procedures previously employed for other test panels comprising biomarkers and/or clinical parameters. Reference values may be established either within (i.e., constituting a step of) or external to (i.e., not constituting a step of) the methods and uses as taught herein. Accordingly, any one of the methods or uses taught herein may comprise a step of establishing a requisite reference value.

In some embodiments of the invention, following a positive result, the individual is subjected to cognitive tests and/or brain imaging to determine if the individual has Alzheimer's disease or another cognitive disorder similar to Alzheimer's such as Parkinson's disease or another form of dementia. As will be appreciated by one of skill in the art, subsequent screening or testing may show that the positive result is in fact a false positive.

In some embodiments of the invention, following a positive result, the individual is scheduled for cognitive tests and/or brain imaging to determine if the individual has Alzheimer's disease.

In some embodiments of the invention, a positive result indicates that the individual has dementia. The dementia may be associated with Alzheimer's disease, Parkinson's disease or another form of dementia.

Accordingly, in a second aspect, the invention relates to an m vitro method for designing a personalized therapy in a subject suffering from mild cognitive impairment (second method of the invention), which method comprises:
a) determining in a sample from the subject the level of phosphorylation of transferrin protein or in a functionally equivalent variant and
b) comparing the level of phosphorylation obtained in a) to a reference value, wherein an increase in the level of phosphorylation of transferrin protein or m a functionally equivalent variant compared to the reference value is indicative that said subject is susceptible to receive a therapy for the prevention and/or treatment of Alzheimer's disease or a cognitive disorder similar to said disease.

In a third aspect, the invention relates to an in vitro method for selecting a patient susceptible to be treated with a therapy for the prevention and/or treatment of Alzheimer's or a cognitive disorder similar to said disease (third method of the invention), which method comprises
a) determining in a sample from the subject the level of phosphorylation of transferrin protein or in a functionally equivalent variant and
b) comparing the level of phosphorylation obtained in a) to a reference value, wherein an increase in the level of phosphorylation of transferrin protein or m a functionally equivalent variant compared to the reference value is indicative that said subject is a candidate for receiving a therapy for the prevention and/or treatment of Alzheimer's disease or a cognitive disorder similar to said disease.

In some embodiments of the invention, following a positive result, the individual is assessed and assigned or adopts a life style change as recommended by one of skill or knowledge in the area of treatment of dementia and/or living with dementia. Alternatively, the individual may be assigned preventative care and/or pre-emptive therapeutic treatment as known in the art and as discussed herein.

In other embodiments, the individual is assigned to or participates in a research study.

In a particular embodiment of the second method of the invention, the subject is a human and the therapy is for the prevention and/or treatment of dementia, or Alzheimer's disease or other cognitive disorder similar said disease.

As it is used herein, the term “preventive therapy” refers to the prevention of or a set of prophylactic measures for preventing a disease to prevent or delay the onset of the symptomatology of the disease. Particularly, said term refers to the prevention of or the set of measures for preventing the onset or delaying the clinical symptomatology associated with dementia, or Alzheimer's disease or a cognitive disorder similar to said disease. Desired clinical results associated with the administration of said treatment to a subject include, but are not limited to, stabilizing the pathological state of the disease, delaying the progression of the disease or improving the physiological state of the subject.

As it is used herein, “therapy for the treatment” refers to the tentative recovery of a health issue, generally after a diagnosis, specifically of Alzheimer's disease or a cognitive disorder similar to said disease. So it is not necessarily a cure, i.e., a complete reversion of a disease. Therefore, as it is used herein “treatment” covers any treatment of a disease, a disorder or a condition of a mammal, particularly a human being, and includes inhibiting the disease or condition, i.e., stopping its development; or alleviating the disease or condition, i.e., causing the regression of the disease or condition or improving one or more symptoms of the disease or condition. The population of subjects treated by means of the method includes a subject suffering from the unwanted condition or disease, as well as subjects at risk of developing the condition or disease. Therefore, a person skilled in the art understands that a treatment can improve the condition of the patient, but it may not be a complete cure for the disease.

Preventive or curative treatments suitable in Alzheimer's disease or in a cognitive disorder similar to said disease include, but are not limited to, choline-esterase inhibitors such as, for example, donepezil hydrochloride (Aricept), rivastigmine (Exelon) and galantamine (Reminyl), N-methyl D-aspartate (NMDA) receptor antagonists (e.g., memantine), or monoclonal antibodies such as solanezumab and bapineuzumab. In some embodiments, the individual is prescribed an acetylcholinesterase inhibitor and/or a NMDA receptor inhibitor on a regimen or schedule, for example, daily or as needed.

As it is used herein, the term “select” refers to the action of choosing a subject to put said subject under a preventive or curative treatment for Alzheimer's disease or a cognitive disorder similar to said disease.

In a particular embodiment of the third method of the invention, the subject is a human and the therapy is for the prevention and/or treatment of Alzheimer's disease.

The first, second and third methods of the invention comprise in a first step determining in a sample from a subject the level of phosphorylation in tyrosine, serine and threonine residues in transferrin protein or in a functionally equivalent variant.

As it is used herein, “sample” refers to the biological material isolated from a subject. The sample can be isolated from any suitable biological fluid or tissue including, by way of illustrative and non-limiting example, cerebrospinal fluid (CSF), blood serum, blood plasma, tears, sweat, saliva, urine and feces.

In a particular embodiment of the methods of the invention, the sample is selected from the group consisting of cerebrospinal fluid, blood serum, blood plasma, blood and peripheral blood mononuclear cells. For example, whole blood can be collected from the patients and serum is prepared therefrom by allowing the blood to clot. In some embodiments, the serum sample may be treated with deglycosylase and/or phosphate and protease inhibitor. As will be appreciated by one of skill in the mi, serum is less invasive and easy to collect. However, if a patient comes for another treatment and CSF is collected then that CSF can be screened.

As it is used herein, the term “subject” refers to a member of a mammalian animal species and includes, but is not limited to, domestic animals, primates and humans. In a particular embodiment, the subject is preferably a male or female human being of any age or race. In another particular embodiment, the subject is a dog. In a particular embodiment, the subject suffers from dementia, such as mild cognitive impairment, Alzheimer's or other similar cognitive disorder.

The “transferrin” “(TF)” of the present embodiment refers to a protein that 1 s substantially identical to a glycoprotein having 679 ammo acids generally referred to as transferrin, and which also includes a genetic polymorph, a genetic variant, or a splicing variant thereof. Transferrin is a plasma protein produced mainly in the liver that works as an iron transport molecule by binding two atoms of iron per molecule thereof, and is known to be involved in in vivo hematopoietic function and iron metabolism. Human “transferrin” protein corresponds with the protein identified in the Uniprot database as P02787 (12 Sep. 2018).

In the context of the present invention, the term “functionally equivalent variant of transferrin protein” includes (i) variants of transferrin protein in which one or more of the amino acid residues are substituted with a conserved or non-conserved amino acid residue (preferably a conserved amino acid residue), wherein such substituted amino acid residue may or may be not be a residue encoded by the genetic code, (ii) variants comprising an insertion or a deletion of one or more amino acids and playing the same function as transferrin protein, as well as (iii) fragments thereof.

The variants according to the invention preferably have a sequence identity with the transferrin amino acid sequence of at least 50%, at least 60%, at least 70%, at least 80%, at least 90%, at least 91%, at least 92%, at least 93%, at least 94, at least 95%, at least 96%, at least 97%, at least 98% or at least 99%. The degree of identity between the variants and the specific sequences of transferrin protein defined above can be determined using algorithms and computational methods that are well-known for those skilled in the art. The identity between two amino acid sequences is preferably determined using the BLASTP algorithm [BLAST Manual, Altschul, S., et. al., NCBI NLM NIH Bethesda, Md. 20894, Altschul, S., et. al., J. Mol. Biol. 215: 403-410 (1990)).

The reference herein to any transferrin biomarker, nucleic acid, protein or polypeptide may also encompass fragments thereof. Hence, the reference herein to measuring (or measuring the quantity of) any one biomarker, nucleic acid, protein or polypeptide may encompass measuring the biomarker, nucleic acid, protein or polypeptide, such as, e.g., measuring the mature and/or the processed soluble/secreted form (e.g. plasma circulating form) thereof and/or measuring one or more fragments thereof.

The term “fragment” of the transferrin protein, polypeptide or peptide generally refers to N-terminally and/or C-terminally deleted or truncated forms of said protein, polypeptide or peptide. In an embodiment, a fragment may be N-terminally and/or C-terminally truncated by between 1 and about 20 amino acids, such as, e.g., by between 1 and about 15 amino acids, or by between 1 and about 10 amino acids, or by between 1 and about 5 amino acids, compared to the corresponding mature, full-length protein or its soluble or plasma circulating form. In an embodiment, the transferrin fragment is an amino acid sequence comprising any ones listed in Table 1, or a fragment of one of said sequences, wherein the fragment is four to twenty amino acid long and includes the phosphorylatable tyrosine, serine and/or threonine. In an alternate embodiment of the present invention, the transferrin fragment comprises one or more amino acid residues of transferrin selected from the group consisting of: K359; K37; K508; K546; 5136; 5144; 5298; S305; S306; 5389; 5468; 5520; S63; 5688; T139; T340; T349; T355; T36; T476; T537; T654; T686; T694; Y155; Y207; Y257; Y533; Y534; Y536; Y593; Y64; Y666; Y669; and Y674.

As described herein, the “residues of interest” refers to the amino acid residues that are phosphorylatable of transferrin protein, including those listed on Table 1, SEQ ID NOs: 1-18, or the residues K359; K37; K508; K546; S136; S144; S298; 5305; 5306; S389; S468; 5520; S63; S688; T139; T340; T349; T355; T36; T476; T537; T654; T686; T694; Y155; Y207; Y257; Y533; Y534; Y536; Y593; Y64; Y666; Y669; and Y674 of human transferrin protein, or positional equivalents thereof.

In another aspect, the invention discloses phosphorylation site-specific binding molecules that specifically bind at a novel tyrosine, serine and/or threonine phosphorylation site of the invention, and that distinguish between the phosphorylated and unphosphorylated forms. In one embodiment, the binding molecule is an antibody or an antigen-binding fragment thereof. The antibody may specifically bind to an amino acid sequence comprising a phosphorylation site identified in Table 1.

As it is used herein, the term “antibody” seeks to include both chimeric or recombinant antibodies and monoclonal antibodies and polyclonal antibodies or proteolytic fragments thereof, such as Fab or F(ab′) 2 fragments, etc. Furthermore, the DNA encoding the variable region of the antibody can be inserted in other antibodies to thereby produce chimeric antibodies. Single-chain antibodies (scFv) can be polypeptides formed by single chains having the characteristic capacity of an antigen-binding antibody and comprising a pair of amino acid sequences homologous or analogous to the variables regions of the light and heavy chains of immunoglobulins (VH-VL or scFv binding). Polypeptides analogous to the variable regions of the light and heavy chains of an antibody can bind, if desired, through a binding polypeptide. Methods for producing antibodies are well-known and described in the state of the art.

In some embodiments, the antibody or antigen-binding fragment thereof specifically binds the phosphorylated site. In other embodiments, the antibody or antigen-binding fragment thereof specially binds the unphosphorylated site. An antibody or antigen-binding fragment thereof specially binds an amino acid sequence comprising a novel tyrosine, serine and/or threonine phosphorylation site in Table 1 when it does not significantly bind any other site in the parent protein and does not significantly bind a protein other than the parent protein. An antibody of the invention is sometimes referred to herein as a “phospho-specific” antibody.

An antibody or antigen-binding fragment thereof specially binds an antigen when the dissociation constant is ≤1 mM, preferably ≤100 nM, and more preferably ≤10 nM.

In particularly preferred embodiments, an antibody or antigen-binding fragment thereof of the invention specially binds an amino acid sequence comprising a novel tyrosine, serine and/or threonine phosphorylation site shown as a lower case “y,” “s,” or “t” (respectively) in a sequence listed in Table 1.

In some embodiments, an antibody or antigen-binding fragment thereof of the invention specifically binds an amino acid sequence comprising any ones listed in Table I, or a fragment of one of said sequences, wherein the fragment is four to twenty amino acid long and includes the phosphorylatable tyrosine, serine and/or threonine.

In certain embodiments, an antibody or antigen-binding fragment thereof of the invention specially binds an amino acid sequence that comprises a peptide produced by proteolysis of the parent protein with a protease wherein said peptide comprises a novel tyrosine, serine and/or threonine phosphorylation site of the invention. In some embodiments, the peptides are produced from trypsin digestion of the parent protein. The parent protein comprising the novel tyrosine, serine and/or threonine phosphorylation site can be from any species, preferably from a mammal including but not limited to non-human primates, rabbits, mice, rats, goats, cows, sheep, and guinea pigs. In some embodiments, the parent protein is a human protein and the antibody binds an epitope comprising the novel tyrosine, serine and/or threonine phosphorylation site shown by a lower case “y,” “s” or “t” in Table 1. Such peptides include any one of SEQ ID NOs: 1-18.

An antibody of the invention can be an intact, four immunoglobulin chain antibody comprising two heavy chains and two light chains. The heavy chain of the antibody can be of any isotype including IgM, IgG, IgE, IgG, IgA or IgD or sub-isotype including IgG1, IgG2, IgG3, IgG4, IgE1, IgE2, etc. The light chain can be a kappa light chain or a lambda light chain.

Also within the invention are antibody molecules with fewer than 4 chains, including single chain antibodies, Camelid antibodies and the like and components of the antibody, including a heavy chain or a light chain. The term “antibody” (or “antibodies”) refers to all types of immunoglobulins. The term “an antigen-binding fragment of an antibody” refers to any portion of an antibody that retains specific binding of the intact antibody. An exemplary antigen-binding fragment of an antibody is the heavy chain and/or light chain CDR, or the heavy and/or light chain variable region. The term “does not bind,” when appeared in context of an antibody's binding to one phospho-form (e.g., phosphorylated form) of a sequence, means that the antibody does not substantially react with the other phospho-form (e.g., nonphosphorylated form) of the same sequence. One of skill in the art will appreciate that the expression may be applicable in those instances when (1) a phospho-specific antibody either does not apparently bind to the non-phospho form of the antigen as ascertained in commonly used experimental detection systems (Western blotting, IHC, Immunofluorescence, etc.); (2) where there is some reactivity with the surrounding amino acid sequence, but that the phosphorylated residue is an immunodominant feature of the reaction. In cases such as these, there is an apparent difference in affinities for the two sequences. Dilutional analyses of such antibodies indicates that the antibodies apparent affinity for the phosphorylated form is at least 10-100 fold higher than for the non-phosphorylated form; or where (3) the phospho-specific antibody reacts no more than an appropriate control antibody would react under identical experimental conditions. A control antibody preparation might be, for instance, purified immunoglobulin from a pre-immune animal of the same species, an isotype- and species-matched monoclonal antibody. Tests using control antibodies to demonstrate specificity are recognized by one of skill in the art as appropriate and definitive.

In some embodiments an immunoglobulin chain may comprise in order from 5′ to 3′, a variable region and a constant region. The variable region may comprise three complementarity determining regions (CDRs), with interspersed framework (FR) regions for a structure FRI, CDR1, FR2, CDR2, FR3, CDR3 and FR4. Also within the invention are heavy or light chain variable regions, framework regions and CDRs. An antibody of the invention may comprise a heavy chain constant region that comprises some or all of a CH 1 region, hinge, CH2 and CH3 region.

An antibody of the invention may have a binding affinity (Ko) of 1×10⁻⁷M or less. In other embodiments, the antibody binds with a K_{D} of 1×10⁻⁸ M, 1×10⁻⁹ M, 1×10⁻¹⁰ M, 1×10⁻¹¹M, 1×10⁻¹²M or less. In certain embodiments, the K_{D} is 1 pM to 500 pM, between 500 pM to 1 μM, between 1 μM to 100 nM, or between 100 mM to 10 nM.

Antibodies of the invention can be derived from any species of animal, preferably a mammal. Non-limiting exemplary natural antibodies include antibodies derived from human, chicken, goats, and rodents (e.g., rats, mice, hamsters and rabbits), including transgenic rodents genetically engineered to produce human antibodies (see, e.g., Lonberg et al., WO93/12227; U.S. Pat. No. 5,545,806; and Kucherlapati, et al., WO91/10741; U.S. Pat. No. 6,150,584, which are herein incorporated by reference in their entirety). Natural antibodies are the antibodies produced by a host animal. “Genetically altered antibodies” refer to antibodies wherein the amino acid sequence has been varied from that of a native antibody. Because of the relevance of recombinant DNA techniques to this application, one need not be confined to the sequences of amino acids found in natural antibodies; antibodies can be redesigned to obtain desired characteristics. The possible variations are many and range from the changing of just one or a few amino acids to the complete redesign of, for example, the variable or constant region. Changes in the constant region will, in general, be made in order to improve or alter characteristics, such as complement fixation, interaction with membranes and other effector functions. Changes in the variable region will be made in order to improve the antigen binding characteristics.

The antibodies of the invention include antibodies of any isotype including IgM, IgG, IgD, IgA and IgE, and any sub-isotype, including IgG1, IgG2a, IgG2b, IgG3 and IgG4, IgE1, IgE2 etc. The light chains of the antibodies can either be kappa light chains or lambda light chains.

Antibodies disclosed in the invention may be polyclonal or monoclonal. As used herein, the term “epitope” refers to the smallest portion of a protein capable of selectively binding to the antigen binding site of an antibody. It is well accepted by those skilled in the art that the minimal size of a protein epitope capable of selectively binding to the antigen binding site of an antibody is about five or six to seven amino acids.

Other antibodies specifically contemplated are oligoclonal antibodies. As used herein, the phrase “oligoclonal antibodies” refers to a pre determined mixture of distinct monoclonal antibodies. See, e.g., PCT publication WO 95/20401; U.S. Pat. Nos. 5,789,208 and 6,335,163. In one embodiment, oligoclonal antibodies consisting of a predetermined mixture of antibodies against one or more epitopes are generated in a single cell. In other embodiments, oligoclonal antibodies comprise a plurality of heavy chains capable of pairing with a common light chain to generate antibodies with multiple specificities (e.g., PCT publication WO 04/009618). Oligoclonal antibodies are particularly useful when it is desired to target multiple epitopes on a single target molecule. In view of the assays and epitopes disclosed herein, those skilled in the art can generate or select antibodies or mixtures of antibodies that are applicable for an intended purpose and desired need.

Recombinant antibodies against the phosphorylation sites identified in the invention are also included in the present application. These recombinant antibodies have the same amino acid sequence as the natural antibodies or have altered amino acid sequences of the natural antibodies in the present application. They can be made in any expression systems including both prokaryotic and eukaryotic expression systems or using phage display methods (see, e.g., Dower et al., WO91/17271 and McCafferty et al., WO92/01047; U.S. Pat. No. 5,969,108, which are herein incorporated by reference in their entirety).

Antibodies can be engineered in numerous ways. They can be made as single-chain antibodies (including small modular immunopharmaceuticals or SMIPs™), Fab and F(ab′)2 fragments, etc. Antibodies can be humanized, chimerized, deimmunized, or fully human. Numerous publications set forth the many types of antibodies and the methods of engineering such antibodies. For example, see U.S. Pat. Nos. 6,355,245; 6,180,370; 5,693,762; 6,407,213; 6,548,640; 5,565,332; 5,225,539; 6,103,889; and 5,260,203.

The genetically altered antibodies should be functionally equivalent to the abovementioned natural antibodies. In certain embodiments, modified antibodies provide improved stability or/and therapeutic efficacy. Examples of modified antibodies include those with conservative substitutions of amino acid residues, and one or more deletions or additions of amino acids that do not significantly deleteriously alter the antigen binding utility. Substitutions can range from changing or modifying one or more amino acid residues to complete redesign of a region as long as the therapeutic utility is maintained. Antibodies of this application can be modified post-translationally (e.g., acetylation, and/or phosphorylation) or can be modified synthetically (e.g., the attachment of a labeling group).

Antibodies with engineered or variant constant or Fe regions can be useful in modulating effector functions, such as, for example, antigen-dependent cytotoxicity (ADCC) and complement-dependent cytotoxicity (CDC). Such antibodies with engineered or variant constant or Fe regions may be useful in instances where a parent singling protein (Table 1) is expressed in normal tissue; variant antibodies without effector function in these instances may elicit the desired therapeutic response while not damaging normal tissue. Accordingly, certain aspects and methods of the present disclosure relate to antibodies with altered effector functions that comprise one or more amino acid substitutions, insertions, and/or deletions.

In certain embodiments, genetically altered antibodies are chimeric antibodies and humanized antibodies.

The chimeric antibody is an antibody having portions derived from different antibodies. For example, a chimeric antibody may have a variable region and a constant region derived from two different antibodies. The donor antibodies may be from different species. In certain embodiments, the variable region of a chimeric antibody is non-human, e.g., murine, and the constant region is human.

The genetically altered antibodies used in the invention include CDR grafted humanized antibodies. In one embodiment, the humanized antibody comprises heavy and/or light chain CDRs of a non-human donor immunoglobulin and heavy chain and light chain frameworks and constant regions of a human acceptor immunoglobulin. The method of making humanized antibody is disclosed in U.S. Pat. Nos. 5,530,101; 5,585,089; 5,693,761; 5,693,762; and 6,180,370 each of which is incorporated herein by reference in its entirety.

Antigen-binding fragments of the antibodies of the invention, which retain the binding specificity of the intact antibody, are also included in the invention. Examples of these antigen-binding fragments include, but are not limited to, partial or full heavy chains or light chains, variable regions, or CDR regions of any phosphorylation site-specific antibodies described herein.

In one embodiment of the application, the antibody fragments are truncated chains (truncated at the carboxyl end). In certain embodiments, these truncated chains possess one or more immunoglobulin activities (e.g., complement fixation activity). Examples of truncated chains include, but are not limited to, Fab fragments (consisting of the VL, VH, CL and CH1 domains); F d fragments (consisting of the VH and CH1 domains); Fv fragments ( consisting of VL and VH domains of a single chain of an antibody); dAb fragments (consisting of a VH domain); isolated CDR regions; (Fab′) 2 fragments, bivalent fragments (comprising two Fab fragments linked by a disulphide bridge at the hinge region). The truncated chains can be produced by conventional biochemical techniques, such as enzyme cleavage, or recombinant DNA techniques, each of which is known in the art. These polypeptide fragments may be produced by proteolytic cleavage of intact antibodies by methods well known in the art, or by inserting stop codons at the desired locations in the vectors using site-directed mutagenesis, such as after CHI to produce Fab fragments or after the hinge region to produce (Fab′) 2 fragments. Single chain antibodies may be produced by joining VL- and VH-coding regions with a DNA that encodes a peptide linker connecting the VL and VH protein fragments

Papain digestion of antibodies produces two identical antigen-binding fragments, called “Fab” fragments, each with a single antigen-binding site, and a residual “Fe” fragment, whose name reflects its ability to crystallize readily. Pepsin treatment of an antibody yields an F(ab′) 2 fragment that has two antigen-combining sites and is still capable of cross-linking antigen.

“Fv” usually refers to the minimum antibody fragment that contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the CD Rs confer antigen-binding specificity to the antibody. However, even a single variable domain (or half of an Fv comprising three CD Rs specific for an antigen) has the ability to recognize and bind antigen, although likely at a lower affinity than the entire binding site.

Thus, in certain embodiments, the antibodies of the application may comprise 1, 2, 3, 4, 5, 6, or more CDRs that recognize the phosphorylation sites identified in Table 1.

The Fab fragment also contains the constant domain of the light chain and the first constant domain (CHI) of the heavy chain. Fab′ fragments differ from Fab fragments by the addition of a few residues at the carboxy terminus of the heavy chain CH 1 domain including one or more cysteines from the antibody hinge region. Fab′-SH is the designation herein for Fab′ in which the cysteine residue(s) of the constant domains bear a free thiol group. F(ab′) 2 antibody fragments originally were produced as pairs of Fab′ fragments that have hinge cysteines between them. Other chemical couplings of antibody fragments are also known.

“Single-chain Fv” or “scFv” antibody fragments comprise the VH and VL domains of an antibody, wherein these domains are present in a single polypeptide chain. In certain embodiments, the Fv polypeptide further comprises a polypeptide linker between the VH and V_{L} domains that enables the scFv to form the desired structure for antigen binding. For a review of scFv see Pluckthun in The Pharmacology of Monoclonal Antibodies, vol. 113, Rosenburg and Moore, eds. (Springer-Verlag: New York, 1994), pp. 269-315.

SMIPs are a class of single-chain peptides engineered to include a target binding region and effector domain (CH2 and CH3 domains). See, e.g., U.S. Patent Application Publication No. 20050238646. The target binding region may be derived from the variable region or CDRs of an antibody, e.g., a phosphorylation site-specific antibody of the application. Alternatively, the target binding region is derived from a protein that binds a phosphorylation site.

Bispecific antibodies may be monoclonal, human or humanized antibodies that have binding specificities for at least two different antigens. In the present case, one of the binding specificities is for the phosphorylation site, the other one is for any other antigen, such as for example, a cell-surface protein or receptor or receptor subunit. Alternatively, a therapeutic agent may be placed on one arm. The therapeutic agent can be a drug, toxin, enzyme, DNA, radionuclide, etc.

In some embodiments, the antigen-binding fragment can be a diabody. The term “diabody” refers to small antibody fragments with two antigen-binding sites, which fragments comprise a heavy-chain variable domain (V_{H}) connected to a light-chain variable domain (V_{L}) in the same polypeptide chain (V_{H}—V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90: 6444-6448 (1993).

Camelid antibodies refer to a unique type of antibodies that are devoid of light chain, initially discovered from animals of the camelid family. The heavy chains of these so-called heavy-chain antibodies bind their antigen by one single domain, the variable domain of the heavy immunoglobulin chain, referred to as VHH. VHHs show homology with the variable domain of heavy chains of the human VHIII family. The VHHs obtained from an immunized camel, dromedary, or llama have a number of advantages, such as effective production in microorganisms such as Saccharomyces cerevisiae.

In certain embodiments, single chain antibodies, and chimeric, humanized or primatized (CDR-grafted) antibodies, as well as chimeric or CDR-grafted single chain antibodies, comprising portions derived from different species, are also encompassed by the present disclosure as antigen-binding fragments of an antibody. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., U.S. Pat. Nos. 4,816,567 and 6,331,415; 4,816,397; European Patent No. 0,120,694; WO 86/01533; European Patent No. 0,194,276 B1; U.S. Pat. No. 5,225,539; and European Patent No. 0,239,400 B1. See also, Newman et al., BioTechnology, 10: 1455-1460 (1992), regarding primatized antibody. See, e.g., Ladner et al., U.S. Pat. No. 4,946,778; and Bird et al., Science, 242: 423-426 (1988)), regarding single chain antibodies.

In addition, functional fragments of antibodies, including fragments of chimeric, humanized, primatized or single chain antibodies, can also be produced. Functional fragments of the subject antibodies retain at least one binding function and/or modulation function of the full-length antibody from which they are derived.

Since the immunoglobulin-related genes contain separate functional regions, each having one or more distinct biological activities, the genes of the antibody fragments may be fused to functional regions from other genes (e.g., enzymes, U.S. Pat. No. 5,004,692, which is incorporated by reference in its entirety) to produce fusion proteins or conjugates having novel properties.

Non-immunoglobulin binding polypeptides are also contemplated. For example, CDRs from an antibody disclosed herein may be inserted into a suitable non-immunoglobulin scaffold to create a non-immunoglobulin binding polypeptide. Suitable candidate scaffold structures may be derived from, for example, members of fibronectin type III and cadherin superfamilies.

Also contemplated are other equivalent non-antibody molecules, such as protein binding domains or aptamers, which bind, in a phospho-specific manner, to an amino acid sequence comprising a novel phosphorylation site of the invention. See, e.g., Neuberger et al. (Neuberger et al., 1984). Aptamers are oligonucleic acid or peptide molecules that bind a specific target molecule. DNA or RNA aptamers are typically short oligonucleotides, engineered through repeated rounds of selection to bind to a molecular target. Peptide aptamers typically consist of a variable peptide loop attached at both ends to a protein scaffold. This double structural constraint generally increases the binding affinity of the peptide aptamer to levels comparable to an antibody (nanomolar range).

The phosphorylation site-specific antibodies disclosed in the invention may be used singly or in combination. The antibodies may also be used in an array format for high throughput uses. An antibody microarray is a collection of immobilized antibodies, typically spotted and fixed on a solid surface (such as glass, plastic and silicon chip).

In certain embodiments, the phosphorylation site specific antibodies disclosed in the invention are especially indicated for diagnostic and therapeutic applications as described herein. Accordingly, the antibodies may be used in therapies, including combination therapies, in the diagnosis and prognosis of disease, as well as in the monitoring of disease progression. The invention, thus, further includes compositions comprising one or more embodiments of an antibody or an antigen binding portion of the invention as described herein. The composition may further comprise a pharmaceutically acceptable carrier. The composition may comprise two or more antibodies or antigen-binding portions, each with specificity for a different novel tyrosine, serine and/or threonine phosphorylation site of the invention or two or more different antibodies or antigen-binding portions all of which are specific for the same novel tyrosine, serine and/or threonine phosphorylation site of the invention. A composition of the invention may comprise one or more antibodies or antigen-binding portions of the invention and one or more additional reagents, diagnostic agents or therapeutic agents.

The present application provides for the polynucleotide molecules encoding the antibodies and antibody fragments and their analogs described herein. Because of the degeneracy of the genetic code, a variety of nucleic acid sequences encode each antibody amino acid sequence. The desired nucleic acid sequences can be produced by de novo solid-phase DNA synthesis or by PCR mutagenesis of an earlier prepared variant of the desired polynucleotide. In one embodiment, the codons that are used comprise those that are typical for human or mouse (see, e.g., Nakamura, Y., Nucleic Acids Res. 28: 292 (2000)).

The invention also provides immortalized cell lines that produce an antibody of the invention. For example, hybridoma clones, constructed as described above, that produce monoclonal antibodies to the targeted signaling protein phosphorylation sties disclosed herein are also provided. Similarly, the invention includes recombinant cells producing an antibody of the invention, which cells may be constructed by well known techniques; for example the antigen combining site of the monoclonal antibody can be cloned by PCR and single-chain antibodies produced as phage-displayed recombinant antibodies or soluble antibodies in E. coli (see, e.g., ANTIBODY ENGINEERING PROTOCOLS, 1995, Humana Press, Sudhir Paul editor.)

In another aspect, the invention provides a method for making phosphorylation site-specific antibodies.

Polyclonal antibodies of the invention may be produced according to standard techniques by immunizing a suitable animal (e.g., rabbit, goat, etc.) with an antigen comprising a novel tyrosine, serine and/or threonine phosphorylation site of the invention. (i.e. a phosphorylation site shown in Table 1) in either the phosphorylated or unphosphorylated state, depending upon the desired specificity of the antibody, collecting immune serum from the animal, and separating the polyclonal antibodies from the immune serum, in accordance with known procedures and screening and isolating a polyclonal antibody specific for the novel tyrosine, serine and/or threonine phosphorylation site of interest as further described below. Methods for immunizing non-human animals such as mice, rats, sheep, goats, pigs, cattle and horses are well known in the art. See, e.g., Harlow and Lane, Antibodies: A Laboratory Manual, New York: Cold Spring Harbor Press, 1990.

The immunogen may be the full length protein or a peptide comprising the novel tyrosine, serine and/or threonine phosphorylation site of interest. In some embodiments the immunogen is a peptide of from 4 to 20 amino acids in length, alternatively about 8 to 17 amino acids in length. In some embodiments, the peptide antigen desirably will comprise about 3 to 8 amino acids on each side of the phosphorylatable tyrosine, serine and/or threonine. In yet other embodiments, the peptide antigen desirably will comprise four or more amino acids flanking each side of the phosphorylatable amino acid and encompassing it. Peptide antigens suitable for producing antibodies of the invention may be designed, constructed and employed in accordance with well-known techniques. See, e.g., Antibodies: A Laboratory Manual, Chapter 5, p. 75-76, Harlow & Lane Eds., Cold Spring Harbor Laboratory (1988); Czernik, Methods In Enzymology, 201: 264-283 (1991); Merrifield, J Am. Chem. Soc. 85: 21-49 (1962)).

Suitable peptide antigens may comprise all or partial sequence of a fragment as set forth in Table 1.

Particularly preferred immunogens are peptides comprising any one of the novel tyrosine, serine and/or threonine phosphorylation site shown as a lower case “y,” “s” or “t” the sequences listed in Table 1 selected from the group consisting of SEQ ID NOS: 1-18. In one embodiment of the present invention, the peptides of the present invention are fragments comprising one or more amino acid residues of transferrin selected from the group consisting of: K359; K37; K508; K546; 5136; 5144; 5298; 5305; 5306; 5389; 5468; 5520; S63; 5688; T139; T340; T349; T355; T36; T476; T537; T654; T686; T694; Y155; Y207; Y257; Y533; Y534; Y536; Y593; Y64; Y666; Y669; and Y674.

In some embodiments the immunogen is administered with an adjuvant. Suitable adjuvants will be well known to those of skill in the art. Exemplary adjuvants include complete or incomplete Freund's adjuvant, RIBI (muramyl dipeptides) or ISCOM (immunostimulating complexes).

When the above-described methods are used for producing polyclonal antibodies, following immunization, the polyclonal antibodies which secreted into the bloodstream can be recovered using known techniques. Purified forms of these antibodies can, of course, be readily prepared by standard purification techniques, such as for example, affinity chromatography with Protein A, anti-immunoglobulin, or the antigen itself. In any case, in order to monitor the success of immunization, the antibody levels with respect to the antigen in serum will be monitored using standard techniques such as ELISA, RIA and the like.

Monoclonal antibodies of the invention may be produced by any of a number of means that are well-known in the art. In some embodiments, antibody-producing B cells are isolated from an animal immunized with a peptide antigen as described above. The B cells may be from the spleen, lymph nodes or peripheral blood. Individual B cells are isolated and screened as described below to identify cells producing an antibody specific for the novel tyrosine, serine and/or threonine phosphorylation site of interest. Identified cells are then cultured to produce a monoclonal antibody of the invention.

Alternatively, a monoclonal phosphorylation site-specific antibody of the invention may be produced using known hybridoma technology, in a hybridoma cell line according to the well-known technique of Kohler and Milstein. See Nature 265: 495-97 (1975); Kohler and Milstein, Eur. J Immunol. 6: 511 (1976); see also, Current Protocols in Molecular Biology, Ausubel et al. Eds. (1989). Monoclonal antibodies so produced are highly specific, and improve the selectivity and specificity of diagnostic assay methods provided by the invention. For example, a solution containing the appropriate antigen may be injected into a mouse or other species and, after a sufficient time (in keeping with conventional techniques), the animal is sacrificed and spleen cells obtained. The spleen cells are then immortalized by any of a number of standard means. Methods of immortalizing cells include, but are not limited to, transfecting them with oncogenes, infecting them with an oncogenic virus and cultivating them under conditions that select for immortalized cells, subjecting them to carcinogenic or mutating compounds, fusing them with an immortalized cell. See, e.g., Harlow and Lane, supra. Typically the antibody producing cell and the immortalized cell with which it is fused are from the same species. Rabbit fusion hybridomas, for example, may be produced as described in U.S. Pat. No. 5,675,063, C. Knight, Issued Oct. 7, 1997. The immortalized antibody producing cells, such as hybridoma cells, are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

The invention also encompasses antibody-producing cells and cell lines, such as hybridomas, as described above.

Polyclonal or monoclonal antibodies may also be obtained through in vitro immunization. For example, phage display techniques can be used to provide libraries containing a repertoire of antibodies with varying affinities for a particular antigen. Techniques for the identification of high affinity human antibodies from such libraries are described by Griffiths et al., (1994) EMBO J., 13:3245-3260; Nissim et al., ibid, pp. 692-698 and by Griffiths et al., ibid, 12:725-734, which are incorporated by reference.

The antibodies may be produced recombinantly using methods known in the art for example, according to the methods disclosed in U.S. Pat. No. 4,349,893 (Reading) or U.S. Pat. No. 4,816,567 (Cabilly et al.) The antibodies may also be chemically constructed by specific antibodies made according to the method disclosed in U.S. Pat. No. 4,676,980 (Segel et al.)

Once a desired phosphorylation site-specific antibody is identified, polynucleotides encoding the antibody, such as heavy, light chains or both (or single chains in the case of a single chain antibody) or portions thereof such as those encoding the variable region, may be cloned and isolated from antibody-producing cells using means that are known in the art. For example, the antigen combining site of the monoclonal antibody can be cloned by PCR and single-chain antibodies produced as phage-displayed recombinant antibodies or soluble antibodies in E. coli (see, e.g., Antibody Engineering Protocols, 1995, Humana Press, Sudhir Paul editor.)

Accordingly, in a further aspect, the invention provides such nucleic acids encoding the heavy chain, the light chain, a variable region, a framework region or a CDR of an antibody of the invention. In some embodiments, the nucleic acids are operably linked to expression control sequences. The invention, thus, also provides vectors and expression control sequences useful for the recombinant expression of an antibody or antigen-binding portion thereof of the invention. Those of skill in the art will be able to choose vectors and expression systems that are suitable for the host cell in which the antibody or antigen-binding portion is to be expressed.

Monoclonal antibodies of the invention may be produced recombinantly by expressing the encoding nucleic acids in a suitable host cell under suitable conditions. Accordingly, the invention further provides host cells comprising the nucleic acids and vectors described above.

Monoclonal Fab fragments may also be produced in Escherichia coli by recombinant techniques known to those skilled in the art. See, e.g., W. Huse, Science 246: 1275-81 (1989); Mullinax et al., Proc. Nat'l Acad. Sci. 87: 8095 (1990).

If monoclonal antibodies of a single desired isotype are preferred for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Acad. Sci., 82: 8653 (1985); Spira et al., J. Immunol. Methods, 74: 307 (1984)). Alternatively, the isotype of a monoclonal antibody with desirable propertied can be changed using antibody engineering techniques that are well-known in the art.

Phosphorylation site-specific antibodies of the invention, whether polyclonal or monoclonal, may be screened for epitope and phospho-specificity according to known techniques. See, e.g., Czernik et al., Methods in Enzymology, 201: 264-283 (1991). For example, the antibodies may be screened against the phosphorylated and/or unphosphorylated peptide library by ELISA to ensure specificity for both the desired antigen (i.e. that epitope including a phosphorylation site of the invention and for reactivity only with the phosphorylated (or unphosphorylated) form of the antigen. Peptide competition assays may be carried out to confirm lack of reactivity with other phospho-epitopes on the parent protein. The antibodies may also be tested by Western blotting against cell preparations containing the parent signaling protein, e.g., cell lines over-expressing the parent protein, to confirm reactivity with the desired phosphorylated epitope/target.

Specificity against the desired phosphorylated epitope may also be examined by constructing mutants lacking phosphorylatable residues at positions outside the desired epitope that are known to be phosphorylated, or by mutating the desired phospho-epitope and confirming lack of reactivity. Phosphorylation site-specific antibodies of the invention may exhibit some limited cross-reactivity to related epitopes in non-target proteins. This is not unexpected as most antibodies exhibit some degree of cross-reactivity, and anti-peptide antibodies will often cross-react with epitopes having high homology to the immunizing peptide. See, e.g., Czernik, supra. Cross-reactivity with non-target proteins is readily characterized by Western blotting alongside markers of known molecular weight. Amino acid sequences of cross-reacting proteins may be examined to identify phosphorylation sites with flanking sequences that are highly homologous to that of a phosphorylation site of the invention.

In certain cases, polyclonal antisera may exhibit some undesirable general cross-reactivity to phosphotyrosine, serine and/or threonine itself, which may be removed by further purification of antisera, e.g., over a phosphotyramine column. Antibodies of the invention specifically bind their target protein (i.e. a protein listed in Table 1) only when phosphorylated (or only when not phosphorylated, as the case may be), and do not (substantially) bind to the other form (as compared to the form for which the antibody is specific).

Antibodies may be further characterized via immunohistochemical (IHC) staining using normal and diseased tissues to examine phosphorylation and activation state and level of a phosphorylation site in diseased tissue. IHC may be carried out according to well-known techniques. See, e.g., Antibodies: A Laboratory Manual, Chapter 10, Harlow & Lane Eds., Cold Spring Harbor Laboratory (1988).

Antibodies may be further characterized by flow cytometry carried out according to standard methods. See Chow et al., Cytometry (Communications in Clinical Cytometry) 46: 72-78 (2001). Briefly and by way of example, the following protocol for cytometric analysis may be employed: samples may be centrifuged on Ficoll gradients to remove lysed erythrocytes and cell debris. Adhering cells may be scrapped off plates and washed with PBS. Cells may then be fixed with 2% paraformaldehyde for 10 minutes at 37° C. followed by permeabilization in 90% methanol for 30 minutes on ice. Cells may then be stained with the primary phosphorylation site-specific antibody of the invention (which detects a parent signaling protein enumerated in Table 1), washed and labeled with a fluorescent-labeled secondary antibody. Additional fluorochrome-conjugated marker antibodies (e.g., CD45, CD34) may also be added at this time to aid in the subsequent identification of specific hematopoietic cell types. The cells would then be analyzed on a flow cytometer (e.g. a Beckman Coulter FC500) according to the specific protocols of the instrument used.

Antibodies of the invention may also be advantageously conjugated to fluorescent dyes (e.g. Alexa488, PE) for use in multi-parametric analyses along with other signal transduction (phospho-CrkL, phospho-Erk 1/2) and/or cell marker (CD34) antibodies.

Phosphorylation site-specific antibodies of the invention may specifically bind to transferrin protein or polypeptide listed in Table 1 only when phosphorylated at the specified tyrosine, serine and/or threonine residue, but are not limited only to binding to the listed proteins of human species, per se. The invention includes antibodies that also bind conserved and highly homologous or identical phosphorylation sites in respective signaling proteins from other species (e.g., mouse, rat, monkey, yeast), in addition to binding the phosphorylation site of the human homologue. The term “homologous” refers to two or more sequences or subsequences that have at least about 85%, at least 90%, at least 95%, or higher nucleotide or amino acid residue identity, when compared and aligned for maximum correspondence, as measured using sequence comparison method (e.g., BLAST) and/or by visual inspection. Highly homologous or identical sites conserved in other species can readily be identified by standard sequence comparisons (such as BLAST).

Methods for making bispecific antibodies are within the purview of those skilled in the art. Traditionally, the recombinant production of bispecific antibodies is based on the coexpression of two immunoglobulin heavy-chain/light-chain pairs, where the two heavy chains have different specificities (Milstein and Cuello, Nature, 305:537-539 (1983)). Antibody variable domains with the desired binding specificities (antibody-antigen combining sites) can be fused to immunoglobulin constant domain sequences. In certain embodiments, the fusion is with an immunoglobulin heavy-chain constant domain, including at least part of the hinge, CH2, and CH3 regions. DNAs encoding the immunoglobulin heavy-chain fusions and, if desired, the immunoglobulin light chain, are inserted into separate expression vectors, and are co-transfected into a suitable host organism. For further details of illustrative currently known methods for generating bispecific antibodies see, for example, Suresh et al., Methods in Enzymology, 121:210 (1986); WO 96/27011; Brennan et al., Science 229:81 (1985); Shalaby et al., J. Exp. Med. 175:217-225 (1992); Kostelny et al., J. Immunol. 148(5): 1547-1553 (1992); Hollinger et al., Proc. Natl. Acad. Sci. USA 90:6444-6448 (1993); Gruber et al., J. Immunol. 152:5368 (1994); and Tutt et al., J. Immunol. 147:60 (1991). Bispecific antibodies also include cross-linked or heteroconjugate antibodies. Heteroconjugate antibodies may be made using any convenient cross-linking methods. Suitable cross-linking agents are well known in the art, and are disclosed in U.S. Pat. No. 4,676,980, along with a number of crosslinking techniques.

Various techniques for making and isolating bispecific antibody fragments directly from recombinant cell culture have also been described. For example, bispecific antibodies have been produced using leucine zippers. Kostelny et al., J. Immunol., 148(5): 1547-1553 (1992). The leucine zipper peptides from the Fos and Jun proteins may be linked to the Fab′ portions of two different antibodies by gene fusion. The antibody homodimers may be reduced at the hinge region to form monomers and then re-oxidized to form the antibody heterodimers. This method can also be utilized for the production of antibody homodimers. A strategy for making bispecific antibody fragments by the use of single-chain Fv (scFv) dimers has also been reported. See Gruber et al., J. Immunol., 152:5368 (1994). Alternatively, the antibodies can be “linear antibodies” as described in Zapata et al. Protein Eng. 8(10):1057-1062 (1995). Briefly, these antibodies comprise a pair of tandem Fd segments (V_{H}-C_{H}1-V_{H}-C_{H}1) which form a pair of antigen binding regions. Linear antibodies can be bispecific or monospecific.

To produce the chimeric antibodies, the portions derived from two different species (e.g., human constant region and murine variable or binding region) can be joined together chemically by conventional techniques or can be prepared as single contiguous proteins using genetic engineering techniques. The DNA molecules encoding the proteins of both the light chain and heavy chain portions of the chimeric antibody can be expressed as contiguous proteins. The method of making chimeric antibodies is disclosed in U.S. Pat. Nos. 5,677,427; 6,120,767; and 6,329,508, each of which is incorporated by reference in its entirety.

Fully human antibodies may be produced by a variety of techniques. One example is trioma methodology. The basic approach and an exemplary cell fusion partner, SPAZ-4, for use in this approach have been described by Oestberg et al., Hybridoma 2:361-367 (1983); Oestberg, U.S. Pat. No. 4,634,664; and Engleman et al., U.S. Pat. No. 4,634,666 (each of which is incorporated by reference in its entirety).

Human antibodies can also be produced from non-human transgenic animals having transgenes encoding at least a segment of the human immunoglobulin locus. The production and properties of animals having these properties are described in detail by, see, e.g., Lonberg et al., WO93/12227; U.S. Pat. No. 5,545,806; and Kucherlapati, et al., WO91/10741; U.S. Pat. No. 6,150,584, which are herein incorporated by reference in their entirety.

Various recombinant antibody library technologies may also be utilized to produce fully human antibodies. For example, one approach is to screen a DNA library from human B cells according to the general protocol outlined by Huse et al., Science 246:1275-1281 (1989). The protocol described by Huse is rendered more efficient in combination with phage-display technology. See, e.g., Dower et al., WO 91/17271 and McCafferty et al., WO 92/01047; U.S. Pat. No. 5,969,108, (each of which is incorporated by reference in its entirety).

Eukaryotic ribosome can also be used as means to display a library of antibodies and isolate the binding human antibodies by screening against the target antigen, as described in Coia G, et al., J. Immunol. Methods 1: 254 (1-2):191-7 (2001); Hanes J. et al., Nat. Biotechnol. 18(12): 1287-92 (2000); Proc. Natl. Acad. Sci. U.S.A. 95(24): 14130-5 (1998); Proc. Natl. Acad. Sci. U.S.A. 94(10):4937-42 (1997), each which is incorporated by reference in its entirety.

The yeast system is also suitable for screening mammalian cell-surface or secreted proteins, such as antibodies. Antibody libraries may be displayed on the surface of yeast cells for the purpose of obtaining the human antibodies against a target antigen. This approach is described by Yeung, et al., Biotechnol. Prog. 18(2):212-20 (2002); Boeder, E. T., et al., Nat. Biotechnol. 15(6):553-7 (1997), each of which is herein incorporated by reference in its entirety. Alternatively, human antibody libraries may be expressed intracellularly and screened via the yeast two-hybrid system (W00200729A2, which is incorporated by reference in its entirety).

Recombinant DNA techniques can be used to produce the recombinant phosphorylation site-specific antibodies described herein, as well as the chimeric or humanized phosphorylation site-specific antibodies, or any other genetically-altered antibodies and the fragments or conjugate thereof in any expression systems including both prokaryotic and eukaryotic expression systems, such as bacteria, yeast, insect cells, plant cells, mammalian cells (for example, NSO cells).

Once produced, the whole antibodies, their dimers, individual light and heavy chains, or other immunoglobulin forms of the present application can be purified according to standard procedures of the art, including ammonium sulfate precipitation, affinity columns, column chromatography, gel electrophoresis and the like (see, generally, Scopes, R., Protein Purification (Springer-Verlag, N.Y., 1982)). Once purified, partially or to homogeneity as desired, the polypeptides may then be used therapeutically (including extracorporeally) or in developing and performing assay procedures, immunofluorescent staining, and the like. (See, generally, Immunological Methods, Vols. I and II (Lefkovits and Pernis, eds., Academic Press, N Y, 1979 and 1981).

The degree of phosphorylation of a protein can be determined using any conventional method known by those skilled in the art. Various assays are known for determining the state of phosphorylation of a protein, or the amino acid residue which is phosphorylated in a specific protein, such as, for example, in vitro kinase activity assays using radioactively labeled ATP; two-dimensional electrophoresis of proteins thus phosphorylated and labeled (which allows analyzing how many amino acid residues are phosphorylated in a protein); mass spectrometry of the previously purified protein the state of phosphorylation of which is to be measured; directed mutagenesis followed by the in vitro kinase activity assay with the purified proteins; phosphopeptide analysis which involves separating a phosphorylated protein into two dimensions after trypsin digestion, or the less technically complicated Western blot, which contemplates using antibodies against said protein specifically recognizing the amino acid residue or epitope of the protein which is phosphorylated. Techniques for detecting phosphorylated residues in proteins are well-known by the person skilled in the art and are described in the state of the art. Alternatively, transferrin protein can be immunoprecipitated and the total level of phosphorylation in the residues of interest can be determined by means of Western Blot.

In a further aspect, the invention provides methods for detecting and quantitating phosphorylation at a novel tyrosine, serine and/or threonine phosphorylation site of the invention. For example, peptides, including peptides of the invention, and antibodies of the invention are useful in diagnostic and prognostic evaluation of carcinomas, wherein the disease is associated with the phosphorylation state of a novel phosphorylation site in Table 1, whether phosphorylated or dephosphorylated.

Methods of diagnosis can be performed in vitro using a biological sample from a subject. The phosphorylation state or level at the tyrosine, serine and/or threonine residue identified in Table 1 may be assessed. A change in the phosphorylation state or level at the phosphorylation site, as compared to a control, indicates that the subject is suffering from, or susceptible to, dementia.

In another embodiment, the phosphorylation state or level at a phosphorylation site is determined by an antibody or antigen-binding fragment thereof, wherein the antibody specifically binds the phosphorylation site. The antibody may be one that only binds to the phosphorylation site when the tyrosine, serine and/or threonine residue is phosphorylated, but does not bind to the same sequence when the tyrosine, serine and/or threonine is not phosphorylated; or vice versa.

In particular embodiments, the antibodies of the present application are attached to labeling moieties, such as a detectable marker. One or more detectable labels can be attached to the antibodies. Exemplary labeling moieties include radiopaque dyes, radiocontrast agents, fluorescent molecules, spin-labeled molecules, enzymes, or other labeling moieties of diagnostic value, particularly in radiologic or magnetic resonance imaging techniques.

A radiolabeled antibody in accordance with this disclosure can be used for in vitro diagnostic tests. The specific activity of an antibody, binding portion thereof, probe, or ligand, depends upon the half-life, the isotopic purity of the radioactive label, and how the label is incorporated into the biological agent. In immunoassay tests, the higher the specific activity, in general, the better the sensitivity. Radioisotopes useful as labels, e.g., for use in diagnostics, include iodine (¹³¹I or ¹²⁵I), indium (¹¹¹In) technetium (⁹⁹Tc), phosphorus (³²P), carbon (¹⁴C), and tritium (³H), or one of the therapeutic isotopes listed above.

Fluorophore and chromophore labeled biological agents can be prepared from standard moieties known in the art. Since antibodies and other proteins absorb light having wavelengths up to about 310 nm, the fluorescent moieties may be selected to have substantial absorption at wavelengths above 310 nm, such as for example, above 400 nm. A variety of suitable fluorescers and chromophores are described by Stryer, Science, 162:526 (1968) and Brand et al., Annual Review of Biochemistry, 41:843-868 (1972), which are hereby incorporated by reference. The antibodies can be labeled with fluorescent chromophore groups by conventional procedures such as those disclosed in U.S. Pat. Nos. 3,940,475, 4,289,747, and 4,376,110, which are hereby incorporated by reference.

The control may be parallel samples providing a basis for comparison, for example, biological samples drawn from a healthy subject, or biological samples drawn from healthy tissues of the same subject. Alternatively, the control may be a pre-determined reference or threshold amount. If the subject is being treated with a therapeutic agent, and the progress of the treatment is monitored by detecting the tyrosine, serine and/or threonine phosphorylation state level at a phosphorylation site of the invention, a control may be derived from biological samples drawn from the subject prior to, or during the course of the treatment.

In certain embodiments, antibody conjugates for diagnostic use m the present application are intended for use in vitro, where the antibody is linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. In certain embodiments, secondary binding ligands are biotin and avidin or streptavidin compounds.

Antibodies of the invention may also be optimized for use in a flow cytometry (FC) assay to determine the activation/phosphorylation status of a target signaling protein in subjects before, during, and after treatment with a therapeutic agent targeted at inhibiting tyrosine, serine and/or threonine phosphorylation at the phosphorylation site disclosed herein. For example, bone marrow cells or peripheral blood cells from patients may be analyzed by flow cytometry for target signaling protein phosphorylation, as well as for markers identifying various hematopoietic cell types. In this manner, activation status of the malignant cells may be specifically characterized. Flow cytometry may be carried out according to standard methods. See, e.g., Chow et al., Cytometry (Communications in Clinical Cytometry) 46: 72-78 (2001).

Alternatively, antibodies of the invention may be used in immunohistochemical (IHC) staining to detect differences in signal transduction or protein activity using normal and diseased tissues. IHC may be carried out according to well-known techniques. See, e.g., Antibodies: A Laboratory Manual, supra.

Peptides and antibodies of the invention may be also be optimized for use in other clinically-suitable applications, for example bead-based multiplex-type assays, such as IGEN, Luminex™ and/or Bioplex™ assay formats, or otherwise optimized for antibody arrays formats, such as reversed-phase array applications (see, e.g. Paweletz et al., Oncogene 20(16): 1981-89 (2001)). Accordingly, in another embodiment, the invention provides a method for the multiplex detection of the phosphorylation state or level at two or more phosphorylation sites of the invention (Table 1) in a biological sample, the method comprising utilizing two or more antibodies of the invention. In certain embodiments the diagnostic methods of the application may be used in combination with other diagnostic tests.

The biological sample analyzed may be any sample that is suspected of having abnormal tyrosine, serine and/or threonine phosphorylation at a novel phosphorylation site of the invention.

In another aspect, the present application concerns immunoassays for binding, purifying, quantifying and otherwise generally detecting the phosphorylation state or level at a novel phosphorylation site of the invention.

Assays may be homogeneous assays or heterogeneous assays. In a homogeneous assay the immunological reaction usually involves a phosphorylation site-specific antibody of the invention, a labeled analyte, and the sample of interest. The signal arising from the label is modified, directly or indirectly, upon the binding of the antibody to the labeled analyte. Both the immunological reaction and detection of the extent thereof are carried out in a homogeneous solution. Immunochemical labels that may be used include free radicals, radioisotopes, fluorescent dyes, enzymes, bacteriophages, coenzymes, and so forth.

In a heterogeneous assay approach, the reagents are usually the specimen, a phosphorylation site-specific antibody of the invention, and suitable means for producing a detectable signal. Similar specimens as described above may be used. The antibody is generally immobilized on a support, such as a bead, plate or slide, and contacted with the specimen suspected of containing the antigen in a liquid phase. The support is then separated from the liquid phase and either the support phase or the liquid phase is examined for a detectable signal using means for producing such signal. The signal is related to the presence of the analyte in the specimen. Means for producing a detectable signal include the use of radioactive labels, fluorescent labels, enzyme labels, and so forth.

Phosphorylation site-specific antibodies disclosed herein may be conjugated to a solid support suitable for a diagnostic assay (e.g., beads, plates, slides or wells formed from materials such as latex or polystyrene) in accordance with known techniques, such as precipitation.

In certain embodiments, immunoassays are the various types of enzyme linked immunoadsorbent assays (ELISAs) and radioimmunoassays (RIA) known in the art. Immunohistochemical detection using tissue sections is also particularly useful. However, it will be readily appreciated that detection is not limited to such techniques, and Western blotting, dot and slot blotting, FACS analyses, and the like may also be used. The steps of various useful immunoassays have been described in the scientific literature, such as, e.g., Nakamura et al., in Enzyme Immunoassays: Heterogeneous and Homogeneous Systems, Chapter 27 (1987), incorporated herein by reference.

In general, the detection of immunocomplex formation is well known in the art and may be achieved through the application of numerous approaches. These methods are based upon the detection of radioactive, fluorescent, biological or enzymatic tags. Of course, one may find additional advantages through the use of a secondary binding ligand such as a second antibody or a biotin/avidin ligand binding arrangement, as is known in the art.

The antibody used in the detection may itself be conjugated to a detectable label, wherein one would then simply detect this label. The amount of the primary immune complexes in the composition would, thereby, be determined.

Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are washed extensively to remove any non-specifically bound labeled secondary antibodies or ligands, and the remaining label in the secondary immune complex is detected.

An enzyme linked immunoadsorbent assay (ELISA) is a type of binding assay. In one type of ELISA, phosphorylation site-specific antibodies disclosed herein are immobilized onto a selected surface exhibiting protein affinity, such as a well in a polystyrene microtiter plate. Then, a suspected neoplastic tissue sample is added to the wells. After binding and washing to remove non-specifically bound immune complexes, the bound target signaling protein may be detected.

In another type of ELISA, the samples are immobilized onto the well surface and then contacted with the phosphorylation site-specific antibodies disclosed herein. After binding and washing to remove non-specifically bound immune complexes, the bound phosphorylation site-specific antibodies are detected.

Irrespective of the format used, ELISAs have certain features in common, such as coating, incubating or binding, washing to remove non-specifically bound species, and detecting the bound immune complexes.

The radioimmunoas say (RIA) is an analytical technique which depends on the competition (affinity) of an antigen for antigen-binding sites on antibody molecules. Standard curves are constructed from data gathered from a series of samples each containing the same known concentration of labeled antigen, and various, but known, concentrations of unlabeled antigen. Antigens are labeled with a radioactive isotope tracer. The mixture is incubated in contact with an antibody. Then the free antigen is separated from the antibody and the antigen bound thereto. Then, by use of a suitable detector, such as a gamma or beta radiation detector, the percent of either the bound or free labeled antigen or both is determined. This procedure is repeated for a number of samples containing various known concentrations of unlabeled antigens and the results are plotted as a standard graph. The percent of bound tracer antigens is plotted as a function of the antigen concentration. Typically, as the total antigen concentration increases the relative amount of the tracer antigen bound to the antibody decreases. After the standard graph is prepared, it is thereafter used to determine the concentration of antigen in samples undergoing analysis.

In an analysis, the sample in which the concentration of antigen is to be determined is mixed with a known amount of tracer antigen. Tracer antigen is the same antigen known to be in the sample but which has been labeled with a suitable radioactive isotope. The sample with tracer is then incubated in contact with the antibody. Then it can be counted in a suitable detector which counts the free antigen remaining in the sample. The antigen bound to the antibody or immunoadsorbent may also be similarly counted. Then, from the standard curve, the concentration of antigen in the original sample is determined.

In another aspect, the invention relates to a kit comprising a reagent capable of determining the level of phosphorylation in residues of interest of transferrin protein for determining the risk of a subject developing Alzheimer's or a cognitive disorder similar to Alzheimer's disease, for designing a personalized therapy in a subject or for selecting a patient susceptible to be treated with a therapy for the prevention and/or treatment of Alzheimer's disease or a cognitive disorder similar to Alzheimer's disease.

As it is used herein, “kit” refers to a product containing the different reagents required for carrying out the methods of the invention packaged so as to allow their transport and storage. Materials suitable for packaging the components of the kit include glass, plastic (polyethylene, polypropylene, polycarbonate and the like), bottles, vials, paper, sachets and the like. Additionally, the kits of the invention can contain instructions for the simultaneous, sequential or separate use of the different components in the kit. Said instructions can be found in the form of a printed material or in the form of an electronic support capable of storing instructions such that they can be read by a subject, such as electronic storage media (magnetic discs, tapes and the like), optical media (CD-ROM, DVD) and the like. Additionally or alternatively, the media can contain Internet addresses which provide said instructions.

As it is used herein, “reagent capable of determining the level of phosphorylation” is understood to be a compound capable of detecting a phosphorylated residue of a protein.

In a particular embodiment, the reagent capable of determining the level of phosphorylation of transferrin protein is selected from the group consisting of
a) a reagent capable of determining the level of phosphorylation in residue of interest of human transferrin protein or of a functionally equivalent variant in a phosphorylatable, positionally equivalent ammo acid residue of another transferrin protein as defined by multiple amino acid sequence alignment,

In another embodiment, the kit of the invention comprises one or more reagents mentioned above.

Additionally, the kit of the invention comprises a reagent which is capable of binding specifically to transferrin protein. In a more particular embodiment, said reagent is an antibody.

As it is used herein, “specific recognition or specific binding,” when referring to a peptide or protein with a phosphorylated residue, refers to the fact that said reagent only recognizes the peptide or protein when it is phosphorylated in the residue of interest and does not exhibit any reaction when it is not phosphorylated. When referring to a peptide or protein irrespective of its level of phosphorylation, it refers to the fact that the reagent is capable of reacting with at least one epitope of the peptide or protein, in contrast with a non-specific interaction.

As it is used herein, the term “antibody” can be a natural polyclonal or monoclonal antibody or a non-natural antibody, for example, a single-domain antibody, a single-chain variable-fragment antibody, a microantibody, etc. Methods for producing such antibodies are well known in the art.

In some embodiments, the specific antibodies used in the invention are labeled with a detectable marker (for example, a fluorescent dye or a detectable enzyme), or modified to make detection easier (for example, with biotin to allow for detection with an avidin or streptavidin). In other embodiments, the reagent will not be directly labeled or modified.

In certain embodiments, the kits include the reagents in the form of an array. The array includes at least two different reagents suitable for determining the levels of phosphorylation in one or more residues of interest bound to a substrate in a predetermined pattern (for example, a grid). The present invention therefore provides arrays comprising the reagents suitable for determining the levels of phosphorylation of one or more amino acid residues mentioned in the invention.

The placement of the different reagents (the “capture reagents”) allows measuring the levels of phosphorylation of a number of different amino acid residues in the same reaction. Kits including reagents in array form are usually found in sandwich format, so such kits can also contain detection reagents. Different detection reagents are usually included in the kit, where each detection reagent is specific for a different antibody. The detection reagents in such embodiments are usually reagents specific for the same proteins as the reagents bound to the substrate (although the detection reagents typically bind to a different portion or in the protein site of the substrate-bound reagents), and are generally affinity-type detection reagents. Like the detection reagents of any other assay format, the detection reagents can be modified with a detectable residue, modified to allow the separate binding of a detectable residue, or they may not be modified. Array-type kits including detection reagents which are modified or not modified to allow the binding of a detectable residue can also contain additional detectable residues (for example, detectable residues that bind to the detection reagent, such as labeled antibodies binding without modifying detection reagents or streptavidin modified with a detectable residue for biotin detection, modified detection reagents).

The antibodies can be brought about by means of methods known in the art. For example, a mammal such as a mouse, a hamster or a rabbit can be immunized with an immunogenic form of an transferrin protein phosphorylated in a specific residues of interest (for example, antigenic fragment which can bring about an antibody response, for example a synthetic peptide containing the phosphorylated amino acid). Techniques for conferring immunogenicity to a protein or peptide include vehicle conjugation or other techniques that are very well known in the art. For example, a peptidyl portion of a polypeptide can be administered in the presence of an adjuvant. The progression of immunization can be monitored by detecting plasma or serum antibody titers. Standard ELISA or other immunoassays can be used with the immunogen as an antigen for evaluating the levels of antibodies.

After immunization, antisera that are reactive with a polypeptide can be obtained, and polyclonal antibodies can be isolated from the serum if desired. To produce monoclonal antibodies, antibody-producing cells (lymphocytes) can be collected from an immunized animal and fused using standard methods for fusing somatic cells with immortalizing cells to give rise to hybridoma cells. Such techniques are very well known in the art and include, for example, the hybridoma technique, such as the human B-cell hybridoma technique and the EVB hybridoma technique for producing human monoclonal antibodies. Hybridoma cells can be immunochemically screened for producing antibodies that are specifically reactive with the polypeptides and isolated monoclonal antibodies.

In another more particular embodiment, the reagent which is capable of binding specifically to transferrin protein is immobilized on a support.

The terms described above are likewise applicable to this aspect.

In another aspect, the invention relates to the use of a kit of the invention for determining the risk of a subject developing Alzheimer's disease or a cognitive disorder similar to said disease in a subject, for designing a personalized therapy in a subject suffering from mild cognitive impairment or for selecting a patient susceptible to be treated with a therapy for the prevention and/or treatment of Alzheimer's or a cognitive disorder similar to said disease.

Antibodies and peptides of the invention may also be used within a kit for detecting the phosphorylation state or level at a novel phosphorylation site of the invention, comprising at least one of the following: an antibody or an antigen-binding fragment thereof that binds to an amino acid sequence comprising the phosphorylation site of transferrin. Such a kit may further comprise a packaged combination of reagents in pre determined amounts with instructions for performing the diagnostic assay. Where the antibody is labeled with an enzyme, the kit will include substrates and co-factors required by the enzyme. In addition, other additives may be included such as stabilizers, buffers and the like. The relative amounts of the various reagents may be varied widely to provide for concentrations in solution of the reagents that substantially optimize the sensitivity of the assay. Particularly, the reagents may be provided as dry powders, usually lyophilized, including excipients that, on dissolution, will provide a reagent solution having the appropriate concentration.

According to one aspect of the invention, there is provided a method for screening an individual who is at risk of dementia for dementia diagnosis comprising:
providing a sample from the individual; measuring a level of at least one isoelectric point fraction of transferrin in the sample; and comparing the sample level of the at least one isoelectric point fraction to a control level of the at least one isoelectric point fraction from a healthy individual, wherein for a positive result, the sample level and the control level are different.

According to an aspect of the invention, there is provided a method for screening an individual who is at risk of dementia for dementia diagnosis comprising:
providing a sample from the individual; determining a transferrin profile of the sample; and comparing the transferrin profile of the sample to a reference value, wherein for a positive result, the transferrin profile of the sample and the reference value are different.

As discussed herein, the inventor has demonstrated that samples from both Early Onset Alzheimer's disease and Late Onset Alzheimer's disease have an abnormal transferrin profile. While not wishing to be bound to a particular theory or hypothesis, it is —believed that the abnormal transferrin profile is caused either by reduced phosphorylation of transferrin, for example in a situation wherein CaMKK2 or another kinase, for example, a kinase such as CaMK4 which is downstream of CaMKK2, is dysfunctional or possibly by an activated phosphatase.

In addition to the effect of phosphorylation on pl, acetylation will also cause a shift to a negative pI.

As discussed herein, when focusing on an IPG pH 3-10 strip, there are three major pl groups for transferrin: at pI/pH˜9-10 (basic), pI/pH˜5-6(neutral) and pI/pH˜3-4 (acidic). As will be appreciated by one of skill in the art, different subfractions within each group may be resolved by using narrower pH range strips. As discussed below, a specific fraction or subfraction may be measured or the transferrin profile may be determined.

As discussed herein, determining the transferrin profile to compare the transferrin profile of an individual at risk of dementia to a reference value requires either measurement and/or visualization of one or more isoelectric forms of transferrin. As discussed herein, the profile may be a profile or measurement or visualization of one or more charged fractions of transferrin.

As will be appreciated by one of skill in the art, a transferrin profile may take many different forms and still be considered a “transferrin profile” as used herein.

For example, as discussed herein, a suitable sample may be subjected to pl separation and immunoblotted with one or more anti-transferrin antibody/antibodies.

As will be appreciated by one of skill in the art, in a transferrin profile generated in this manner, the number of fractions will depend on the nature of the separation that the sample is subjected to, as discussed herein.

Yet further, the fraction measured or visualized does not necessarily need to be the acidic (pH 3-4) fraction but may be one of the other fractions or subfractions. In preferred embodiments, the protein concentration of the sample from the test individual or individual of interest and the protein concentration of the control sample from the healthy individual are known or more preferably approximately balanced.

As will be appreciated by one of skill in the art, in this manner, one or more fractions containing one or more isoelectric forms of transferrin can be analyzed, as discussed herein, so as to provide a transferrin profile.

As used herein, “isoelectric form of transferrin” refers to a post translational modified form of transferrin that is phosphorylated/unphosphorylated at specific residues and/or acetylated/unacetylated at specific residues. As will be appreciated by one of skill in the art, the “simplest” isoelectric form of transferrin is unphosphorylated and unacetylated while other isoelectric forms will be phosphorylated at one or more residues and/or acetylated at one or more residues and will have a specific isoelectric point. As will be appreciated by one of skill in the art, some isoelectric forms may have a similar pl and accordingly may be detected or visualized as part of the same fraction on IPG when determining the transferrin profile of a sample.

In other embodiments of the invention, the modification state at a particular amino acid residue of transferrin may be determined or queried or the number of peptides having that particular modification state at that particular residue(s) may be determined or measured. As will be appreciated by one of skill in the art, this represents another method for measuring at least one transferrin isoelectric point fraction and/or determining the transferrin profile from a sample.

In some embodiments, the transferrin profile or level of a transferrin isoelectric point fraction is determined by determining the modification state at one or more amino acid residues of transferrin selected from the group consisting of: K359; K37; K508; K546; S136; S144; S298; S305; S306; S389; S468; S520; S63; S688; T139; T340; T349; T355; T36; T476; T537; T654; T686; T694; Y155; Y207; Y257; Y533; Y534; Y536; Y593; Y64; Y666; Y669; and Y674.

As will be known by those of skill in the art, specific antibodies can be generated to detect acetylation/phosphorylation at a particular residue. The antibodies to detect potential phosphorylation/acetylation residues can be made by following 2 approaches:

### Approach-1: Monoclonal Antibody:

The peptide sequence, approximately 15 amino acid residues (Kringelum et al., 2013), encompassing the potential phosphorylated/acetylated resides in TF will be evaluated for antigenic potential using linear epitope prediction models, for example BepiPred™ (Larsen et al., 2006). Alternatively, conformational epitope modelling will be evaluated for antigenic potential using bioinformatics tools, for example DiscoTope™ 2.0 (Haste Andersen et al., 2006; Kringelum et al., 2012).
The synthetic peptide with appropriate adjuvants will be used for immunization in mouse.
Following immunizations, the lymphocytes will be harvested from spleen or lymph node and subsequently immortalized using myeloma cells (e.g. X63.Ag8.653 cell line). The hybridomas will be selected and the supernatant will be screened by ELISA or WBLOT for antigen detection.
Selected hybridoma will be expanded and further characterized for isotype determination, epitope mapping, specificity and sensitivity. The hybridoma will also be characterized for expression, solubility, stability, affinity and avidity of the monoclonal antibody.
The appropriate hybridoma producing desired antibody will be expanded for mass production. The monoclonal antibody will then be tested for detection of the pH3-4 fraction of TF and used for diagnostic kit preparation.

### Approach-2: Screening Phage Display Library:

We will generate acetylated or phosphorylated peptides (mentioned above) specific single chain variable fragment (scFv) or antigen-binding fragment (Fab) antibodies by screening commercially available human scFv libraries, human Fab libraries, mouse scFv/Fab libraries, rabbit Fab libraries, chicken scFv libraries and single domain antibody libraries.

These modification-specific antibodies may be used to detect specific isoelectric forms. Alternatively, other anti-transferrin monoclonal antibodies or anti-transferrin polyclonal antibodies may be used in approaches to identify and quantify an isoelectric fraction of transferrin, for example, the pH-3-4 fraction, as discussed herein.

### Approach-1: Isoelectric Focusing and SDS-PAGE Followed by Immunoblotting:

Serum/CSF proteins will be precipitated and then dissolved in urea followed by focusing on immobilized polyacrylamide gradient gel strips containing linear or nonlinear pH gradients 3-10/3-7/3-6.
Alternatively, serum/CSF proteins will be treated with Deglycosylase (to remove Nand O-linked glycans) and hydroxylamine (to remove thioesters). After treatment, the [proteins will be precipitated and focused.
Following first dimension separation of the protein based on charge, proteins will be further separated in 2nd dimension based on molecular weight in using denaturing SDS-PAGE. The 2nd dimension separated proteins will then be reduce and alkylated and transferred to nitrocellulose/PVDF membrane and immunoblotted using anti-transferrin or anti-P-transferrin specific antibodies and compared to standard age matched healthy persons fractionated transferrin profile.

### Approach-2: Gel-Free Pre Fraction of Serum/CSF Protein Using Pl-TRAP Followed by Immunoblotting or Mass Spectrometric Identification:

Serum/CSF protein will be desalted and fractionated by a gel free, in solution isoelectric focusing method using pI-TRAP (Biomotif).
30 fractions of different pI values will be collected and the presence of transferrin in those fractions will be quantified by ELISA, immunoblotting or a Mass spectrometry based method.

### Approach-3: Surface Plasmon Resonance (SPR) Based Detection of Phospho-Transferrin On-Chip:

The specific phospho-transferrin proteins will be detected and quantified and compared in serum/CSP by SPR based technology using microfluidic sensor-chips coated with anti-transferrin antibodies.

### Approach-4: Immunoassay.

Solid-phase enzyme immunoassay will be developed for the quantitative determination of PTF in human CSF or serum

The invention will now be described by way of examples. However, the invention is not necessarily limited to the examples.

### Example 1—Protein Profiling and Mass-Spectrometry Based Study Revealed CaMKK2 Knockdown in DRG Neurons Affected P-TF

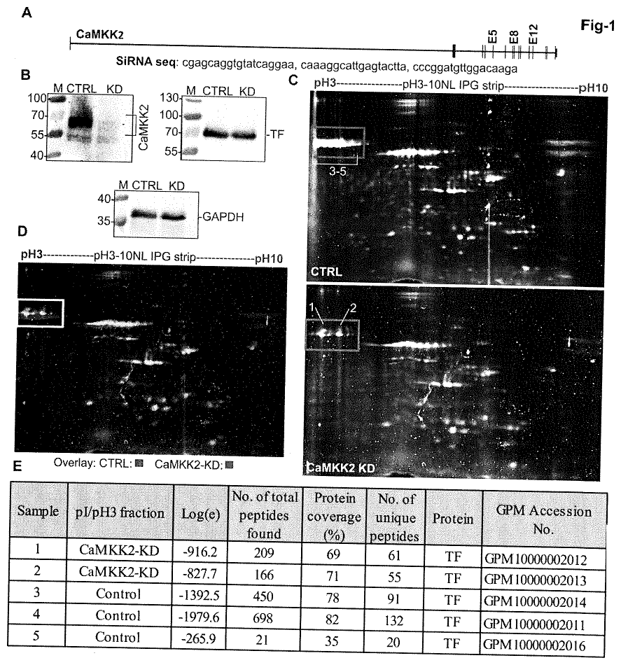

In order to understand the role of CaMKK2 in peripheral neurons, we knocked down CaMKK2 in cultured adult primary rat DRG neurons and fractionated total cellular proteins in the 1^{st} dimension IEF followed by 2^{nd} dimension SDS-PAGE to find out differentially charged protein fractions (FIG.-1A-D). Comparison of the focused proteins revealed reduced abundance of multiple ˜75 kDa focused spots at pH-3-4, which was then analyzed by in-gel tryptic digestion followed by mass-spectrometric identification of the proteins (FIG. 1 C-E, marked spots in rectangle area). Mass spectrometry identified the spots as multiple P-TF (FIG. 1 E). The potential P-TF residues in the control (scrambled siRNA) are P-Tyr257/333/336/338, P-Ser381/389/409/500/511/512 and P-Thr392/393/586 respectively, which was found absent or reduced in CaMKK2 knockdown (Table 1).

### Example 2—Immunoblotting Using Anti-TF Antibody Confirmed that Loss of CaMKK2 Affects TF Phosphorylation

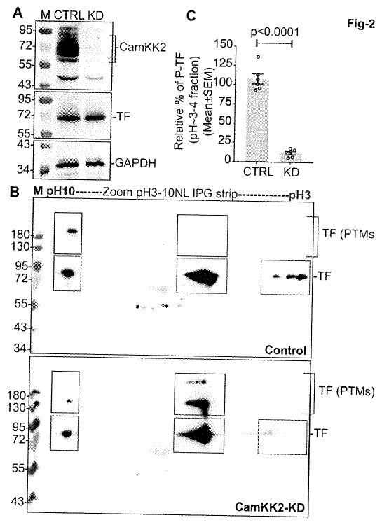

Molecular weight and pI of human/rat TF (SwissPort: P02787/P12346) is 77/76 kDa and 6.81/7.14 respectively. The defined DRG neuron culture media was supplemented with partially saturated recombinant human TF (71-81 kDa). Therefore, we would expect a mixture of cross species TF in our protein analysis. Relative amount of TF was unaltered but charged fractions of TF differed in CaMKK2 knockdown DRG neurons (FIG. 2AB). In 2D IEF/SDSPAGE, TF appeared as 3 major fractions at pH˜3-4, ˜5-6 and ˜9-10 respectively (FIG. 2B, red rectangles). The pH-3-4 fractions of TF correspond to multiple phosphorylated residues that was previously analyzed by MS-MS and found significantly decreased in CaMKK2 knockdown neurons (FIG. 2BC, red rectangle). This confirms the protein profiling and mass spectrometry-based findings shown in FIG. 1. In addition, CaMKK2 knockdown DRG neurons exhibited presence of additional focused spots at ˜130/>180 kDa and at pH˜5-6 and pH-9-10 regions whereas scrambled control cells exhibited >180 kDa at pH˜9-10 fraction only (FIG. 2B, blue rectangles). The high molecular weight forms may be due to post translation modifications that added additional molecular mass, for example glycosylation, and that appeared to be controlled by CaMKK2 loss of function.

### Example 3—Loss of CaMKK2 Reduced Abundance and Phosphorylation of TF in CaMKK2 KO Mice DRG Tissues

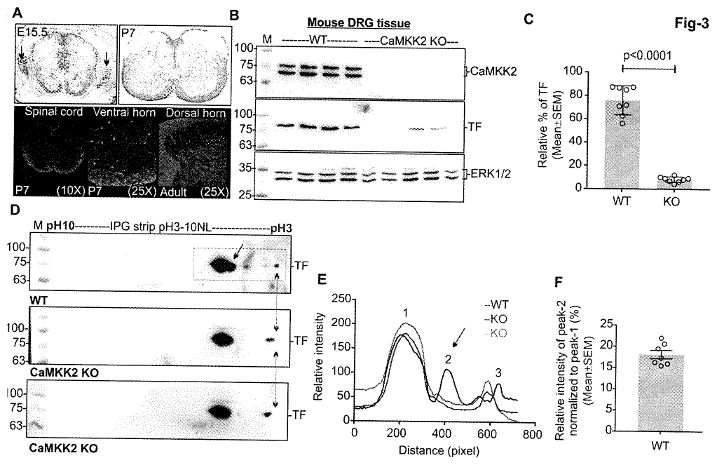

TF promoter-trapped GFP reporter expression showed CaMKK2 is expressed in the mouse spinal cord neurons (FIG. 3A). Immunoblotting revealed expression of CaMKK2 isoforms equivalent to 75 and 70 kDa proteins in the DRG tissues that are absent in the KO mice (FIG. 3B). Immunoblotting based quantification revealed that the relative amount of TF is significantly low in CaMKK2 KO DRG tissues (FIG. 3BC). 2D IEF/SDS-PAGE revealed that TF pH-3-4 fractions (P-TF) significantly differed between wild type and CaMKK2 KO mice DRG tissues (FIG. 3D-F).

### Example 4—Loss of CamKK2 Affected Relative Abundance and Phosphorylation of TF in a Tissue Specific Manner

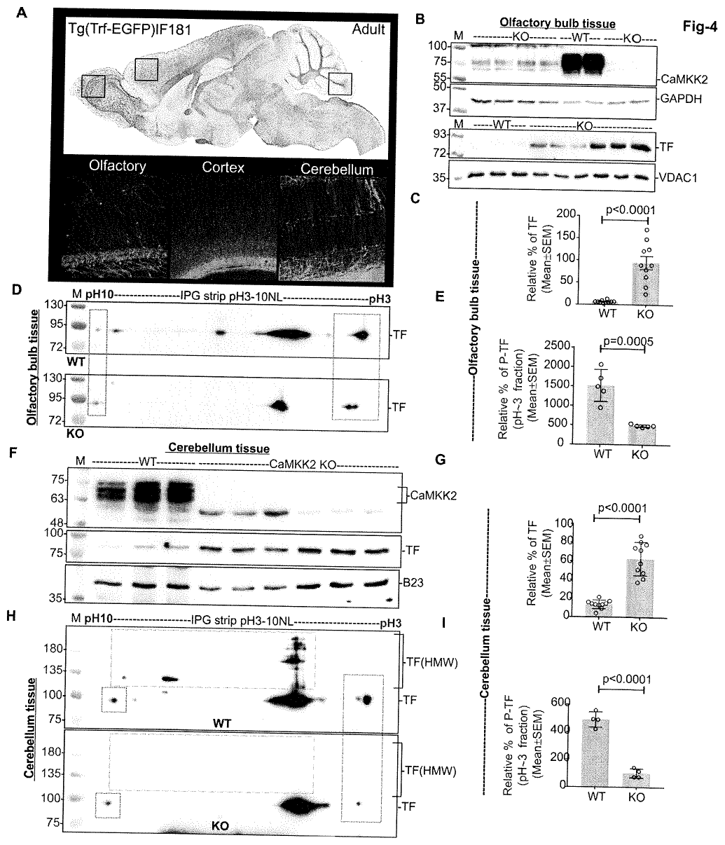

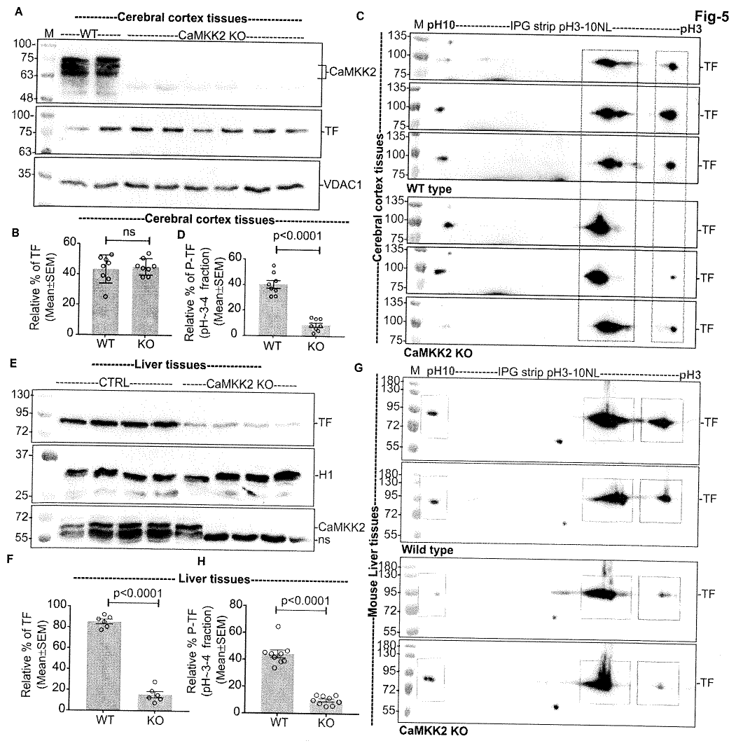

TF promoter-trapped GFP reporter mouse revealed that TF is expressed in the neurons of olfactory bulb, cortex and cerebellum (FIG. 4A). Immunoblotting based quantification revealed significantly high levels of TF in CaMKK2 KO olfactory bulb and cerebellum (FIG. 4BC & FG). IEF/SDS-PAGE revealed significantly decreased amounts of P-TF (pH-3-4 fractions) in the CaMKK2 KO olfactory bulb, cerebral cortex, cerebellum and liver tissues (FIG. 4DE & HI, 5CD & GH). Loss of CamKK2 had no effect on relative amount of TF in the cortex (FIG. 5AB). However, loss of CaMKK2 significantly decreased the relative amount of TF in the liver (FIG. 5EF).

### Example 5—Knockdown of CaMKK2 Affected Vesicular Trafficking of TF in Cultured Adult Rat DRG Neurons

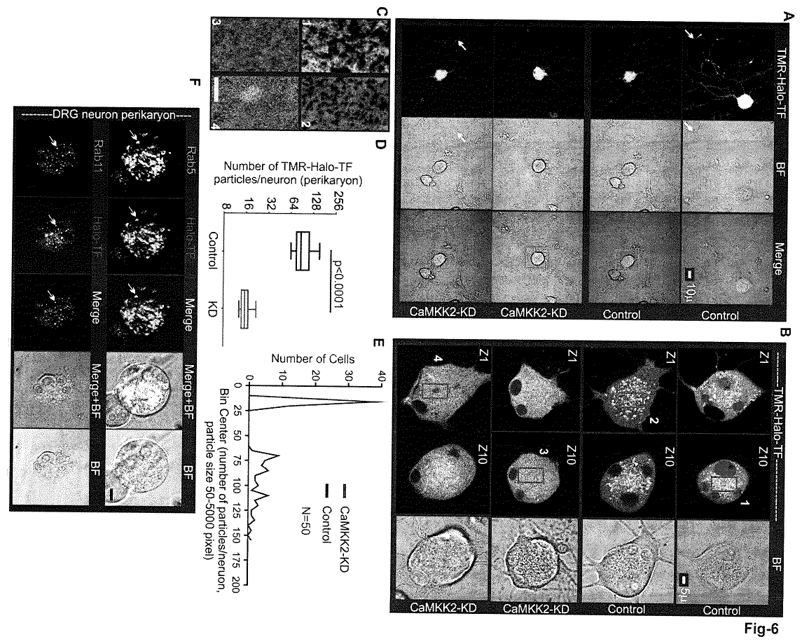

Confocal live cell imaging of Halo-tagged TF (Halo-TF) expressed neurons revealed Halo-TF is distributed in the perikaryon as well as in the neurites (FIG. 6A). High magnification image of the perikaryon revealed distinct vesicular structures (particles) containing high amount of TMR labelled Halo-TF in the control (scrambled). The vesicular structures were found significantly less abundant m CaMKK2 knockdown cells (FIG. 6B-E). Immunofluorescence revealed that majority of the Halo-TF associated vesicular structures are Rab5-positive early endosomes and a considerable fraction is Rab11-positive recycling endosomes (FIG. 6F) (Mills et al., 20 1 0). The deficiency of Halo-TF associated vesicular structures in CaMKK2 knockdown neurons indicates impaired intracellular trafficking.

### Example 7—Negative Charged Fractions of CaMKK2 and TF (Phosphorylation) are Significantly Reduced in Early and Late-Onset 3xTg-AD Mouse Cerebral Cortex

Dysregulation of the intracellular Ca²⁺ homeostasis is an underlying factor for the development of AD (Hermes et al., 2010; Berridge, 2011). Transgenic 3xTg-AD mouse was used to study the negative charged fractions of CaMKK2 and TF (P-CaMKK2 and P-TF) during progression of AD. Only female 3xTg-AD mouse was used to avoid gender reported differences in neuropathology and behavior (Hirata-Fukae et al., 2008; Gimenez-Llort et al., 2010; Garcia-Mesa et al., 2011; Hebda-Bauer et al., 2013). Progressive increase in the Aβ peptide deposition was detected in some brain regions of 3xTg-AD mice as early as 3-4 months (Oddo et al., 2003). Synaptic transmission and long-term potentiation were impaired at 6 months in 3xTg AD mice (Oddo et al., 2003). Conformationally altered and hyperphosphorylated tau were detected in the hippocampus of 3xTg-AD mice at 12-15 months (Oddo et al., 2003). Therefore, 6 months (early) and 14 months (late) were considered as early and late stage of 3xTg-AD mouse model and studied.

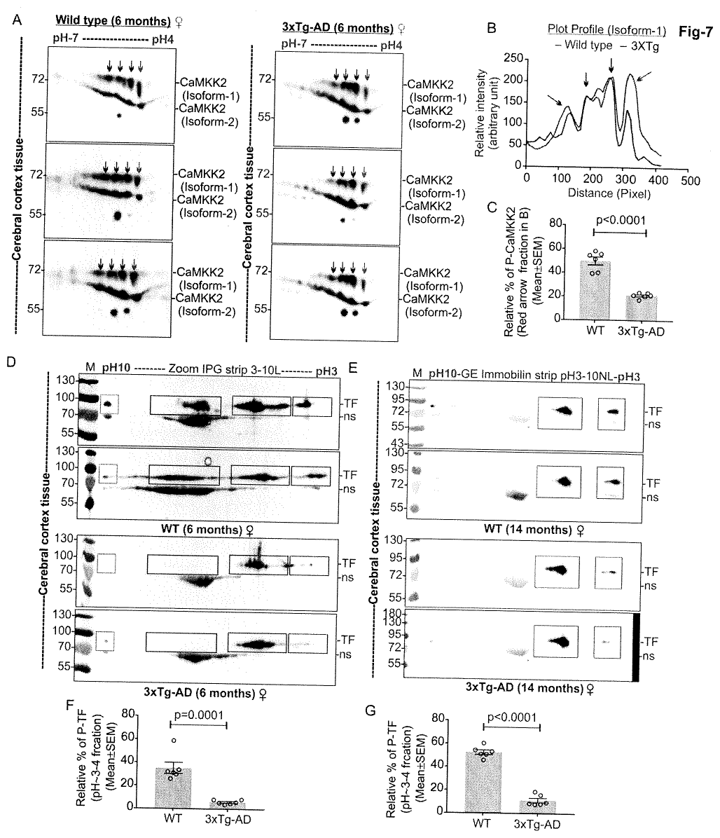

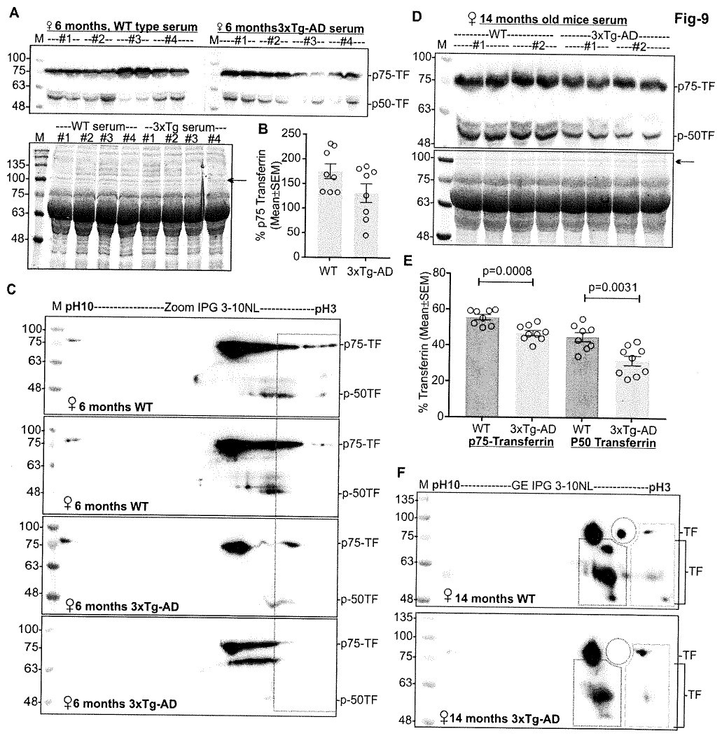

The molecular weight and pl of the mouse CaMKK2 isoforms (transcript ID: ENSMUST00000111668.7 and ENSMUST00000200109.4 respectively) are predicted as 73/59 kDa and 5.27/5.3 1 respectively by ExPASy-Compute pI/MW tool (Gasteiger et al., 2003). IEF/SDS-PAGE of early 3xTg-AD and age matched wild type cortex tissues revealed presence of ˜73 kDa and ˜59 kDa CaMKK2 proteins corresponding to isoform-1 and -2 respectively (FIG. 7 A). IEF/SDS-PAGE also revealed CaMKK2 isoforms-1 is differentially charged in early 3xTg-AD cortex (FIG. 7 A, red and blue arrows). Previous study using IEF/SDS-PAGE analysis of immunoprecipitated CaMKK2 from mammalian cells revealed that the multiple phosphorylated spots observed in our studies appeared to be positive for P-Ser and mutation of S129A, S133A and S137A lead to disappearance of majority of the spots (Green et al., 2011). Validated P-CaMKK2 antibodies are not available. Therefore, we considered the more negative charged fraction of CaMKK2 as P-CaMKK2 and relative quantification revealed significant reduction of P-CaMKK2 (red arrow marked fraction) in the early 3xTg-AD cortex (FIG. 7 A-C). TF in early 3xTg-AD cortex appeared as 4 major charged fractions at pH˜10, 7-8, 5-6, and 3-4 respectively (FIG. 7D, colored rectangles). In early 3xTg-AD cortex, TF pH-3-4 fractions were significantly decreased (FIG. 9DF). In contrast, TF appeared as 2 major fractions at pH˜5-6 and ˜3-4 respectively, in 14 months old mouse cortex (FIG. 7E). The pH-3-4 fraction of TF was significantly reduced in late 3xTg-AD cortex (FIG. 7G).

### Example 8—CaMKK2 KO and 3xTg-AD Exhibited Altered CaMKK2 and TF Associated Multiprotein Complexes

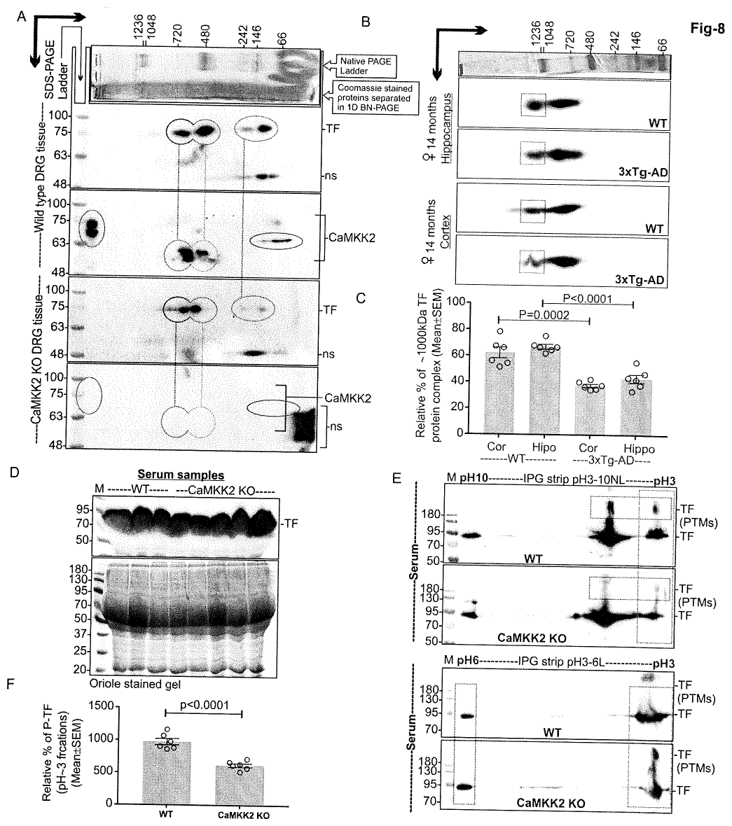

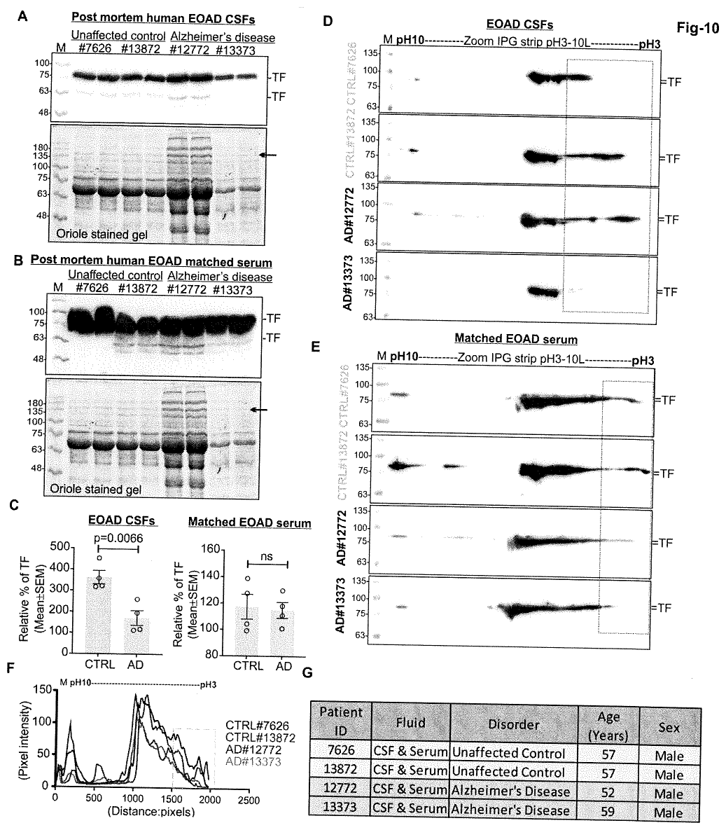

Two dimensional BN-PAGE/SDS-PAGE was used to study TF and CaMKK2 associated protein complexes. We hypothesized that reduced phosphorylation may affect the dynamics of TF/CaMKK2 associated protein complexes due to recruitment/dissociation of interacting proteins. BN-PAGE/SDS-PAGE revealed TF formed 4 major protein complexes at ˜720, ˜480, ˜242 and ˜100 kDa respectively in the wild type DRG tissues (FIG. 8A; blue, green and orange circles respectively). The CaMKK2 isoforms formed 4 protein complexes at >1200, ˜720, ˜480 and ˜66 kDa respectively (FIG. 8A; orange, blue, green and pink circles respectively). The CaMKK2 isoform-1 (˜73 kDa) formed >1200 and ˜66 kDa complex whereas, CaMKK2 isoform-2 (˜59 kDa) formed ˜720 and ˜480 kDa complexes (FIG. 10A). The >1200, ˜720 and ˜480 kDa CaMKK2 complexes appeared to contain higher molecular weight forms which may be due to PTMs of a fraction of proteins in the respective complex. In BN-PAGE/SDS-PAGE analysis, the interacting proteins in the same complex appear on a vertical line. The vertical alignments of the ˜720 and ˜480 kDa CaMKK2 complexes with TF associated complexes indicate possible association of these proteins in the same complex (FIG. 8A, blue and green dotted lines). This also indicates the possibility that CaMKK2 isoform-2 may be involved in TF phosphorylation. Interestingly, in CaMKK2 KO DRG tissue all CamKK2 protein complexes disappeared as expected. In addition, in the CaMKK2 KO DRG tissue, the ˜720 and ˜480 kDa TF associated protein complexes shifted their relative position, and the ˜242 and ˜100 kDa TF complexes disappeared, which indicates the dynamics these complexes are dependent on CaMKK2.

We studied the dynamics of TF associated protein in late 3xTg-AD cerebral cortex and hippocampus tissues. TF appeared as 2 major complexes at ˜1000 and ˜720 kDa respectively in cortex and hippocampus (FIG. 8B). The ˜1000 kDa TF associated protein complex was significantly lower in the hippocampus and cortex of late 3xTg-AD mice (FIG. 8B (red rectangle) and 8C). This indicates that reduced P-CaMKK2 in 3xTg-AD mice brain lead to decreased P-TF which affected dynamics of the TF associated protein complexes.

### Example 9—Reduced P-CaMKK2 in 3xTg-AD Affected Serum TF Abundance and Phosphorylation

TF produced and secreted from brain enter systemic circulation, therefore, we hypothesized that reduced P-TF in CaMKK2 KO and 3xTg-AD mice brain may be reflected in the reduced serum P-TF level, which in turn may serve as a serum based minimal-invasive biomarker for AD. Relative abundance of TF remained unaltered in CaMKK2 KO mice and early 3xTG-AD but significantly reduced in late 3xTg-AD serum (FIG. 8D, 9A-E). TF appeared in serum appeared as 75 and 50 kDa proteins, p75-TF and p50-TF respectively. IEF/SDSPAGE revealed significantly less P-TF (pH-3-4 fraction) in CaMKK2 KO serum (FIG. 8E-F) and comparatively less pH-3-4 P-TF in both early and late 3xTg AD serum (FIG. 9CF). This indicates that serum TF level does not reflect the physiological state in CaMKK2 KO mice but the phosphorylation level may reflect that. Similarly, serum TF level at early stage of AD does not reflect the disease state but P-TF may provide accurate prediction of AD.

### Example 10—Relative Abundance and Phosphorylation of TF is Altered in the CSF and Serum from Early and Late-Onset Human Postmortem AD Patients

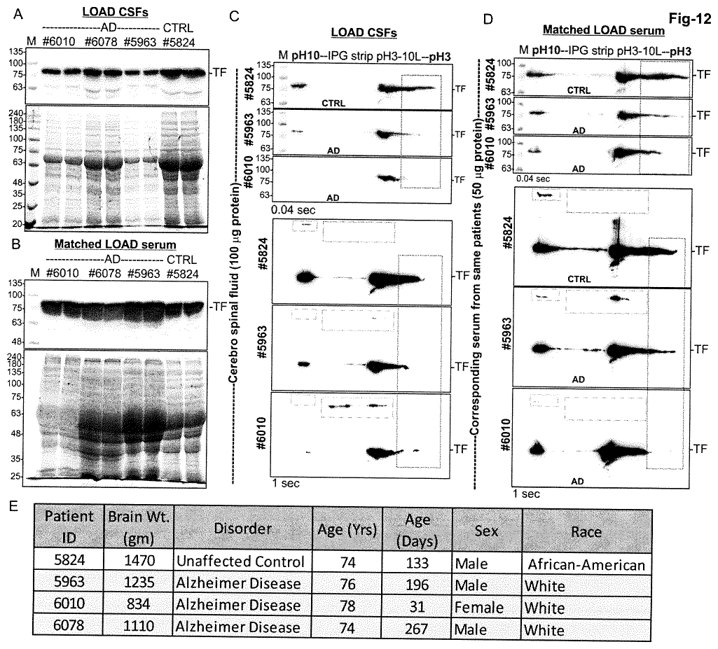

We analyzed relative abundance and phosphorylation (pH-3-4 fraction) of TF in postmortem human CSF and serum samples derived from early-onset AD (EOAD<65 yrs) and late-onset AD (LOAD>65 yrs) (Tellechea et al., 2018) to test the applicability of P-TF as a diagnostic and prognostic marker. Relative abundance of TF was significantly reduced in the CSF from EOAD patients but the serum level remained unaffected (FIG. 10A-C). IEF/SDSPAGE of 7 EOAD CSF samples revealed comparatively reduced pH-3-4 fraction of TF, except one EOAD sample (patient ID: 12772) (FIG. 10D & 11C). Interestingly, in 2 EOAD patients, the matched serum was available and IEF/SDS-PAGE detected comparative loss of TF pH-3-4 fractions (FIGS. 10E & F, dotted rectangles). This indicates that P-TF fraction in human serum may detect EOAD in human and may serve as an early diagnostic biomarker for AD. The abundance of TF was comparatively reduced in LOAD CSF but the serum level remained unaffected (FIG. 12AB). IEF/SDS-PAGE revealed comparative reduction of TF pH-3-4 fractions in both serum and CSF. This indicates that P-TF fraction-based biomarker may serve as prognostic biomarker for AD as well.

### Materials and Methods:

Transgenic Mouse (CaMKK2 KO and 3xTg-AD) and Postmortem Human Extracellular Fluids from Alzheimer's Patients:

The CaMKK2 KO mouse brain and spinal cords were provided as dissected snap-frozen tissue by Dr. Uma Sankar, Indiana University School of Medicine, USA. The CaMKK2 knockout mouse was generated by targeted deletion of exons 2-4 flanking sequence (Anderson et al., 2008). The 3xTg-AD mouse cerebral cortex, hippocampus and serum samples were provided by Dr. Benedict C Albensi, University of Manitoba, Canada. The 3xTg-AD is a triple-transgenic model of AD harboring PS 1 (M146V), APP(Swe) and tau (P301L) trans genes (Oddo et al., 2003). The postmortem human cerebrospinal fluid (CSF) and matched serum samples from Alzheimer's patients and unaffected controls were obtained through NIH NeuroBioBank (Request #937) (Table 51).

### Cell Culture

DRG from adult male Sprague-Dawley rats were dissected and dissociated as described previously and cultured in defined Hams F 12 media containing 10 mM D-glucose (N4888, Sigma, St Louis, Mo., USA) supplemented with modified Bottenstein's N2 additives without insulin (0.1 mg/ml TF, 20 nM progesterone, 100 μM putrescine, 30 nM sodium selenite, 0.1 mg/ml BSA; all additives were from Sigma, St Louis, Mo., USA) (Akude et al., 2011; Roy Chowdhury et al., 2012; Saleh et al., 2013; Calcutt et al., 2017). In all experiments, the media was also supplemented with 0.146 g/L L-glutamine, a low dose cocktail of neurotrophic factors (0.1 ng/ml NGF, 1.0 ng/ml GDNF and 1 ng/ml NT-3—all from Promega, Madison, Wis., USA), 0.1 nM insulin and IX antibiotic antimycotic solution (A5955, Sigma).

### Knockdown of CaMKK2:

Knockdown of CamKK2 in cultured DRG neurons was achieved by 2 methods. In protein profiling experiment, knockdown was performed by transfecting cells with lipid nanoparticles (LNP) encapsulated cocktail of 3 siRNAs specific to exon 5, 8 and 12 (s135956, s135958 and s135957) of CaMKK2 gene respectively (Rungta et al., 2013). The siRNA-LNPs are prepared by mixing appropriate volumes of different cationic lipid stock solutions in ethanol with an aqueous phase containing siRNA multiplexes using a microfluidic micromixer by Precision NanoSystems Inc. For encapsulation, desired amount of siRNAs (0.056 mg siRNA/micromole of lipid) was dissolved in the formulation buffer (25 mmol/L sodium acetate, pH 4.0). Subsequently, 1× volume of the lipid mixtures in ethanol and 3× volumes of the siRNA in formulation buffer were combined in the microfluidic micromixer using a dual syringe pump to generate the LNPs. The LNP particles containing siRNA were added to DRG culture and neurons were allowed to grow for 48 hours, after which the proteins were analyzed. In all other experiments, the siRNAs were transfected in freshly dissociated DRG neurons using the rat neuron nucleofection kit (VPG-1003, Amaxa, Lonza Inc., Allendale, N.J., USA) and Amaxa nucleofector-II apparatus (program 0-003) and cultured in poly L-Ornithine (P8638, Sigma) and laminin coated μ-Plate-24 well (Ibidi USA, Inc. Madison, Wis., USA).

### Two Dimensional Isoelectric Focusing and SDS-PAGE:

IEF separates protein based on isoelectric point (pl) which depends on net charge in the protein. During focusing, the proteins will migrate to the point on an immobilized pH gradient (IPG) where the net charge of the protein is zero (Freeman and Hemby, 2004). The charge separated proteins were further separated in the 2^{nd} dimension SDS-PAGE based on their molecular weight. Total cellular proteins were precipitated and dissolved in rehydration buffer containing 8 M Urea, 2% CHAPS, 50 mM dithiothreitol (DTT) and 0.2% Bio-Lyte ampholytes pH3-10. The dissolved proteins were then incubated in IPG strips (ThermoFisher) for 1 hand focused at 175 volt (V) for 15 min, 175-2000V ramp for 45/109 min (depending on pH gradient) and 2000V for 30 min. In some experiments, IPG strips from GE Healthcare Life Sciences (Immobiline DryStrip™ pH 3-10 L) and Biorad (ReadyStrip™ IPG strips pH 4-7 L) were used and the proteins were focused using Biorad Protean®i 12 IEF™ system as per manufacturer's recommendations. After focusing the proteins in the strips were reduced (by DTT), alkylated (by Iodoacetamide) and resolved on 2D SDS-PAGE. For protein profiling experiments, the gel was stained with colloidal coomassie, imaged and the protein spots were compared (Dyballa and Metzger, 2009). For immunoblotting experiments the gels were transferred to nitrocellulose membrane and immunoblotted using different antibodies.

### In-Gel Digestion and Mass Spectrometry:

In-Gel digestions of the excised gel-spots were performed as follows. The spots were sliced, destained, dehydrated and dried. the dried gel slices were then rehydrated in 20 μl of 12 ng/μl Trypsin Gold (V5280, Promega) in 0.01% ProteaseMAX™ Surfactant (Trypsin enhancer, V2071, Promega):50 mM NH.1HCO3 for 10 mins and then overlaid with 30 μl of 0.01% ProteaseMAX™ Surfectant:50 mM NH₄HCO₃, gently mixed and incubated over night at 37° C. on a horizontal shaker. The eluted peptides were cleaned by Pierce™ C-18 tips (ThermoFisher, 87782) and analyzed by tandem mass spectrometry (MS) analysis using AB SCIEX TripleTOF™ 5600 System (Applied Biosystems/MOS Sciex, Foster City, Calif.) at the Manitoba Centre for Proteomics and Systems Biology, University of Manitoba.

### Live Cell Confocal Imaging

The mouse TF cDNA was amplified and cloned in pHTN-Halo tag plasmid. The pHTN-Halo-TF plasmids were co-transfected in cultured adult primary DRG neurons using rat neuron nucleofection kit (VPG-1003, Amaxa, Lonza Inc., Allendale, N.J., USA) and Amaxa nucleofector-II apparatus (program 0-003) and cultured on poly L-Ornithine (P8638, Sigma) and laminin coated μ-Plate-24 well dishes (Ibidi USA, Inc. Madison, Wis., USA) for 48 hours. The Halo-TF proteins were labeled using 100 nM cell permeable Halo-Tag TMRDirect ligand (G8251, Promega) and incubated for 15 mins at 37° C. in 5% CO2. The cells were washed, imaged in a LSM510 (Zeiss) confocal microscope.

### Two Dimensional BN-PAGE/SDS-PAGE Analysis:

In 2D BN-PAGE/SDS-PAGE, the first dimension native page separates the multiprotein complexes and the 2^{nd} dimension denatured SDS-PAGE separates the interacting protein components in the MPC which appears on a vertical line (Sabbir et al., 2016). The first dimension BN-PAGE and 2^{nd} dimension SDS-PAGE was performed as described previously (Sabbir et al., 2016). Briefly, the cell lysates were prepared in 1× phosphate-buffered Saline (PBS) supplemented with 1× Halt protease and phosphatase inhibitor cocktail (1861281, Thermo Scientific) and 1.5% n-Dodecyl β-D-maltoside (D4641, Sigma) and sonicated. The proteins were then separated in 4-15% gradient blue-native polyacrylamide gel. The gel strips (individual lanes) were carefully excised including the 3.2% stacking gel and immersed in the Laemmli sample buffer containing freshly prepared DTT (54 mg/ml). The gel slices were incubated in sample buffer for 30 mins at 55° C. temperature and then the proteins in the gel slices were separated in 2^{nd} dimension SDS-PAGE and immunoblotted.

### Western Blotting and Chemiluminescence-Detection:

Relative quantification of proteins was done by SDS-PAGE separation of total proteins followed by transfer to nitrocellulose membrane and immunoblotting based detection using HRP-conjugated secondary antibodies. The chemiluminescence was detected and imaged using ChemiDoc™ imaging system and Image Lab software version 5.0 build-18 (BioRad). Table 2 summarizes all the primary antibodies and other reagents used in this study. The cell lysates were prepared in 1×RIPA lysis and extraction buffer (Cat No: 89900, ThermoFisher Scientific) supplemented with 1× Halt protease and phosphatase inhibitor cocktail (Cat No: 78441, ThermoFisher Scientific).

### Immunofluorescence:

The immunofluorescence detection was performed as follows. Cultured DRG neurons were fixed in 2% paraformaldehyde (pH7.5) for 10 mins, washed in 1× phosphate buffered saline (PBS) and permeabilized with 0.5% Triton X-100 in PBS (PBST) and then blocked using 1% BSA in PBST. The permeabilized neurons were then incubated with primary antibodies, detected with florescence conjugate secondary antibodies and imaged in LSM510 (Zeiss) confocal microscope.

### Statistical Analysis

Statistical analysis was performed using Prism version 7.00 (GraphPad Software). The mean of two or more groups were compared using one-way ANOV A followed by multiple comparison tests (Siegel, 1956; Dunn, 1964). The mean of multiple experimental groups were compared with the control group by Dunnett's post hoc multiple comparison test, whereas, the mean between two experimental groups were compared by Sidak's post hoc multiple comparison test (Dunn, 1964). Comparisons between two groups were performed using Student's t test (unpaired). Differences were considered significant with P<0.05.

The scope of the claims should not be limited by the preferred embodiments set forth in the examples but should be given the broadest interpretation consistent with the description as a whole.

### REFERENCES

Abbaspour, N., Hurrell, R., and Kelishadi, R. (2014). Review on iron and its importance for human health. J Res Med Sci 19, 164-174.
Akude, E., Zherebitskaya, E., Chowdhury, S. K., Smith, D. R., Dobrowsky, R. T., and Fernyhough, P. (2011). Diminished superoxide generation is associated with respiratory chain dysfunction and changes in the mitochondrial proteome of sensory neurons from diabetic rats. Diabetes 60, 288-297.
Alessi, D. R., James, S. R., Downes, C. P., Holmes, AB., Gaffney, P. R., Reese, C. B., and Cohen, P. (1997). Characterization of a 3-phosphoinositide-dependent protein kinase which phosphorylates and activates protein kinase B alpha. Curr Biol 7, 261-269.
Altamura, S., and Muckenthaler, M. U. (2009). Iron toxicity in diseases of aging: Alzheimer's disease, Parkinson's disease and atherosclerosis. J Alzheirners Dis 16, 879-895.
Anderson, K. A., Lin, F., Ribar, T. J., Stevens, RD., Muehlbauer, M. J., Newgard, C. B., and Means, A R. (2012). Deletion of CaMKK2 from the liver lowers blood glucose and improves whole-body glucose tolerance in the mouse. Mol Endocrinol 26, 281-291.
Anderson, K. A., Means, R. L., Huang, Q. H., Kemp, B. E., Goldstein, E. G., Selbert, M. A., Edelman, A M., Fremeau, R T., and Means, A R. (1998). Components of a calmodulin-dependent protein kinase cascade. Molecular cloning, functional characterization and cellular localization of Ca2+/calmodulin-dependent protein kinase kinase beta. J Biol Chem 273, 31880-31889.
Anderson, K. A., Ribar, T. J., Lin, F., Noeldner, P. K., Green, M. F., Muehlbauer, M. J., Witters, L. A., Kemp, B. E., and Means, A R. (2008). Hypothalamic CaMKK2 contributes to the regulation of energy balance. Cell Metab 7, 377-388.
Arber, C. E., Li, A., Houlden, H., and Wray, S. (2016). Review: Insights into molecular mechanisms of disease in neurodegeneration with brain iron accumulation: unifying theories. Neuropathol Appl Neurobiol 42, 220-241.
Arosio, P., Ingrassia, R., and Cavadini, P. (2009). Ferritins: a family of molecules for iron storage, antioxidation and more. Biochim Biophys Acta 1790, 589-599.
Astarita, G., Jung, K. M., Berchtold, N.C., Nguyen, V. Q., Gillen, D. L., Head, E., Cotman, C. W., and Piomelli, D. (2010). Deficient liver biosynthesis of docosahexaenoic acid correlates with cognitive impairment in Alzheimer's disease. PLoS One 5, e12538.
Astarita, G., and Piomelli, D. (2011). Towards a whole-body systems [multi-organ] lipidomics in Alzheimer's disease. Prostaglandins Leukot Essent Fatty Acids 85, 197-203.
Ballabh, P., Braun, A., and Nedergaard, M. (2004). The blood-brain barrier: an overview: structure, regulation, and clinical implications. Neurobiol Dis 16, 1-13.
Ballatore, C., Lee, V. M., and Trojanowski, J. Q. (2007). Tau-mediated neurodegeneration in Alzheimer's disease and related disorders. Nat Rev Neurosci 8, 663-672.
Bartzokis, G., Beckson, M., Hance, D. B., Marx, P., Foster, J. A., and Marder, S. R. (1997). MR evaluation of age-related increase of brain iron in young adult and older normal males. Magn Reson Imaging 15, 29-35.
Barupala, D. P., Dzul, S. P., Riggs-Gelasco, P. J., and Stemmler, T. L. (2016). Synthesis, delivery and regulation of eukaryotic heme and Fe—S cluster cofactors. Arch Biochem Biophys 592, 60-75.
Berridge, M. J. (2011). Calcium signalling and Alzheimer's disease. Neurochem Res 36, 1149-1156.
Bogdan, A. R., Miyazawa, M., Hashimoto, K., and Tsuji, Y. (2016). Regulators of Iron Homeostasis: New Players in Metabolism, Cell Death, and Disease. Trends Biochem Sci 41, 274-286.
Bradbury, M. W. (1997). Transport of iron in the blood-brain-cerebrospinal fluid system. J Neurochem 69, 443-454.
Brightman, M. W., Klatzo, I., Olsson, Y., and Reese, T. S. (1970). The blood-brain barrier to proteins under normal and pathological conditions. J Neural Sci 10, 215-239.
Calcutt, N. A., Smith, D. R., Frizzi, K., Sabbir, M. G., Chowdhury, S. K., Mixcoatl-Zecuatl, T., Saleh, A., Muttalib, N., Van Der Ploeg, R., Ochoa, J., Gopaul, A., Tessler, L., Wess, J., Jolivalt, C. G., and Fernyhough, P. (2017). Selective antagonism of muscarinic receptors is neuroprotective in peripheral neuropathy. J Clin Invest 127, 608-622.
Campuzano, V., Montermini, L., Molto, M. D., Pianese, L., Cossee, M., Cavalcanti, F., Monros, E., Rodius, F., Duclos, F., Monticelli, A., Zara, F., Canizares, J., Koutnikova, H., Bidichandani, S. I., Gellera, C., Brice, A., Trouillas, P., De Michele, G., Filla, A., De Frutos, R., Palau, F., Patel, P. I., Di Donato, S., Mandel, J. L., Cocozza, S., Koenig, M., and Pandolfo, M. (1996). Friedreich's ataxia: autosomal recessive disease caused by an intronic GAA triplet repeat expansion. Science 271, 1423-1427.
Cao, W., Sohail, M., Liu, G., Koumbadinga, G. A., Lobo, V. G., and Xie, J. (2011). Differential effects of PKA-controlled CaMKK2 variants on neuronal differentiation. RNA Biol 8, 1061-1072.
Carvajal, J. J., Pook, M. A., Dos Santos, M., Doudney, K., Hillermann, R., Minogue, S., Williamson, R., Hsuan, J. J., and Chamberlain, S. (1996). The Friedreich's ataxia gene encodes a novel phosphatidylinositol-4-phosphate 5-kinase. Nat Genet 14, 157-162.
Cheng, Y., Zak, 0., Aisen, P., Harrison, S. C., and Walz, T. (2004). Structure of the human transferrin receptor-transferrin complex. Cell 116, 565-576.
Cheung, Z. H., and Ip, N.Y. (2012). Cdk5: a multifaceted kinase in neurodegenerative diseases. Trends Cell Biol 22, 169-175.
Chiasserini, D., Van Weering, J. R., Piersma, S. R., Pham, T. V., Malekzadeh, A., Teunissen, C. E., De Wit, H., and Jimenez, C. R. (2014). Proteomic analysis of cerebrospinal fluid extracellular vesicles: a comprehensive dataset. J Proteomics 106, 191-204.
Chou, M. M., Hou, W., Johnson, J., Graham, L. K., Lee, M. H., Chen, C. S., Newton, A. G., Schaffhausen, B. S., and Toker, A. (1998). Regulation of protein kinase C zeta by Pl 3-kinase and PDK-1. Curr Biol 8, 1069-1077.
Connor, J. R., Menzies, S. L., St Martin, S. M., and Mufson, E. J. (1992). A histochemical study of iron, transferrin, and ferritin in Alzheimer's diseased brains. J Neurosci Res 31, 75-83.
Corcoran, E. E., and Means, A. R. (2001). Defining Ca2+/calmodulin-dependent protein kinase cascades in transcriptional regulation. J Biol Chem 276, 2975-2978.
Dagda, R. K., and Das Banerjee, T. (2015). Role of protein kinase A in regulating mitochondrial function and neuronal development: implications to neurodegenerative diseases. Rev Neurosci 26, 359-370.
Dedman, D. J., Treffry, A., Candy, J. M., Taylor, G. A., Morris, C. M., Bloxham, C. A., Perry, R. H., Edwardson, J. A., and Harrison, P. M. (1992). Iron and aluminium in relation to brain ferritin in normal individuals and Alzheimer's-disease and chronic renal-dialysis patients. Biochem J 287 (Pt 2), 509-514.
Dickinson, T. K., and Connor, J. R. (1994). Histological analysis of selected brain regions of hypotransferrinemic mice. Brain Res 635, 169-178.
Dunn, O. J. (1964). Multiple Comparisons Using Rank Sums. Technometrics 6, 241-252.
Dyballa, N., and Metzger, S. (2009). Fast and sensitive colloidal coomassie G-250 staining for proteins in polyacrylamide gels. J Vis Exp.
Esiri, M. M., and Wilcock, G. K. (1984). The olfactory bulbs in Alzheimer's disease. J Neural Neurosurg Psychiatry 4 7, 56-60.
Fillebeen, C., Descamps, L., Dehouck, M. P., Fenart, L., Benaissa, M., Spik, G., Cecchelli, R., and Pierce, A. (1999). Receptor-mediated transcytosis of lactoferrin through the blood-brain barrier. J Biol Chem 274, 7011-7017.
Fleming, R. E., and Ponka, P. (2012). Iron overload in human disease. N Engl J Med 366, 348-359.
Frame, S., and Cohen, P. (2001). GSK3 takes centre stage more than 20 years after its discovery. Biochem J 359, 1-16.
Freeman, W. M., and Hemby, S. E. (2004). Proteomics for protein expression profiling in neuroscience. Neurochem Res 29, 1065-1081.
Garcia-Mesa, Y., Lopez-Ramos, J. C., Gimenez-Llort, L., Revilla, S., Guerra, R., Gruart, A., Laferla, F. M., Cristofol, R., Delgado-Garcia, J. M., and Sanfeliu, C. (2011). Physical exercise protects against Alzheimer's disease in 3xTg-AD mice. J Alzheimers Dis 24, 421-454.
Gardner, B., Dieriks, B. V., Cameron, S., Mendis, L. H. S., Turner, C., Faull, R. L. M., and Curtis, M. A. (2017). Metal concentrations and distributions in the human olfactory bulb in Parkinson's disease. Sci Rep 7, 10454.
Gasteiger, E., Gattiker, A., Hoogland, C., Ivanyi, I., Appel, R. D., and Bairoch, A. (2003). ExPASy: The proteomics server for in-depth protein knowledge and analysis. Nucleic Acids Res 31, 3784-3788.
Gimenez-Llort, L., Garcia, Y., Buccieri, K., Revilla, S., Sunol, C., Cristofol, R., and Sanfeliu, C. (2010). Gender-Specific Neuroimmunoendocrine Response to Treadmill Exercise in 3xTg-AD Mice. Int J Alzheimers Dis 2010, 128354.
Goedert, M. (2001). Alpha-synuclein and neurodegenerative diseases. Nat Rev Neurosci 2, 492-501.
Golts, N., Snyder, H., Frasier, M., Theisler, C., Choi, P., and Wolozin, B. (2002). Magnesium inhibits spontaneous and iron-induced aggregation of alpha-synuclein. J Biol Chem 277, 16116-16123.
Gomme, P. T., McCann, K. B., and Bertolini, J. (2005). Transferrin: structure, function and potential therapeutic actions. Drug Discov Today 10, 267-273.
Good, P. F., Perl, D. P., Bierer, L. M., and Schmeidler, J. (1992). Selective accumulation of aluminum and iron in the neurofibrillary tangles of Alzheimer's disease: a laser microprobe (LAMMA) study. Ann Neural 31, 286-292.
Gordon, N. (2000). Friedreich's ataxia and iron metabolism. Brain Dev 22, 465-468.
Green, M. F., Scott, J. W., Steel, R., Oakhill, J. S., Kemp, B. E., and Means, A R. (2011). Ca2+/Calmodulin-dependent protein kinase kinase beta is regulated by multisite phosphorylation. J Biol Chem 286, 28066-28079.
Gregory, A., and Hayflick, S. (1993). “Neurodegeneration with Brain Iron Accumulation Disorders Overview,” in GeneReviews((R)), eds. M. P. Adam, H. H. Ardinger, RA. Pagon, S. E. Wallace, L. J. H. Bean, H. C. Mefford, K. Stephens, A. Amemiya & N. Ledbetter. (Seattle (Wash.)).
Guevara, J., Espinosa, B., Zenteno, E., Vazguez, L., Luna, J., Perry, G., and Mena, R. (1998). Altered glycosylation pattern of proteins in Alzheimer disease. J Neuropathol Exp Neural 57, 905-914.
Guo, C. C., Tan, R., Hodges, J. R., Hu, X., Sarni, S., and Hornberger, M. (2016). Network-selective vulnerability of the human cerebellum to Alzheimer's disease and frontotemporal dementia. Brain 139, 1527-1538.
Hare, D. J., Doecke, J. D., Faux, N. G., Rembach, A., Volitakis, I., Fowler, C. J., Grimm, R., Doble, P. A., Cherny, RA., Masters, C. L., Bush, A. I., and Roberts, B. R. (2015). Decreased plasma iron in Alzheimer's disease is due to transferrin desaturation. ACS Chem Neurosci 6, 398-402.
Hashimoto, M., Hsu, L. J., Xia, Y., Takeda, A., Sisk, A., Sundsmo, M., and Masliah, E. (1999). Oxidative stress induces amyloid-like aggregate formation of NACP/alpha-synuclein in vitro. Neuroreport 10, 717-721.
Haste Andersen, P., Nielsen, M., and Lund, 0. (2006). Prediction of residues in discontinuous 8-cell epitopes using protein 3D structures. Protein Sci 15, 2558-2567.
Hebda-Bauer, E. K., Simmons, T. A., Sugg, A., Ural, E., Stewart, J. A., Beals, J. L., Wei, Q., Watson, S. J., and Akil, H. (2013). 3xTg-AD mice exhibit an activated central stress axis during early-stage pathology. J Alzheimers Dis 33, 407-422.
Hermes, M., Eichhoff, G., and Garaschuk, 0. (2010). Intracellular calcium signalling in Alzheimer's disease. J Cell Mol Med 14, 30-41.
Herrup, K., and Wilczynski, S. L. (1982). Cerebellar cell degeneration in the leaner mutant mouse. Neuroscience 7, 2185-2196.
Hirata-Fukae, C., Li, H. F., Hoe, H. S., Gray, A. J., Minami, S. S., Hamada, K., Niikura, T., Hua, F., Tsukagoshi-Nagai, H., Horikoshi-Sakuraba, Y., Mughal, M., Rebeck, G. W., Laferla, F. M., Mattson, M. P., Iwata, N., Saido, T. C., Klein, W. L, Duff, K. E., Aisen, P. S., and Matsuoka, Y. (2008). Females exhibit more extensive amyloid, but not tau, pathology in an Alzheimer transgenic model. Brain Res 1216, 92-103.
Hsu, LS., Chen, G. D., Lee, LS., Chi, C. W., Cheng, J. F., and Chen, J. Y. (2001). Human Ca2+/calmodulin-dependent protein kinase kinase beta gene encodes multiple isoforms that display distinct kinase activity. J Biol Chem 276, 31113-31123.
Huynh, R. A., and Mohan, C. (2017). Alzheimer's Disease: Biomarkers in the Genome, Blood, and Cerebrospinal Fluid. Front Neural 8, 102.
Jacobs, H. I. L, Hopkins, D. A., Mayrhofer, H. C., Bruner, E., Van Leeuwen, F. W., Raaijmakers, W., and Schmahmann, J. D. (2018). The cerebellum in Alzheimer's disease: evaluating its role in cognitive decline. Brain 141, 37-47.
Kahl, C. R., and Means, A. R. (2004). Regulation of cyclin D1/Cdk4 complexes by calcium/calmodulin-dependent protein kinase I. J Biol Chem 279, 15411-15419.
Kawamoto, E. M., Vivar, C., and Camandola, S. (2012). Physiology and pathology of calcium signaling in the brain. Front Pharmacol 3, 61.
Kawauchi, T. (2014). Cdk5 regulates multiple cellular events in neural development, function and disease. Dev Growth Differ 56, 335-348.
Ke, Y., and Ming Qian, Z. (2003). Iron misregulation in the brain: a primary cause of neurodegenerative disorders. Lancet Neural 2, 246-253.
Ke, Y., and Qian, Z. M. (2007). Brain iron metabolism: neurobiology and neurochemistry. Prog Neurobiol 83, 149-173.
Kim, D. G., Krenz, A., Toussaint, LE., Maurer, K. J., Robinson, S. A., Yan, A., Torres, L, and Bynoe, M. S. (2016). Non-alcoholic fatty liver disease induces signs of Alzheimer's disease (AD) in wild-type mice and accelerates pathological signs of AD in an AD model. J Neuroinflammation 13, 1.
Kockeritz, L, Doble, B., Patel, S., and Woodgett, J. R. (2006). Glycogen synthase kinase-3—an overview of an over-achieving protein kinase. Curr Drug Targets 7, 1377-1388.
Kokubo, M., Nishio, M., Ribar, T. J., Anderson, K. A., West, A. E., and Means, A. R. (2009). BDNF-mediated cerebellar granule cell development is impaired in mice null for CaMKK2 or CaMKIV. J Neurosci 29, 8901-8913.
Kovacs, T., Cairns, N.J., and Lantos, P. L (2001). Olfactory centres in Alzheimer's disease: olfactory bulb is involved in early Braak's stages. Neuroreport 12, 285-288.
Kringelum, J. V., Lundegaard, C., Lund, 0., and Nielsen, M. (2012). Reliable B cell epitope predictions: impacts of method development and improved benchmarking. PLoS Comput Biol 8, e1002829.
Kringelum, J. V., Nielsen, M., Padkjaer, S. S., and Lund, 0. (2013). Structural analysis of 8-cell epitopes in antibody:protein complexes. Mol Immunol 53, 24-34.
Larsen, J. E., Lund, 0., and Nielsen, M. (2006). Improved method for predicting linear B-cell epitopes. Immunome Res 2, 2.
Lehmann, S., and Teunissen, C. E. (2016). Editorial: Biomarkers of Alzheimer's Disease: The Present and the Future. Front Neural 7, 158.
Levi, S., and Finazzi, D. (2014). Neurodegeneration with brain iron accumulation: update on pathogenic mechanisms. Front Pharmacol 5, 99.
Lin, F., Marcelo, K. L, Rajapakshe, K., Coarfa, C., Dean, A, Wilganowski, N., Robinson, H., Sevick, E., Bissig, K. D., Goldie, L. C., Means, A R., and York, B. (2015). The camKK2/camKIV relay is an essential regulator of hepatic cancer. Hepatology 62, 505-520.
Lin, F., Ribar, T. J., and Means, A R. (2011). The Ca2+/calmodulin-dependent protein kinase kinase, CaMKK2, inhibits preadipocyte differentiation. Endocrinology 152, 3668-3679.
Lista, S., Faltraco, F., Prvulovic, 0., and Hampel, H. (2013). Blood and plasma-based proteomic biomarker research in Alzheimer's disease. Prog Neurobiol 101-102, 1-17.
Liu, R., Guan, J. Q., Zak, O., Aisen, P., and Chance, M. R. (2003). Structural reorganization of the transferrin C-lobe and transferrin receptor upon complex formation: the C-lobe binds to the receptor helical domain. Biochemistry 42, 12447-12454.
Mairet-Coello, G., Courchet, J., Pieraut, S., Courchet, V., Maximov, A, and Polleux, F. (2013). The CAMKK2-AMPK kinase pathway mediates the synaptotoxic effects of Abeta oligomers through Tau phosphorylation. Neuron 78, 94-108.
Manning, B. D., and Cantley, LC. (2007). AKT/PKB signaling: navigating downstream. Cell 129, 1261-1274.
Marcelo, K. L, Means, A R., and York, B. (2016). The Ca(2+)/Calmodulin/CaMKK2 Axis: Nature's Metabolic CaMshaft. Trends Endocrinol Metab 27, 706-718.
Mills, E., Dong, X. P., Wang, F., and Xu, H. (2010). Mechanisms of brain iron transport: insight into neurodegeneration and CNS disorders. Future Med Chem 2, 51-64.
Mu, Y., and Gage, F. H. (2011). Adult hippocampal neurogenesis and its role in
Alzheimer's disease. Mol Neurodegener 6, 85.
Neuberger, M. S., Williams, G. T., and Fox, R. O. (1984). Recombinant antibodies possessing novel effector functions. Nature 312, 604-608.
O'Bryant, S. E., Mielke, M. M., Rissman, R. A, Lista, S., Vanderstichele, H., Zetterberg, H., Lewczuk, P., Posner, H., Hall, J., Johnson, L., Fong, Y. L, Luthman, J., Jeromin, A, Batrla-Utermann, R., Villarreal, A, Britton, G., Snyder, P. J., Henriksen, K., Grammas, P., Gupta, V., Martins, R., Hampel, H., and Biofluid Based Biomarker Professional Interest, A (2017). Blood-based biomarkers in Alzheimer disease: Current state of the science and a novel collaborative paradigm for advancing from discovery to clinic. Alzheimers Dement 13, 45-58.
Obenauer, J. C., Cantley, L C., and Yaffe, M. B. (2003). Scansite 2.0: Proteome-wide prediction of cell signaling interactions using short sequence motifs. Nucleic Acids Res 31, 3635-3641.
Oddo, S., Caccamo, A., Shepherd, J. D., Murphy, M. P., Golde, T. E., Kayed, R., Metherate, R., Mattson, M. P., Akbari, Y., and Laferla, F. M. (2003). Triple-transgenic model of Alzheimer's disease with plaques and tangles: intracellular Abeta and synaptic dysfunction. Neuron 39, 409-421.
Ohmstede, C. A., Jensen, K. F., and Sahyoun, N. E. (1989). Ca2+/calmodulin-dependent protein kinase enriched in cerebellar granule cells. Identification of a novel neuronal calmodulin-dependent protein kinase. J Biol Chem 264, 5866-5875.
Oury, F., Yadav, V. K., Wang, Y., Zhou, B., Liu, X. S., Guo, X. E., Tecott, L. H., Schutz, G., Means, A. R., and Karsenty, G. (2010). GREB mediates brain serotonin regulation of bone mass through its expression in ventromedial hypothalamic neurons. Genes Dev 24, 2330-2342.
Ozlu, N., Monigatti, F., Renard, B. Y., Field, C. M., Steen, H., Mitchison, T. J., and Steen, J. J. (2010). Binding partner switching on microtubules and aurora-B in the mitosis to cytokinesis transition. Mol Cell Proteomics 9, 336-350.
Pelkmans, L., Fava, E., Grabner, H., Hannus, M., Habermann, B., Krausz, E., and Zerial, M. (2005). Genome-wide analysis of human kinases in clathrin- and caveolae/raft-mediated endocytosis. Nature 436, 78-86.
Peters, M., Mizuno, K., Ris, L., Angelo, M., Godaux, E., and Giese, K. P. (2003). Loss of Ca2+/calmodulin kinase kinase beta affects the formation of some, but not all, types of hippocampus-dependent long-term memory. J Neurosci 23, 9752-9760.
Philpott, C. C. (2012). Coming into view: eukaryotic iron chaperones and intracellular iron delivery. J Biol Chem 287, 13518-13523.
Puccio, H., Simon, D., Cossee, M., Criqui-Filipe, P., Tiziano, F., Melki, J., Hindelang, C., Matyas, R., Rustin, P., and Koenig, M. (2001). Mouse models for Friedreich ataxia exhibit cardiomyopathy, sensory nerve defect and Fe—S enzyme deficiency followed by intramitochondrial iron deposits. Nat Genet 27, 181-186.
Racioppi, L., and Means, A. R. (2012). Calcium/calmodulin-dependent protein kinase kinase 2: roles in signaling and pathophysiology. J Biol Chem 287, 31658-31665.
Ribar, T. J., Rodriguiz, R. M., Khiroug, L., Wetsel, W. C., Augustine, G. J., and Means, A. R. (2000). Cerebellar defects in Ca2+/calmodulin kinase IV-deficient mice. J Neurosci 20, RC107.
Richardson, D. R., Lane, D. J., Becker, E. M., Huang, M. L., Whitnall, M., Suryo Rahmanto, Y., Sheffel, A. O., and Ponka, P. (2010). Mitochondrial iron trafficking and the integration of iron metabolism between the mitochondrion and cytosol. Proc Natl Acad Sci USA 107, 10775-10782.
Rouault, T. A. (2013). Iron metabolism in the CNS: implications for neurodegenerative diseases. Nat Rev Neurosci 14, 551-564.
Roy Chowdhury, S. K., Smith, D. R., Saleh, A., Schapansky, J., Marquez, A., Gomes, S., Akude, E., Morrow, D., Calcutt, N. A., and Fernyhough, P. (2012). Impaired adenosine monophosphate-activated protein kinase signalling in dorsal root ganglia neurons is linked to mitochondrial dysfunction and peripheral neuropathy in diabetes. Brain 135, 1751-1766.
Rungta, R. L., Choi, H. B., Lin, P. J., Ko, R. W., Ashby, D., Nair, J., Manoharan, M., Cullis, P. R., and Macvicar, B. A. (2013). Lipid Nanoparticle Delivery of siRNA to Silence Neuronal Gene Expression in the Brain. Mol Ther Nucleic Acids 2, e136.
Sabbir, M. G., Dillon, R., and Mowat, M. R. (2016). Dlc1 interaction with non-muscle myosin heavy chain II-A (Myh9) and Racl activation. Biol Open 5, 452-460.
Sakagami, H., Saito, S., Kitani, T., Okuno, S., Fujisawa, H., and Kondo, H. (1998). Localization of the mRNAs for two isoforms of Ca2+/calmodulin-dependent protein kinase kinases in the adult rat brain. Brain Res Mot Brain Res 54, 311-315.
Saleh, A., Roy Chowdhury, S. K., Smith, D. R., Balakrishnan, S., Tessler, L., Martens, C., Morrow, D., Schartner, E., Frizzi, K. E., Calcutt, N. A., and Fernyhough, P. (2013). Ciliary neurotrophic factor activates NF-kappaB to enhance mitochondrial bioenergetics and prevent neuropathy in sensory neurons of streptozotocin-induced diabetic rodents. Neuropharmacology 65, 65-73.
Sawaya, B. E., and Schaeffer, E. (1995). Transcription of the human transferrin gene in neuronal cells. Nucleic Acids Res 23, 2206-2211.
Sbalzarini, I. F., and Koumoutsakos, P. (2005). Feature point tracking and trajectory analysis for video imaging in cell biology. J Struct Biol 151, 182-195.
Serrano-Pozo, A., Frosch, M. P., Masliah, E., and Hyman, B. T. (2011). Neuropathological alterations in Alzheimer disease. Cold Spring Harb Perspect Med 1, a006189.
Sharma, N., and Singh, AN. (2016). Exploring Biomarkers for Alzheimer's Disease. J Clin Diagn Res 10, KE01-06.
Shirotani, K., Futakawa, S., Nara, K., Hoshi, K., Saito, T., Tohyama, Y., Kitazume, S., Yuasa, T., Miyajima, M., Arai, H., Kuno, A., Narimatsu, H., and Hashimoto, Y. (2011). High Throughput ELISAs to Measure a Unique Glycan on Transferrin in Cerebrospinal Fluid: A Possible Extension toward Alzheimer's Disease Biomarker Development. Int J Alzheimers Dis 2011, 352787.
Siegel, S. (ed.). (1956). Nonparametric statistics for the behavioral sciences. New York: McGraw-Hill.
Soderling, T. R. (1999). The Ca-calmodulin-dependent protein kinase cascade. Trends Biochem Sci 24, 232-236.
Soderling, T. R., and Stull, J. T. (2001). Structure and regulation of calcium/calmodulin-dependent protein kinases. Chem Rev 101, 2341-2352.
Squitti, R., Salustri, C., Siotto, M., Ventriglia, M., Vernieri, F., Lupoi, D., Cassetta, E., and Rossini, P. M. (2010). Ceruloplasmin/Transferrin ratio changes in Alzheimer's disease. Int J Alzheimers Dis 2011, 231595.
Sullivan, J. L. (1981). Iron and the sex difference in heart disease risk. Lancet 1, 1293-1294.
Sutcliffe, J. G., Hedlund, P. B., Thomas, E. A., Bloom, F. E., and Hilbush, B. S. (2011). Peripheral reduction of beta-amyloid is sufficient to reduce brain beta-amyloid: implications for Alzheimer's disease. J Neurosci Res 89, 808-814.
Swulius, M. T., and Waxham, M. N. (2008). Ca(2+)/calmodulin-dependent protein kinases. Cell Mol Life Sci 65, 2637-2657.
Takeda, A., Takatsuka, K., Connor, J. R., and Oku, N. (2001). Abnormal iron accumulation in the brain of neonatal hypotransferrinemic mice. Brain Res 912, 154-161.
Takeda, A., Takatsuka, K., Connor, J. R., and Oku, N. (2002). Abnormal iron delivery to the bone marrow in neonatal hypotransferrinemic mice. Biometals 15, 33-36.
Taniguchi, M., Okayama, Y., Hashimoto, Y., Kitaura, M., Jimbo, D., Wakutani, Y., Wada-Isoe, K., Nakashima, K., Akatsu, H., Furukawa, K., Arai, H., and Urakami, K. (2008). Sugar chains of cerebrospinal fluid transferrin as a new biological marker of Alzheimer's disease. Dement Geriatr Cogn Disord 26, 117-122.
Teh, E. M., Hewitt, J., Ung, K. C., Griffiths, T. A., Nguyen, V., Briggs, S. K., Mason, AB., and Macgillivray, R. T. (2005). Identification of the epitope of a monoclonal antibody that disrupts binding of human transferrin to the human transferrin receptor. FEBS J 272, 6344-6353.
Tellechea, P., Pujol, N., Esteve-Belloch, P., Echeveste, B., Garcia-Eulate, M. R., Arbizu, J., and Riverol, M. (2018). Early- and late-onset Alzheimer disease: Are they the same entity? Neurologia 33, 244-253.
Thornton, C., Bright, N.J., Sastre, M., Muckett, P. J., and Carling, D. (2011). AMP-activated protein kinase (AMPK) is a tau kinase, activated in response to amyloid beta-peptide exposure. Biochem J 434, 503-512.
Trenor, C. C., 3rd, Campagna, D. R., Sellers, V. M., Andrews, N.C., and Fleming, M. D. (2000). The molecular defect in hypotransferrinemic mice. Blood 96, 1113-1118.
Uhlen, M., Fagerberg, L., Hallstrom, B. M., Lindskog, C., Oksvold, P., Mardinoglu, A., Sivertsson, A., Kampf, C., Sjostedt, E., Asplund, A., Olsson, I., Edlund, K., Lundberg, E., Navani, S., Szigyarto, C. A., Odeberg, J., Djureinovic, D., Takanen, J. O., Hober, S., Alm, T., Edqvist, P. H., Berling, H., Tegel, H., Mulder, J., Rockberg, J., Nilsson, P., Schwenk, J. M., Hamsten, M., Von Feilitzen, K., Forsberg, M., Persson, L., Johansson, F., Zwahlen, M., Von Heijne, G., Nielsen, J., and Ponten, F. (2015). Proteomics. Tissue-based map of the human proteome. Science 347, 1260419.
Uhlen, M., Zhang, C., Lee, S., Sjostedt, E., Fagerberg, L., Bidkhori, G., Benfeitas, R., Arif, M., Liu, Z., Edfors, F., Sanli, K., Von Feilitzen, K., Oksvold, P., Lundberg, E., Hober, S., Nilsson, P., Mattsson, J., Schwenk, J. M., Brunnstrom, H., Glimelius, B., Sjoblom, T., Edqvist, P. H., Djureinovic, D., Micke, P., Lindskog, C., Mardinoglu, A., and Ponten, F. (2017). A pathology atlas of the human cancer transcriptome. Science 357.
Van Rensburg, S. J., Berman, P. A., Potocnik, F. C., and Taljaard, J. J. (2000). Glycosylation of transferrin in Alzheimer's disease and alcohol-induced dementia. Metab Brain Dis 15, 243-247.
Vinet, J., Carra, S., Blom, J. M., Harvey, M., Brunello, N., Barden, N., and Tascedda, F. (2003). Cloning of mouse Ca2+/calmodulin-dependent protein kinase kinase beta (CaMKKbeta) and characterization of CaMKKbeta and CaMKKalpha distribution in the adult mouse brain. Brain Res Mol Brain Res 111, 216-221.
Wally, J., Halbrooks, P. J., Vonrhein, C., Rould, M. A., Everse, S. J., Mason, A. B., and Buchanan, S. K. (2006). The crystal structure of iron-free human serum transferrin provides insight into inter-lobe communication and receptor binding. J Biol Chem 281, 24934-24944.
Wayman, G. A., Kaech, S., Grant, W. F., Davare, M., Impey, S., Tokumitsu, H., Nozaki, N., Banker, G., and Soderling, T. R. (2004). Regulation of axonal extension and growth cone motility by calmodulin-dependent protein kinase I. J Neurosci 24, 3786-3794.
Wayman, G. A., Lee, Y. S., Tokumitsu, H., Silva, A. J., Silva, A., and Soderling, T. R. (2008). Calmodulin-kinases: modulators of neuronal development and plasticity. Neuron 59, 914-931.
Wayman, G. A., Tokumitsu, H., and Soderling, T. R. (1997). Inhibitory cross-talk by cAMP kinase on the calmodulin-dependent protein kinase cascade. J Biol Chem 272, 16073-16076.
Xu, G., Liu, R., Zak, 0., Aisen, P., and Chance, M. R. (2005). Structural allostery and binding of the transferrin*receptor complex. Mol Cell Proteomics 4, 1959-1967.
Yang, N., Zhang, H., Wang, M., Hao, Q., and Sun, H. (2012). Iron and bismuth bound human serum transferrin reveals a partially-opened conformation in the N-lobe. Sci Rep 2, 999.
Yu, H. L., Chertkow, H. M., Bergman, H., and Schipper, H. M. (2003). Aberrant profiles of native and oxidized glycoproteins in Alzheimer plasma. Proteomics 3, 2240-2248.
Zhang, L., Luo, S., and Zhang, B. (2016). Glycan analysis of therapeutic glycoproteins. MAbs 8, 205-215.
Zou, Y. M., Lu, D., Liu, LP., Zhang, H. H., and Zhou, Y. Y. (2016). Olfactory dysfunction in Alzheimer's disease. Neuropsychiatr Dis Treat 12, 869-875.

TABLE 1
List of phosphopeptides identified by LC-MS/MS analysis. log(e) for peptides
represent the expectation value for the spectrum-to-sequence assignment.

| | | | |
|---|---|---|---|
| | | | Phospho- |
| | | | amino acid |
| Log(e) | Start | Sequence | residues |

GPM10000002011

| | | | | |
|---|---|---|---|---|
| -2.7 | 251 | RKPVDEYKDCHLAQVPSHTVVAR | Y257 | SEQ ID NO: 1 |
| -4.1 | 333 | YLGYEYVTAIRNLREGTCPEAPTDECKPVK | Y333; Y338 | SEQ ID NO: 2 |
| -4.1 | 333 | YLGYEYVTAIRNLREGTCPEAPTDECKPVK | Y336; Y338 | SEQ ID NO: 3 |
| -5.8 | 391 | ETTEDCIAKIMNGEADAMSLDGGFVYIAGK | T392; T393 | SEQ ID NO: 4 |
| -5.8 | 391 | ETTEDCIAKIMNGEADAMSLDGGFVYIAGK | T392; T393 | SEQ ID NO: 5 |
| -2.4 | 490 | INHCRFDEFFSEGCAPGSK | S500 | SEQ ID NO: 6 |
| -5.2 | 509 | KDSSLCKLCMGSGLNLCEPNNK | S511 | SEQ ID NO: 7 |
| -5.2 | 509 | KDSSLCKLCMGSGLNLCEPNNK | S512 | SEQ ID NO: 8 |
| -2 | 572 | NLNEKDYELLCLDGTRKP | T586 | SEQ ID NO: 9 |

GPM10000002014

| | | | | |
|---|---|---|---|---|
| -3.9 | 391 | ETTEDCIAKIMNGEADAMSLDGGFVYIAGK | S409; T392 | SEQ ID NO: 10 |
| -3.9 | 391 | ETTEDCIAKIMNGEADAMSLDGGFVYIAGK | S409; T393 | SEQ ID NO: 11 |
| -3.2 | 381 | SVGKIECVSEATTEDCIAKIMNGEADAMSLDGGFVYIA | S381; S389 | SEQ ID NO: 12 |
| | | GK | | |
| -3.2 | 381 | SVGKIECVSEATTEDCIAKIMNGEADAMSLDGGFVYIA | S381; T392 | SEQ ID NO: 13 |
| | | GK | | |
| -3.2 | 381 | SVGKIECVSEATTEDCIAKIMNGEADAMSLDGGFVYIA | S381; T393 | SEQ ID NO: 14 |
| | | GK | | |
| -3.2 | 381 | SVGKIECVSEATTEDCIAKIMNGEADAMSLDGGFVYIA | S389; T392 | SEQ ID NO: 15 |
| | | GK | | |
| -3.2 | 381 | SVGKIECVSEATTEDCIAKIMNGEADAMSLDGGFVYIA | S389; T393 | SEQ ID NO: 16 |
| | | GK | | |
| -1.5 | 381 | SVGKIECVSEATTEDCIAKIMNGEADAMSLDGGFVYIA | S381 | SEQ ID NO: 17 |
| | | GK | | |
| -1.4 | 490 | INHCRFDEFFSEGCAPGSK | S500 | SEQ ID NO: 18 |

TABLE 2
Antibodies and reagents used.

| Name | Nature | Source | Cat. No. | Lot No. |
|---|---|---|---|---|
| Mouse | Forward Primer (Sac-II): | Thermofisher | | |
| Transferrin | ATGC CCG CGG | | | |
| cDNA | ATGAGGCTCACCGTGGG | | | |
| amplification | TGCCCTG | | | |
| primers | SEQ ID NO: 19 | | | |
| | Reverse Primer (Not-I): | Thermofisher | | |
| | GCAT G CGGCCGCAA | | | |
| | TTAATGTTTGTGGAAAG | | | |
| | TGCAGGCTTCCAGG | | | |
| | SEQ ID NO: 20 | | | |
| pHTN Halo Tag ® | Halo-tag cloning | Promega | G7721 | |
| CMV-neo | plasmid | | | |
| Vector | | | | |
| Halo-TMR ligand | Labeling Halo-tag | Promega | G825A | 163983 |
| | Recombinant protein | | | |

Antibodies

| Name | Source | Type | Host Sp. | Cat. No | Lot No. |
|---|---|---|---|---|---|
| CaMKK2(ZZ9) | SCBT | Monoclonal | Mouse | Sc-100364 | I2914 |
| B-23(NA24) | SCBT | Monoclonal | Mouse | Sc53175 | F2212 |
| VDAC1(B-6) | SCBT | Monoclonal | Mouse | Sc-390996 | C0116 |
| OXPHOS | Abcam | Monoclonal | Mouse | MS601 | H3829 |
| α-tubulin (TU-02) | SCBT | Monoclonal | Mouse | Sc-8035 | C2017 |
| ERK1/2(C-9) | SCBT | Monoclonal | Mouse | Sc-514302 | G3115 |
| ERK1(C-16) | SCBT | Polyclonal | Rabbit | Sc-93 | B0916 |
| Rab11(A-6) | SCBT | Monoclonal | Mouse | Sc-66912 | E2615 |
| RabS(D-11) | SCBT | Monoclonal | Mouse | Sc-46692 | H1716 |
| Histone-1(G-1) | SCBT | Monoclonal | Mouse | Sc-395530 | G1514 |
| GAPDH(0411) | SCBT | Monoclonal | Mouse | Sc-47724 | 1015 |

1.-33. (canceled)

34. A method for determining the risk of developing a cognitive disorder in a subject, comprising:
a) obtaining a biological sample from a subject at risk for developing Alzheimer's disease;
b) analyzing the biological sample to determine the level of phosphorylation of amino acid residues of interest in a transferrin protein; and
c) comparing the level of phosphorylation obtained to a reference value, wherein an increase in the level of phosphorylation of the transferrin protein compared to the reference value is indicative that said subject has a high risk of developing a cognitive disorder.

35. The method of claim 34, wherein the cognitive disorder is Alzheimer's disease.

36. The method of claim 35, wherein the therapy is effective against one or more of the occurrence, symptoms, or duration of the Alzheimer's disease.

37. The method of claim 34, wherein the subject is a human subject.

38. The method of claim 34, wherein the increase in the level of phosphorylation of the transferrin protein compared to the reference value is indicative that the subject is a candidate to receive a therapy for the prevention and/or treatment of the cognitive disorder.

39. The method of claim 34, wherein the reference value corresponds to a level of phosphorylated transferrin protein from a healthy individual.

40. The method of claim 34, wherein the biological sample is selected from the group consisting of: cerebrospinal fluid, blood serum, blood plasma, blood, and peripheral blood mononuclear cells.

41. The method of claim 34, wherein the phosphorylation of the transferrin protein is indicative that the subject has Alzheimer's disease.

42. The method of claim 34, wherein the transferrin protein is human transferrin protein, and wherein the transferrin is found to be phosphorylated at one or more serine, tyrosine and/or threonine residues selected from the group consisting of: K359; K37; K508; K546; S136; S144; S298; S305; S306; S381, S389, S409, S468; S500, S511, S512, S520; S63; S688; T139; T340; T349; T355; T36; T392, T393, T476; T537; T586, T654; T686; T694;
Y155; Y207; Y257; Y333, Y338, Y336, Y533; Y534; Y536; Y593; Y64; Y666; Y669; and
Y674, and combinations thereof.

43. The method of claim 34, wherein analyzing the biological sample comprises determining the fraction of the transferrin protein having a pH of between 3 and 4.

44. The method of claim 43, wherein the method of analyzing the biological sample comprises performing an immunoblot of the biological sample.

45. A method for screening an individual who is at risk of dementia for a dementia diagnosis comprising:
providing a biological sample from the individual;
determining a profile of phosphorylation levels of transferring proteins in the biological sample; and
comparing the profile of the sample to a reference value, wherein for a positive result, the profile of phosphorylation of transferrin proteins in the sample and the reference value are different.

46. The method of claim 45, wherein the transferrin profile is determined by a level of at least one isoelectric point fraction of transferrin in the sample.

47. The method of claim 45, wherein the transferrin protein is human transferrin protein, and wherein the transferrin is found to be phosphorylated at one or more serine, tyrosine and/or threonine residues selected from the group consisting of: K359; K37; K508; K546; S136; S144; S298; S305; S306; S381, S389, S409, S468; S500, S511, S512, S520; S63; S688; T139; T340; T349; T355; T36; T392, T393, T476; T537; T586, T654; T686; T694; Y155; Y207; Y257; Y333, Y338, Y336, Y533; Y534; Y536; Y593; Y64; Y666; Y669; and Y674, and combinations thereof.
